(19) United States
(12) Patent Application Publication
(10) Pub. No.: US 2002/0106375 A1
(43) Pub. Date: Aug. 8, 2002

(54) Non-cytolytic soluble factor from activated-expanded CD4 cells

(76) Inventors: Pierre L. Triozzi, Birmingham, AL (US); John L. Ridihalgh, Columbus, OH (US); Herbert S. Bresler, Bexley, OH (US)

Correspondence Address: MUELLER AND SMITH, LPA, MUELLER-SMITH BUILDING, 7700 RIVERS EDGE DRIVE, COLUMBUS, OH, 43235

(21) Appl. No.: 09/727,198

(22) Filed: Nov. 30, 2000

Related U.S. Application Data
(60) Provisional application No. 60/246,802, filed on Nov. 8, 2000.

(51) Int. Cl. A61K 38/00; A61K 39/42

(52) U.S. Cl. 424/148.1; 514/12; 424/93.7

(57) ABSTRACT

A new factor, Factor C, is produced by the activated-expanded autologous cells of cancer patients, HIV-1 infected patients, CFS patients, healthy patients, etc. Factor C has a molecular weight of about 70,000 to 80,000 daltons, is heat stable, has an amino acid sequence that is absent from the National Center for Biotechnology Information database, and whose amino acid sequence is not homologous to TNF family ligands. Factor C is derived from CD4 cells in a much greater quantity than from CD8 cells, and is derived from lymph cells in a greater quantity than from PBL cells. A double activation and expansion (activation-expansion) process using immobilized and soluble anti-CD3 mAb makes such Factor C. Factor C appears to inhibit transcription in virally-infected and tumor cells, and stimulates the proliferation of normal lymphocytes. Factor C exhibits synergistic activity with topoisomerase I, topoisomerase II, microtubule, and thymidylate synthetase active agents; is responsible for the synergistic induction of apoptosis; its effect is not secondary to enhanced cell cycling; inhibits the anti-apoptotic factor, NFKB implicated in chemoresistance; enhances uptake of doxorubicin in multi-drug resistant cells, increases covalent topoisomerase I-DNA complexes with topoisomerase I active drugs; and decreases thymidylate synthetase transcription in combination with 5-flurouracil. Factor C with the hormonal agent, tamoxifen, is responsible for the synergistic induction of apoptosis and exhibits synergism in estrogen-receptor-negative cell lines.

### CROSS-REFERENCE TO RELATED APPLICATIONS

This application is based on provisional application serial No. 60/246802, filed on Nov. 9, 2000, the disclosure of which is expressly incorporated herein by reference. This application also is cross-referenced to application Ser. No. 09/943,993, filed on Oct. 3, 1997, and application Ser. No. 09/167,764, filed Oct. 7, 1998, the disclosures of which are expressly incorporated herein by reference.

### STATEMENT REGARDING FEDERALLY SPONSORED RESEARCH

Not applicable.

### BACKGROUND OF THE INVENTION

The present invention relates to a newly-discovered factor that has inhibitory effects on HIV-1 and other viruses, as well as on cancer. Moreover, the factor can enhance the activity of chemotherapeutics used to treat cancer patients. Such new factor will be referred to often herein as “Factor C” or “purified factor” or the like.

Activated lymphocytes often are identified infiltrating tumors, and a number of approaches have successfully elicited activated lymphocytes in patients with cancers. The interactions between activated lymphocytes and tumor cells are complex, and the factors that determine whether or not tumor death or tumor escape will result are poorly understood. Several mechanisms are involved in the antitumor activity of activated lymphocytes. Cytolytic T lymphocytes (CTL) and natural killer (NK) cells can mediate cytolysis by granule exocytosis and the release perforin and granzyme after lymphocyte-tumor cell engagement. In addition, apoptosis of tumor targets can be induced through engagement of membrane-bound Fas ligand (mFasL) on the CTL and NK cell with the Fas receptor (FasR) on tumor cells. Several mechanisms also have been proposed to account for the capacity of tumors to evade these killing mechanisms. Ferrone, et al., “Loss of HLA class I antigens by melanoma cells: molecular mechanisms, functional significance and clinical relevance”, Immunol Today, 16:487, 1995; and Tagliaferri, et al., “Tumour cell resistance to non-MHC-restricted lymphocytes: molecular mechanisms and clinical implications”, Cancel Immunol Immunother, 46:121-7, 1998. Modulation of FasL and/or FasR, which are members of the tumor necrosis factor (TNF) family of ligands and receptors, may be involved. After malignant transformation, tumor FasR may be lost or rendered non-functional. In addition, tumors may express FasL and, thus, not present a “counter-attack” and induce apoptosis of FasR-expressing activated lymphocytes. O° Connell, et al., “The Fas counterattack: Fas-mediated T cell killing by colon cancer cell expression Fas ligand”, J Exp Med, 184:1075, 1996; Tanaka, et al., “Fas ligand in human serum”, Nat Med, 2:317-322, 1996; and Shiraki, et al., “Expression of Fas ligand in liver metastases of human colonic adenocarcinomas”, Proc Natl Acad Sci USA, 94:6420-6425,1997.

Activated lymphocytes release several soluble factors after interacting with tumor cells, which factors may modulate FasL-FasR interactions. Dennert, “Molecular mechanism of target lysis by cytotoxic R cells”, Reviews of Immunology, 14:133-152, 1997. These soluble factors can include cytokines that can inhibit the growth of humor cells and upregulate FasR, such as interferon-γ (IFN-γ). These factors also can include cytokines that can promote cell growth. CTL and NK cells can produce granulocyte macrophage colony stimulating factor (GM-CSF) and transforming growth factor-β (TFG-β), which have been shown to promote cell growth of several non-hematological tumor cell. These cytokines have been shown to downregulate FasR expression. Berdel, et al., “Effects of hematopoietic growth factors on malignant nonhematopoietic cells”, Seminars in Oncology, 19 (Suppl. 4):41-45, 1992); Uhm, et al., “Modulation of transforming growth factor-b1 effects by cytokines”, Immunological Investigations, 22:375-388, 1993); and Spinozzi et al., “Role of T-helper type 2 cytokines in down-modulation of Fas mRNA and receptor on the surface of activated CD4+ T cells: molecular basis for the persistence of the allergic immune response”, FASEB J, 12:1747-1753,1998.

Soluble forms of FasL and other members of the TNF family can be detected in the culture medium of activated lymphocytes, indicating that the TNF family members can be cleaved off from the membrane. Tanaka, et al., “Expression of the functional soluble form of human Fas ligand in activated lymphocytes”, EMBO J. 14:1129-1135,1995; Perez, et al., “A nonsecretable cell surface mutant of tumor necrosis factor (TNF) kills by cell-to-cell contact”, Cell, 63:251-258, 1990; Pietravelle, et al., “Human native soluble CD40L is a biologically active trimer processed inside microsomes”, J Biol Chem, 271:5965-5967, 1996; and Dhein, et al., “Autocrine T cell suicide mediated by APO-1/(Fas/CD95), Nature, 373:438-441, 1995. It recently has been demonstrated that the soluble form of Fas ligand (sFasL), which can be released by activated T cells, not only induces apoptosis less potently than insoluble mFasL, but can antagonize the apoptosis-inducing activity of mFasL and downregulate FasR. Tanaka, et al, “Downregulation of Fas ligand by shedding”, Nature Med, 4:31-36, 1998. Finally, soluble factors released by activated lymphocytes, such as IFN-γ, could upregulate FasL expression by the tumor and potentially its ability to induce apoptosis of the activated lymphocyte.

That immune cells and their cytokines can potentiate the cytotoxicity of cancer chemotherapeutic drugs is well established. Kreuser, et al, “Biochemical modulation of cytotoxic drugs by cytokines: molecular mechanisms in experimental oncology”, Recent Results in Cancer Research, 139:371-382, 1995. A variety of mechanisms have been implicated. Most chemotherapeutics and immune effectors kill tumor cells by a common pathway, i.e., apoptosis. Among the numerous factors known to mediate chemotherapy-related apoptosis, p53 has been the most extensively characterized mechanistically. The p53 gene commonly is altered in human cancer by both mutational and deletional events, and these alterations have been implicated in the failure of tumors to respond to cytotoxic agents. Fisher, “Apoptosis in cancer therapy: crossing the threshold”, Cell, 78a;539-542, 1994). Immune effectors elicit apoptosis by several mechanisms. Prominent among these is the interaction of FasL expressed by NK and cytolytic T lymphocytes with the FasR (CD95) on tumor cells. Fas-mediated apoptosis appears to be p53 independent. Dennert, “Molecular mechanism of target lysis by cytotoxic T cells”, International Reviews of Immunology, 14:133-152, 1997. Immune cells can release a number of cytokines, such as IFN-γ, after tumor engagement. These can effect tumor apoptosis through several pathways, including the Fas system. May, “Control of apoptosis by cytokines”, Adv Pharmacol, 41:219-246, 1997. Chemotherapy-induced apoptosis does not appear to be dependent on the FasL-FasR interaction. Eishcen, et al, “Comparison of apoptosis in wild-type and Fas-resistant cells: chemotherapy-induced apoptosis is not dependent on Fas/Fas ligand interactions”. Blood, 90:935-943, 1997. Studies now have shown, however, that cytotoxic drugs can sensitize cancer cells to Fas-mediated apoptosis effected by activated lymphocytes and vice versa. Micheau, et al., “Sensitization of cancer cells treated with cytotoxic drugs to Fas-mediated cytotoxicity”, J Natl Cancer Inst, 89:783-789, 1997.

The topoisomerase-I-reactive camptothecins, irinotecan, and topotecan, have emerged as important cancer therapeutic agents. Topoisomerase I covalently binds to DNA and causes a single strand break, which results in the relaxation of the supercoiled DNA necessary to replication. Camptothecins interact with the covalent topoisomerase-I-DNA complex preventing the re-ligation of the cleaved DNA—this DNA damage leads to apoptosis. Froelich-Ammon, et al., “Topoisomerase poisons: Harnessing the dark side of enzyme mechanism”, J. Bio. Chem., 270:21429-21432. The combined effects of topoisomerase-I-reactive agents and immunotherapeutics have not been extensively evaluated. IFN-α has been shown to enhance activity of irinotecan, probably through the accumulation of the tumor cells in the S phase. Kobayashi, et al., “Interferon-alpha potentiates the antiproliferative activity of CPT-11 against humor colon cancer xenografts in nude mice”, Anticancer Research, 16:2677-80, 1996. IL-1α has been shown to potentiate the cytotoxicity of camptothecin. IL-1α can increase topoisomerase I-catalyzed camptothecin-induced complexes in vitro. Wang, et al., “Interleukin-1 alpha-induced modulation of topoisomerase I activity and DNA damage: implications in the mechanisms of synergy with camptothecins in vitro and in vivo:, Molecular Pharmacology, 49:269-75, 1996; and Wang, et al., “Potentiation of antitumor activities of carboplatin and camptothecin by interleukin-1 alpha against human ovarian carcinoma in vivo”, Anticancer Research, 14 (5A):1723-6, 1994. Topoisomerases have been implicated in TNF-mediated cytotoxicity, and TNF has been shown to augment the activity of irinotecan. Baloch, et al., “Synergistic interactions between tumor necrosis factor and inhibitors of DNA topoisomerase I and II”, J Immunol, 145:2908-13, 1009; Mori, et al., “Augmentation of antiproliferative activity of CPT-11, a new derivative of camptothecin, by tumor necrosis factor against proliferation of gynecologic tumor cell lines”, Anti-Cancer Drugs, 2:469-74, 1991; and Utsugi, et al., “Potentiation of topoisomerase inhibitor-induced DNA strand breakage and cytotoxicity by tumor necrosis factor: enhancement of topoisomerase activity as a mechanism of potentiation:, Cancer Res, 50:2636-40, 1990.

In the oncology field, U.S. Pat. No. 5,814,295 teaches that excised human lymphocyte cells mitogenically stimulated in the presence of IL-2 and anti-CD3 monoclonal antibody (mAb) can be useful in treating human tumors in vivo. U.S. Pat. No. 6,093,381 teaches that lymph node lymphocytes that have been cultured under mitogenic stimulation conditions or a supernatant of such mitogenically stimulated cultured lymph node lymphocytes can enhance the activity of cancer chemotherapeutic agents. In particular, activity was shown with 5-FU, doxorubicin HCl, etoposide phosphate, irinotecan, and gemcitabine HCl.

In the viral disease field, human immunodeficiency-1 (HIV-1) infection might be controlled by a cellular immune response that is not dependent on classical cytolysis of infected cells, but rather the release of one or more soluble suppressive factors. Levy, et al. have described a factor exclusively produced by CD8+ T cells, the CD8+ antiviral factor (CAF), which blocks viral RNA transcription in an MHC unrestricted fashion. Walker, et al., “CD8+ lymphocytes can control HIV infection in vitro by suppressing virus replication”, Science, 234:1563-1566, 1986; and Mackewicz, et al., “CD8+ cell anti-HIV activity: nonlytic suppression of virus replication”, AIDS Res Hum Retrovirus, 8:629-64, 1992. Lusso, Gallo, et al. have suggested that the chemokines, RANTES, macrophage inflammatory protein 1-α (MIP 1α) and MIP 1β, are the major suppressive factors produced by CD8+ cells and prevent HIV entry into the cell. Cocchi, et al., “Identification of RANTES, MIP 1α and MIP 1β as the major HIV-suppressive factors produced by CD8+ cells”, Science, 270:1811-1815, 1995. Other cytokines, such as IFN-α, IFN-β, TNF-α, transforming growth factor-β (TGF-β), IL-8, and IL-16 also have been implicated as suppressors of HIV-1 replication. Mackewicz, et al., “CD8+ cell anti-HIV activity: nonlytic suppression of virus replication”, AIDS Res Hum Retroviruses, 8:1039-1050, 1992; and Baier, et al., “HIV suppression by interleukin-16: Nature, 378:563, 1995. The interrelationship and relative roles of CAF, the chemokines, and other cytokines in the control of HIV-1 are controversial. The production of CAF has been shown to correlate inversely with disease progression; its structure, however, has not been formally identified. The chemokines have been better structurally characterized; however, HIV-1 infected and noninfected individuals produce comparable amounts, and no correlation has yet been observed between different patterns of disease progression and chemokine concentration. McKenzie, et al., “Serum chemokine levels in patients with non-progressing HIV infection”, AIDS, 10:f29-33, 1996. The relevance of other cytokines, such as TNF-α and IL-16, has been questioned, as their effects are variable and as very high concentrations appear necessary for antiviral activity. Mackewicz, et al., “CD8+ cell anti-HIV activity: nonlytic suppression of virus replication”, AIDS Res Hum Retroviruses, supra; and Clerici, et al., “Soluble HIV suppressive factors: more likely than one Holy Grail””, Immunology Today, 17:297-298,1996.

In application Ser. No. 09/943,993, a method of activating and expanding lymph node lymphocytes ex vivo to maximize the specific secretion of HIV-1 suppressive factors, including CAF and β chemokines is disclosed. See also, Triozzi, et al., “HIV-1-reactive chemokine-producing CD8+ and CD4+ cells expanded from infected lymph nodes”, AIDS Res Hum Retrovirus, in press. A pilot study examined the effects of infusing these cells in HIV-1 infected patients. Triozzi, et al., “Cellular immunotherapy of advanced human immunodeficiency virus-1 infection using autologous, lymph-node lymphocytes: effects on chemokine production”, J Infect Dis, in press. Ten patients, who were maintained on antiretroviral therapy, received a single infusion of the activated-expanded cells. The cell infusion was well tolerated and there were no serious acute or chronic adverse effects, infectious or otherwise. Increase in production of β chemokines by peripheral blood lymphocytes (PBLs) in response to autologous B-cell targets expressing HIV-1 env and increases in serum β chemokine levels were observed. Increases in CD4 and CD8 counts, increases in skin test reactivity to common microbial recall antigens, and decreases in HIV-1 viral load also were observed. The results of this study suggest that these activated-expanded cells may have immunorestorative and antiviral activities in HIV-1-infected patients.

CD8 cells are the source of CAF and the major source of chemokines. A substantial proportion of the activated-expanded cells generated and infused in the pilot clinical trial were CD4. HIV-1 specific CD4+ T-cell responses recently have been reported to be associated with control of viremia. Rosenberg, et al., “Vigorous HIV-1-specific CD4+ T cell responses associated with control of viremia”, Science, 278:1447-1450,1997.

As a final piece to this puzzle, application Ser. No. 09/167,764, filed Oct. 7, 1998, reports a therapeutic agent for treating patients afflicted with chronic fatigue syndrome (CFS), which includes in a pharmaceutically-acceptable carrier, cytokine-producing cells having been produced by the step of subjecting cytokine-producing cells derived from lymph nodes excised from patients afflicted with CFS to mitogenic stimulation in serum-free media for their expansion. Such mitogenic stimulation includes the presence of Interleukin-2 (IL-2) and anti-CD3 monoclonal antibody. In a pilot clinical trial, patients with disease terms as long as 10 years were reported to have responded remarkably to the inventive cellular immunotherapy. The principal investigator reports that a total of 9 patients were subjected to the treatment protocol with 7 responding patients still responding 6-months post treatment. Six of the responders continue to show persistent clinical improvement more than 18 months post-treatment, and no patient that entered the trial is clinically worse than their baseline health status.

Now, it will be observed that the developments reported in treating HIV-1 and other virally-infected patients, cancer patients, and CFS patients with respect to cellular therapy, may have little in common in that the mechanism of action of the treatment reported to not be understood. While such cellular therapies may be efficacious because they spur a cascade of known chemokines, factors, and cytokines that may be responsible for patient improvement and disease suppression, it also may be effective by spawning a yet unreported and unknown Factor C that is responsible for such activity. And while such mechanism of action may seem to be diverse between these diverse diseases, such may not be the case.

### BRIEF SUMMARY OF THE INVENTION

A new factor, Factor C, is produced by the activated-expanded autologous cells of cancer patients, HIV-1 infected patients, CFS patients, healthy patients, etc. Factor C has a molecular weight of about 80,000 daltons, is heat stable, has an amino acid sequence that is absent from the National Center for Biotechnology Information database, and whose amino acid sequence is not homologous to TNF family ligands. Factor C is derived from CD4 cells in a much greater quantity than from CD8 cells, and is derived from lymph cells in a greater quantity than from PBL cells. A double activation and expansion (activation-expansion) process using immobilized and soluble anti-CD3 mAb makes such Factor C. Factor C appears to inhibit transcription in virally-infected and tumor cells, and stimulates the proliferation of normal lymphocytes. Factor C exhibits synergistic activity with topoisomerase I, topoisomerase II, microtubule, and thymidylate synthetase active agents; is responsible for the synergistic induction of apoptosis; its effect is not secondary to enhanced cell cycling; inhibits the anti-apoptotic factor, NFKB implicated in chemoresistance; enhances uptake of doxorubicin in multi-drug resistant cells, increases covalent topoisomerase I-DNA complexes with topoisomerase I active drugs; and decreases thymidylate synthetase transcription in combination with 5-flurouracil. Factor C with the hormonal agent, tamoxifen, is responsible for the synergistic induction of apoptosis and exhibits synergism in estrogen-receptor-negative and estrogen receptor-positive cell lines.

Factor C may be more than one molecule. It may be thought of as a “cytokine” since it is produced by lymphocyte cells or as a “lymphokine”. Regardless, Factor C has been demonstrated to have anti-viral activity as well as anti-tumor activity. Factor C is produced by the activation-expansion of CD4 lymphocyte cells in the presence of anti-CD3 mAb and IL2. The resulting supernatant is subject to fractionation to recover the fraction having a molecular weight of above about 50,000 (50 k) daltons. Such “high” molecular weight supernatant fraction has been demonstrated to exhibit anti-viral activity as well as anti-tumor activity. Factor C is contained in such high molecular weight supernatant fraction. Factor C may be a multimer, i.e., the active form of said protein may be a monomer, dimer, trimer, or even a tetramer; although, dimer or trimer may be more likely.

Factor C itself is a component of the high molecular weight supernatant fraction, which has a molecular weight of about 70,000-80,000 daltons. As noted above, this band may be composed of more than one component. Regardless of its precise composition, such band, or Factor C, has been demonstrated to exhibit anti-viral activity against HIV, herpes simplex virus, and Coxsackie virus; and anti-tumor activity against adenocarcinoma cancers. Based on the work reported in the cross-referenced applications and its efficacy and non-toxicity to lymphocyte cells in vitro, it is believed that Factor C has applicability in the treatment of immune mediated diseases (of which HIV is an example) in both animals and humans. While some of these diseases are bacterial (e.g., tuberculosis) and some are of unknown cause (autoimmune diseases, e.g., rheumatoid arthritis), most such immune mediated diseases are viral induced and result from persistent and acute infections, including latent infection (e.g., human herpes virus), chronic infections (e.g., “old dog encephalitis” following canine distemper virus (CDV) infection or lymphocyteic choriomeningitis in mice), and slow infections (both lentiviruses including HIV, feline immunodeficiency virus (FIV), and simian immunodeficiency virus (SIV); and a group of unclassified agents which cause subacute spongioform encephalopathies including Cruetzfeld-Jakob disease, Kuru, and Mad Cow Disease). Such immunosuppressive or chronic diseases that lead to an immunosuppressed state in the host (both human and animal) should be treatable in accordance with the precepts of the present invention including, for example, HIV, tuberculosis, measles, dengue fever, malaria, hepatitis (chronic), leprosy, rheumatoid arthritis, multiple sclerosis, canine distemper virus, and the like.

Chronic and acute viruses are classified as being DNA viruses or RNA viruses, enveloped and non-enveloped. RNA viruses are exemplified by, for example, picornaviruses, togaviruses, paramyxoviruses, orthomyxoviruses, rhandoviruses, reoviruses, retroviruses, bunyaviruses, coronaviruses, and arenaviruses. DNA viruses are typified by panoviruses, papoviruses, adenoviruses, herpesviruses, and poxviruses. For more information on viruses, reference is made to the following texts: Fenner, et al., Veterinarian Virology, 2nd Edition, Academic Press, New York, New York (1993); Mims, et al., Viral Pathogenesis and Immunology”, Blackwell Scientific Publications, London, England (1984); Virology, B. N. Field, Editor, Raven Press, 3rd edition; Shulman, et al., The Biological and Clinical Basis of Infectious Diseases, 5th edition, W.B. Saunders Co. (1997), the disclosures of which are expressly incorporated herein by reference.

### BRIEF DESCRIPTION OF THE DRAWINGS

For a fuller understanding of the nature and advantages of the present invention, reference should be had to the following detailed description taken in connection with the accompanying drawings, in which:

FIG. 1 is displays the effect of CD4 and CD8 supernatants derived from the lymph nodes and peripheral blood of an HIV-1 infected subject and from the peripheral blood of a normal (HIV-free) volunteer on HIV-1 mRNA expression of lymphocytes cultured from an HIV-1 infected lymph ode in 10 U/ml IL-2, where supernatants were added at 40% vol/vol and HIV-1 mRNA was assessed at 96 hours;

FIG. 2 graphically displays the data recorded in Table 1 on the effects of unfractionated CD4 and CD8 culture supernatants and greater and less than 50 kDa fractions, wherein HIV infection rates were assessed on day 12 by quantitative ELISA for HIV-1 p24 antigen, the data representing the percent suppression on HIV-1 p24 antigen from three different activation-expansions from HIV-1 infected lymph nodes;

FIG. 3 graphically displays the data recorded in Table 2 on the effect of purified fraction, Factor C, on growth of IL-2 cultured CD4 cells without additions (NT), in the presence of HIV-1 (HIV), and with Factor C added at 1:160, 1:40, and 1:10 dilutions, wherein the data displayed represents the fold-increase, namely cell number at day 3, day 6, and day 8, divided by the cell number at culture initiation;

FIG. 4 graphically displays the data recorded in Table 3 on the effect of purified fraction, Factor C, on the HIVj-1 protein expression as determined by flow cytometry with KC57 antibody of IL-2-cultured CD4 cells without additions (NT), in the presence of HIV-1 and with the purified fraction added at 1:160, 1:40, and 1:10 dilutions, the data representing the percent cells positive;

FIG. 5A graphically displays the data recorded in Table 4A on the effect of unfractionated CD4 derived supernatant and a greater and less than 50 kDa fractions on LTR-driven HIV replication as assessed using HeLA-CD4-LTR-β-gal cells, wherein the data represents absorbence mediated by the integrated β-galactasidase gene marker;

FIG. 5B graphically displays the data recorded in Table 4B on the effect of the purified fraction, Factor C, added at 1:160, 1:40, and 1:10 dilutions on LTR driven HIV replication as assessed using HeLA-CD4-LTR-β-gal cells;

FIG. 6 graphically displays the data recorded in Table 5 on the effect of the purified fraction, Factor C, added at 1:30 dilution on LTR-driven HIV replication as assessed using HeLA-CD4-LTR-β-gal cells in the presence and absence of anti-FasL and anti-TNF blocking antibody added at 10 μg/ml, wherein OKT3 was added at 10 μg/ml as a control;

FIG. 7 displays the effect of unfractionated CD4 supernatants and a greater and less than 50 kDa fraction on TNF, TNF receptor, FasL, and NFKβ mRNA of CD4 cells in the presence and absence of HIV;

FIG. 8 shows the mRNA expression of the activated-expanded CD4 cells as determined by flow cytometry for GAPDH (lane 2), FasL (land 3), TRAIL (lane 4) and TNF-α (lane 5), wherein lane 1 is a 100 bp reference;

FIG. 9 shows cytokine and FasL levels, as determined by ELISA, of unstimulated activated-expanded cells and activated-expanded cells stimulated with anti-CD3 mAb or tumor, wherein the data represents the mean±SD for supernatants from 3 different activation-expansions;

FIG. 10 is an immunoblot of FasL produced by activated-expanded T cells (left) and by SW480 cells (right); wherein for T cells, lane 1 represents FasL from supernatants derived from a T cell activation-expansion without further stimulation, lane 2 after stimulation with anti-CD3 mAb, lane 3 represents supernatants derived from a T cell activation-expansion after the T cells had been lysed; wherein for SW480 cells, lane 1 represents the FasL of lysed SW480 supernatants in the absence of stimulation with supernatants, lane 2 in the presence of unfractionated supernatant, and lane 3 in the presence of the supernatants of M_{Γ} greater than 50;

FIG. 11 graphically displays the data recorded in Table 6 on the antiproliferative activity of unstimulated and stimulated supernatants collected at various time points in the activation-expansion and of the media supplemented with 600 MU/ml IL-2 (Media) added at 25% volume/volume to LS174T cells in culture, wherein the data represents the mean±SD for 3 different activation-expansions;

FIG. 12 graphically displays the data recorded in Table 7 on the effects of stimulated supernatant on the growth of colorectal cancer cells added at a range of concentrations;

FIG. 13 graphically displays the data recorded in Table 8 on the effects of stimulated supernatant on the expression of FasR of colorectal cancer cells added at 2.5% or 15% (vol/vol), wherein FasR expression was determined by flow cytometry;

FIG. 14 shows the induction of DNA fragmentation in SW480 cells cultured with stimulated supernatant at 25% (vol/vol), wherein lane 1=no additions, lane 2=media+IL-2 (600 IU/ml), lane 3=unstimulated supernatant, and lane 4=stimulated supernatant;

FIGS. 15A-15C graphically display the data recorded in Tables 9A-9C on the antiproliferative effects of supernatants derived from autologous tumor, unseparated activated-expanded T cell populations derived from lymph nodes (LNL), and CD4 and CD8 cells separated from this population after activation-expansion, wherein supernatants were collected from LNL, CD4, and CD8 populations after stimulation with anti-CD3 mAb (CD3) or with autologous tumor (Tumor), three different activation-expansion-autologous tumor systems (A, B, and C) being reported;

FIGS. 16A and 16B graphically displays the data recorded in Table 10 on the effects of unstimulated supernatants after separation into fractions with products M_{Γ}>50 kDa (HMW) and <50 kDa (LMW) compared to the combination of the ≦50 and ≧50 kDa fractions on the fractional inhibition of FasR expression of LS513 and SW480 cells, wherein FasR expression was determined by flow cytometry after 24 hours of exposure to supernatant fractions and is expressed as the mean channel fluorescence and the data represents the mean value of two experiments;

FIG. 17 graphically displays the data recorded in Table 11 on the effects of anti-CD3 mAb stimulated supernatants (25% v/v), IFN-γ (1,000 U/ml), TNF-α (1,000 U/ml), anti-IFN-γ antibody (10 μg/ml), and anti-TNF-α antibody (10 μg/ml) on the proliferation of LS513 cells;

FIG. 18 graphically displays the data recorded in Table 12 on the effects of anti-CD3 mAb stimulated supernatants, recombinant sFasL (rsFasL, 50 mg/ml), anti-FasR IgM antibody (10 μg/ml), and anti-FasL antibody (10 μg/ml), alone and in combination, on the proliferation of LS513 cells;

FIGS. 19A and 19B graphically display the data recorded in Table 13 on the effect of supernatant, >50 kDa fraction, and <50 kDa fraction on FasL mRNA (10 μg/ml) expression on SW480 cells at two different points in time,

FIGS. 20A and 20B graphically display the data recorded in Table 13 on the effects of supernatant, >50 kDa fraction, and <50 kDa fraction on FasL mRNA (10 μg/ml) expression on LS174 cells at two different points in time;

FIG. 21 depicts the effect of unfractionated supernatant and a >50 kDa fraction, and a <50 kDa fraction (25% v/v) on Bcl-2 and Bax protein expression, as determined by immunoblot, and of NF-kb and FasL mRNA expression, as determined by RT-PCT, wherein SW480 cells were exposed to supernatants for 24 hours after separation into fraction, where NT=no treatment;

FIG. 22 graphically displays the data recorded in Table 14 on the levels of cytokines of stimulated and unstimulated Cytokine C supernatants as determined by ELISA, wherein the data represent the mean±SD (standard deviation) for supernatants form 3 different activation-expansions;

FIG. 23 demonstrates the effect of stimulated Cytokine C supernatant on the cell cycle of LS513 cells as demonstrated by flow cytometry;

FIG. 24 graphically depicts the data set forth in Table 15 on the combined effects of CPT-11 and Cytokine C supernatants (SUP) on (A) FasR (CD95) expression as determined by flow cytometry, and (B) caspase-3 and caspase-8 activity as determined by colorimetric methods;

FIGS. 25-28 graphically depicts the data set forth in Tables 16 and 17 on the combined effects of irinotecan (CPT) and stimulated Cytokine C supernatants on colorectal cancer cells, where

FIGS. 26 and 28 display antiproliferative activity while

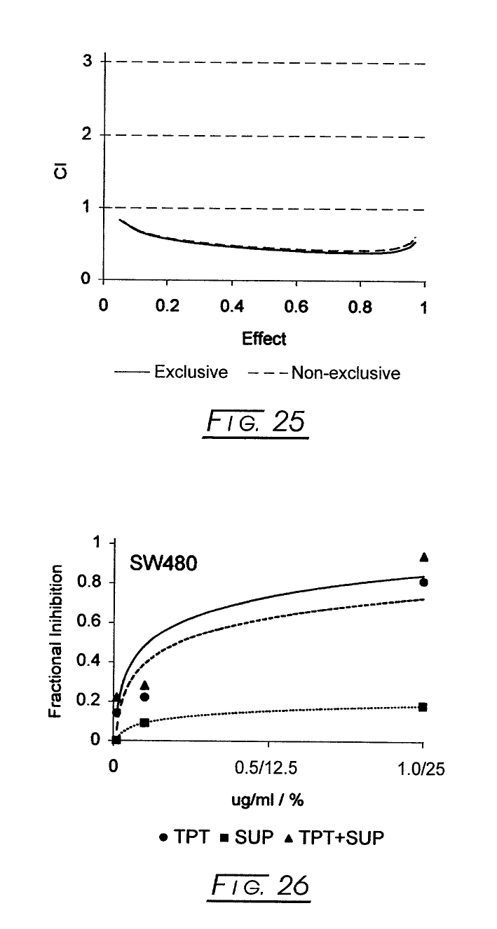

FIGS. 25 and 27 display the Cl plotted with the assumption that the agents are mutually non-exclusive or mutually exclusive in their mechanism of action, wherein the data reported represents the mean of three experiments;

FIG. 29-32 graphically depicts the data set forth in Tables 18 and 19 graphically depicts the data set forth in Tables 16 and 17 on the combined effects of topotecan (TPT) and stimulated Cytokine C supernatants on colorectal cancer cells, where

FIGS. 30 and 32 display antiproliferative activity while

FIGS. 31 and 33 display the Cl plotted with the assumption that the agents are mutually non-exclusive or mutually exclusive in their mechanism of action, wherein the data reported represents the mean of three experiments);

FIG. 33 graphically depicts the data set forth in Table 20 on the combined effects of TPT, SUP, TPT+SUP alone and with various blocking antibodies, wherein the data presented represents the fractional inhibition of triplicate samples;

FIG. 34 graphically depicts the data set forth in Table 21 on the time course of the chemosensitization effects of Factor C supernatants, wherein LS513 cells were cultivated with supernatants at 25% (v/v) for 6 hours, then resuspended in fresh media without supernatant, topotecan (1.0 μg/ml) added at 24, 48, 72, and 96 hours to the media and proliferation assessed 72 hours later, wherein the data represents the fractional inhibition of triplicate samples of cells exposed to topotecan alone (TPT) and those pre-treated with supernatant for 6 hours (SUP+TPT);

FIG. 35 graphically depicts the data set forth in Table 21 on the role of sFasL, TNF-α, and IFN-γ in the topotecan-supernatant interaction, wherein LS513 cells were cultured with topotecan (1μg/ml), supernatant (25% v/v), and anti-FasL, anti-TNF-α, and anti-IFN-γ antibodies (10 μg/ml), alone and in combination, wherein the data represent the mean factional inhibition of triplicate samples;

FIG. 36 graphically depicts the date set forth in Table 22 on the repression of HIV replication by the supernatant derived from OKT-3 anti-CD3 mAb stimulated HIV+ lymph node lymphocytes at different levels of OKT-3 at 20% supernatant concentration at Day 4;

FIG. 37 graphically depicts the date set forth in Table 22 on the repression of HIV replication by the supernatant derived from OKT-3 anti-CD3 mAb stimulated HIV+ lymph node lymphocytes at different levels of OKT-3 at 80% supernatant concentration at Day 4;

FIG. 38 graphically depicts the date set forth in Table 22 on the repression of HIV replication by the supernatant derived from OKT-3 anti-CD3 mAb stimulated HIV+ lymph node lymphocytes at different levels of OKT-3 at 20% supernatant concentration at Day 12;

FIG. 39 graphically depicts the date set forth in Table 22 on the repression of HIV replication by the supernatant derived from OKT-3 anti-CD3 mAb stimulated HIV+ lymph node lymphocytes at different levels of OKT-3 at 80% supernatant concentration at Day 12

FIG. 40 graphically depicts the data set forth in Table 26 on the effects of a range of concentrations of supernatants (volume/volume culture medium, v/v) collected from activated-expanded T-cells alone and in combination with tamoxifen at 10 μg/ml on the growth of SKBR3 cells, wherein the data represent mean±SD for three different experiments;

FIG. 41 graphically depicts the data set forth in Table 27 on the effects of supernatants from activated-expanded T cells after separation into fractions with products of >50 kDa and <50 kDa compared to the combination of the <50 and >50 kDa fractions (SUP or supernatant) on the proliferation of SKBR3 cells, alone and in combination with tamoxifen (TAM) at 10 μg/ml;

FIG. 42 is an SDS-PAGE gel under reducing conditions of an active fraction purified from a >50 kDa fraction of supernatants derived from activated-expanded CD4+ T cells showing a band at approximately 70 kDa (Factor C), wherein two other bands at 23 and 47 kDa also are apparent;

FIG. 43 graphically depicts the data set forth in Table 28 on the combined effects of a range of concentrations of Factor C and tamoxifen on the proliferation of ER-positive MCF-7 and BT414 cells and ER-negative SKBR3 cell lines;

FIG. 44 graphically depicts the data set forth in Table 29 on the effect of blocking antibody (50 μg/ml) to TNF-α, IFN-γ, RGF-β, and FasL on the combined effects of Factor C (5% v/v) and tamoxifen (TAM, 10 μg/ml);

FIG. 45 graphically depicts the data set forth in Table 30 on the combined effects of tamoxifen (TAM; 10 μg/ml) and Factor C (PF, 5% v/v) alone and in combination, on the growth (open bars, fractional inhibition) and induction of apoptosis (solid bars, CD95 mean cell fluorescence) in SKBR3 cells;

FIG. 46 depicts the effect of Factor C (PF, 5% v/v) and tamoxifen (TAM, 10 μg/ml) on the cell cycle of SKBR3 cells;

FIG. 47 graphically depicts the data set forth in Table 31 on the combined effects of tamoxifen (TAM, 10 μg/ml) and Factor C (PF, 5% v/v) on caspase-3 and caspase-8 in SKBR3 cells; and

FIG. 48 depicts the combined effects of unfractionated supernatants (25% v/v), Factor C (5% v/v), and tamoxifen (10 μg/ml) on protein kinase C alpha and delta, wherein lane 1=positive control, lane 2=no additions, lane 3=unfractionated supernatant, lane 4=tamoxifen, lane 5=unfractionated supernatant+tamoxifen, lane 6=Factor C, and lane 7=Factor C+tamoxifen.

The drawings will be described in detail below.

### DETAILED DESCRIPTION OF THE INVENTION

Three levels of data now are available. The initial data represents the infusion of the activated-expanded cells into patients having cancer, HIV-1 infection, and CFS. This is represented by U.S. Pat. Nos. 5,814,295 (cancer) and 6,093,381 (enhancement of chemotherapeutic agents against cancer), application Ser. No. 09/943,993 (HIV-1 and other viruses), and application Ser. No. 09/167,764 (CFS). The second level of data represents the activated-expanded cell supernatant separated into >50,000 and <50,000 dalton fractions in which the >50 k dalton fraction exhibited the greatest activity against cancer and HIV-1. The third level of data represents a Factor C of about 80 k dalton isolated from the >50 k dalton fraction, which purified Factor C exhibits activity against both cancer and HIV. The N-terminal sequences of the Factor C have not been matched in any library examined likely, then, is newly discovered. Attempts to locate the gene responsible for encoding such Factor C are underway presently.

Due to the unusual nature of a single Factor C exhibiting activity in such diverse disease types, its description necessarily also will involve both viral diseases and cancer. Even though these diseases have been though of as separate in the past, the Factor C disclosed herein provides a point of unification in the treatment and management of these diseases.

Oncology

Referring initially to the Factor C as it relates to cancer, the data reported herein examined the effects of the soluble products of tumor-reactive T cells on FasL-FasR interactions. The results of these data indicate that a complex combination of soluble factors that have been shown to modulate the growth of tumor cells, including those that have been reported to decrease tumor FasR, such as GM-CSF and TGF-β, and those that have been reported to upregulate tumor FasL, such as IFN-γ, are produced by tumor-reactive T cells. May, “Control of apoptosis by cytokines”, Advances in Pharmacology, 41:219-246, 1998; Schiller, et al., “Antiproliferative effects of tumor necrosis factor, gamma interferon and 5-fluorouracil on human colorectal carcinoma cell lines”, Int J Cancer, 46:61-6, 1990; Chu, et al., “The interactions of γ interferon and 5-flurouracil in the H630 human carcinoma cell line”, Cancer Res, 50:5834-5840, 1990; Lahm, et al., “Growth inhibition of human colorectal-carcinoma cells by interleukin-4 and expression of functional interleukin-4 receptors”, Int J Cancer, 59:440-447, 1994; Berdel, et al., “Stimulation of clonal growth of human colorectal tumor cells by Il-3 and Gm-CSF. Modulation of 5-FU cytotoxicity by GM-CSF”, Onkologie, 13a;437-443, 1990; and Uhm, et al., “Modulation of transforming growth factor-beta 1 effects by cytokines”, Immunological Investigations, 22:375-388,1993. The net direct effect of the soluble products on tumor cells, however, is to induce apoptosis by increasing FasR. Multiple levels of interaction that involve, but are not limited to, sFasL and FasR, are involved. In addition, the soluble factors released by tumor-reactive T cells do not appear to upregulate tumor FasL expression and a possible tumor “Fas counter-attack”. O'Connell, et al., “The Fas counterattack: Fas-mediated T cell killing by colon cancer cell expression Fas ligand”, supra; Tanaka, et al., “Fas ligand in human serum”, supra; and Shiraki, et al., “Expression of Fas ligand in liver metastases of human colonic adenocarcinomas”, supra.

T cells can secrete sFasL in response to tumors, and this sFasL has antiproliferative activity. As the antiproliferative activity was contained in the fraction of Mᵣ greater than 50,000, the active sFasL produced likely is the trimeric form, which has a Mᵣ of approximately 80,000. Fisher, “Apoptosis in cancer therapy: crossing the threshold”, supra. The role of sFasL production in the activity of activated lymphocytes, which are resistant to sFasL, has not been established. mFasL and cell-cell contact has been considered to be necessary for the induction of apoptosis, and recent studies have demonstrated conditions in which cells are sensitive to death by mFasL, but not by sFasL. Tanaka, et al., “Downregulation of Fas ligand by shedding”, Nature Med, 4:31-36, 1998; Suda, et al., “Membrane Fas ligand kills human peripheral blood T lymphocytes, and soluble Fas ligand blocks the killing”, J Exp Med, 186:2045-2050, 1997; Oyaizu, et al., “Requirement of cell-cell contact in the induction of Jurkat T cell apoptosis: the membrane-anchored but not soluble form of FasL can trigger anti-CD3-induced apoptosis in Jurkat T cells”, Biochem Biophys Res Commun, 238:670-675, 1997; Foote, et al., J lmmol, 157:1878-1885, 1996; and Thilenius, et al., Eur J Immunol, 27:1108-1114, 1997. SFasL-FasR complexes may be internalized by cells leading to downregulation of FasR. Tanaka, et al., “Downregulation of Fas ligand by shedding”, Nature Med, 4:31-36, 1998. SFasL-FasR complexes may be internalized by cells leading to the downregulation of FasR. Tanaka, et al., “Downregulation of Fas ligand by shedding”, Nature Med, supra. It has been speculated that CTL might secrete more sFasL monomer during the early stages of activation to prevent self-destruction by desensitizing their FasR and later switch to producing more mFasL, to increase their cytolytic activity towards target cells. The results reported herein would be consistent with the secretion of a more active, higher Mᵣ sFasL trimer by the lymphocytes generated by the methods utilized rather than the recombinant sFasL or sFasL purified from mouse cell tranformants expressing FasL applied in previous reports, which were of lower Mr. Tanaka, et al., “Downregulation of Fas ligand by shedding”, Nature Med, supra; and Suda, et al., “Membrane Fas ligand kills human peripheral blood T lymphocytes, and soluble Fas ligand blocks the killing”, J Exp Med, supra. The antiproliferative effects could not be completely blocked with anti-FasL antibody. Other members of the TNF family, such as TRAIL, could be playing a role. In addition, the results also suggest the possibility that other factors can enhance the sensitivity of the tumor cells, including cytokines of Mᵣ less than 50,000. Cytokines, such as IFNγ have been shown to augment Fas-mediated apoptosis. Morimoto, et al., “Overcoming tumor necrosis factor and drug resistance of human tumor cell lines by combination treatment with anti-Fas antibody and drugs or toxins”, Cancer Res, 53:2591-2596, 1993.

As has been previously reported, SW480 colorectal cancer cells express FasL. SW480 cells did not, however, secrete sFasL. The soluble products did not alter FasL expression of SW480 cells, nor of LS174T cells. There are conflicting reports regarding the functionality of the FasL produced by SW480 cells. Shiraki, et al., “Expression of Fas ligand in liver metastases of human colonic adenocarcinomas”, Proc Natl Acad Sci USA, 94:6420-6425, 1997; and Bohm, et al., “A modification of the JAM test is necessary for a correct determination of apoptosis induced by FasL”, J Immunol Methods, 217:71-78, 1998. The results reported herein would suggest that SW480 FasL is not functional. The sFasL ligand produced by SW480 cells existed at Mr 40,000 and did not induce apoptosis of FasL-sensitive Jurkat cells. The role of the “Fas counter-attack”, i.e., Fas-mediated T cell killing by tumor cell expressing FasL, in tumor escape mechanisms has not been established. Evidence that T cells are resistant to FasL in vivo, the pro-inflammatory effects of some systems, and technical issues regarding the assessments of FasL expression and activity, have led to questions regarding the role of tumor FasL in the escape from immune destruction. Bohm, et al., “A modification of the JAM test is necessary for a correct determination of apoptosis induced by FasL”, J Immunol Methods, id; Chappell, et al., “Human melanoma cells do not express Fas (Apo-1/CD95) ligand”, Cancer Res, 59:59-62, 1999; Kang, et al., “Fas ligand expression in islets of Langerhans does not confer immune privilege and instead targets them for rapid destruction”, Nat Med, 3:738-743, 1997; and Smith, et al., “Technical note: aberrant detection of cell surface Fas ligand with anti-peptide antibodies”, J Immunol, 160:4159-4160, 1998. The soluble products of tumor-reactive T cells, Factor C, did modulate other factors important in FasL-FasR interaction. Bcl-2 expression was increased; BAX was unaffected. A number of apoptotic pathways can be modulated by such Factor C secreted by tumor-reactive T-cells. Importantly, the induction of apoptosis by the Factor C of tumor-reactive lymphocytes does not appear to be related to p53, a central mediator of the cellular apoptotic response, as antiproliferative effects were observed in cells with mutated and normal p53. Apoptosis induced by chemotherapeutic drugs and irradiation is influenced by p53 expression. For example, apoptosis induced by treatment of etoposide or gamma irradiation resulted in apoptosis, associated with G2M arrest, in SW480 cells but not in LS174T cells. Arita, et al., “Induction of p53-independent apoptosis associated with G2M arrest following DNA damage in human colon cancer cell lines”, Japanese J Cancer Res, 88:39-43,1997.

A substantial proportion of the cells infused were CD4+ cells. Although CTL and NK cells can induce tumor cell apoptosis through Fas, granule-dependent cytolysis is the predominant killing pathway. Antitumor CD4+ cells may lack perform and other enzyme containing granules, and, thus, exert target cell killing through alternative mechanisms, such as FasL/FasR. Berke, “Killing mechanisms of cytotoxic lymphocytes”, Curr Opin Hematol, 4:32, 1997. Curti, et al. have recently reported tumor regressions using noncytolytic CD4+ T cells administered with system IL-2 and cyclophosphamide. Curti, et al, “Phase I trial of anti-CD3-stimulated CD4+ T cells, infusional inteleukin-2, and cyclophosphamide in patients with advanced cancer”, J Clin Oncol, 16:2752-2760, 1998.

Anti-CD3/IL-2 activated-expanded lymphocytes are in clinical trials. It also has been reported recently that the infusion of lymph node cells overexpressing FasL demonstrated antitumor activity in mice, whereas cells lacking FasL did not. Shimizu, et al., “Antitumor activity exhibited by Fas ligand (CD95L) overexpressed on lymphoid cells against Fas+tumor cells”, Cancer Immunol Imunother, 47:143-8, 1998. In addition, anti-CD3 mAb plus IL-2 have demonstrated antitumor activity in early-phase clinical trials. Sosman, et al., “Phase IB clinical trial of anti-CD3 followed by high-dose bolus interleukin-2 in patients with metastatic melanoma and advanced renal cell carcinoma: clinical and immunologic effects”, J Clin Oncol, 11:1496-1501, 1003; Hank, et al., “Clinical and immunological effects of treatment with murine anti-CD3 monoclonal antibody along with interleukin 2 in patients with cancer”, Clin Cancer Res, 1:481-491, 1995; Sosman, et al., “A phase IA/IB trial of anti-Cd3 murine monoclonal antibody plus low-dose continuous-infusion inteleukin-2 in advanced cancer patients”, J Immunother, 17:171-180, 1995; and Butler, et al., “Phase I/II study of low-dose intravenous OKT3 and subcutaneous interleukin-2 in metastatic cancer”, Eur J Cancer, 29A:2108-2113, 1993.

The mechanisms of tumor destruction in adoptive cellular therapy programs have not been established. The production of cytokines, such as TNF, in vitro has been a better predictor of the antitumor activity of adoptively transferred cells in vivo than their cytolytic activity in vitro, suggesting the possibility that he release of soluble factors may be important. Barth, et al., “Interferon γ and tumor necrosis factor have a role in tumor regression mediated by murine CD8+ tumor-infiltrating lymphocytes”, J Exp Med, 173:647-658, 1991. In addition, studies with tumor spheroids and xenografts appear to show complete destruction of tumors in the presence of incomplete penetration of the CTL and NK cells, which also supports the possibility that soluble factors are operational. Whiteside, et al., “Human tumor antigen-specific T lymphocytes and inteleukin-2-activated natural killer cells: comparison of antitumor effects in vitro and in vivo”, Clin Cancer Res, 4:1135-1145, 1998. Although the significance has not been clearly established, there are data that suggest hat the infiltration of colorectal tumors by lymphocytes confer a more favorable prognosis. Di Giorgio, et al., “The influence of tumor lymphocytic infiltration on long term survival of surgically treated colorectal cancer patients”, Int Sur, 77:256-60, 1993. Lymphocytes infiltrating colorectal cancers have low proliferative and cytolytic capacity, but have been shown to secrete normal levels of factors, such as IFN-γ. Bateman, et al., “Lymphocytes infiltrating colorectal cancer have low proliferative capacity but can secrete normal levels of interferon gamma”, Cancer Immunol Immunother, 41:61-7, 1995. Whether soluble Factor C is directly inhibiting cancer growth in the data reported is not yet determined. The systemic FasL based therapies evaluated to date have been prohibitively toxic in preclinical studies. Whereas locally applied FasL kills tumor cells very efficiently without systemic toxicity, intravenous administration of FasL induces lethal liver hemorrhage and hepatocyte apoptosis. Rensing-Ehl, et al., “Local Fa/APO-1 (CD95) ligand-mediated tumor cell killing in vivo”, Eur J Immunol, 25:2253-2258, 1995. Infusing cytokine/FasL-secreting lymphocytes, which have little clinical toxicity, offers an approach to increase the therapeutic index in the treatment of neoplastic diseases, particularly in light of the observation that normal cells uniformly appear to be resistant to sFasL. Tanaka, et al., “Downregulation of Fas ligand by shedding”, Nature Med, 4:31-36, 1998; and Strasser, et al., “Fas Ligand—caught between Scylla and Charbdis”, Nature Med, 4:21-22, 1998. Theoretically, the lymphocytes could traffic to tumor and release cytokines/FasL in a regulated, paracrine fashion. Finally, its has been reported recently that cytotoxic drugs can sensitize cancer cells to Fas-mediated apoptosis effected by cytolytic lymphocytes. Micheau, et al., “Sensitization of cancer cells treated with cytotoxic drugs to Fas-mediated cytotoxicity”, J Natl Cancer Inst, 89:783-789, 1997. This same tumor regression was observed (see U.S. Pat. No. 6,093,381) in patients subsequently treated with cytotoxic chemotherapy, including responses with 5-flurouracil (5FU) in patients who had previously progressed on 5FU. It is possible that the soluble Factor C produced by the tumor-reactive lymphocytes could be effectively combined with cytotoxic chemotherapeutics also.

Chemotherapeutic Enhancement in Oncology—Topoisomerase-I-active Drugs

Based upon such possibility, the purified Factor C was studied with respect to its effects when combined with cytotoxic chemotherapeutic agents. The data presented below also evaluates the combined effect of the soluble Factor C produced by TRL (tumor-reactive lymphocytes) on the cytotoxicity of camptothecins, an important new chemotherapeutic agent whose interactions with immune effectors have not been well characterized. Synergistic antiproliferative activity was observed.

The combination of the soluble products of TRL and topoisomerase-I-active drugs led to an increase in cleavable complex formation or stabilization. TRL soluble factors caused a slight increase in the activity of purified topoisomerase I as well as a slight increase in the amount of endogenous topoisomerase I produced by the tumor cells. This effect is not due to tumor cells being induced into S phase. In contrast, tumor cells exposed to the TRL supernatants accumulate in G1-G0. The combined effects of camptothecins and other cytotoxic chemotherapeutics have been extensively evaluated, and synergistic interactions have been reported. Kano, et al, “Effects of CPT-11 in combination with other anti-cancer agents in culture”, Int J Cancer, 50:604-610, 1992; Mattern, et al., “Synergistic cell killing by ionizing radiation and topoisomerase I inhibitor topotecan (SK&F 104864)”, Cancer Res, 51:5813-5815, 1991; and Anzai, et al., “Synergistic cytotoxicity with 2′-deoxy-5-azacytidine and topotecan in vitro and in vivo”, Cancer Res, 52:2180-2185, 1992. Antagonism, however, also has been observed. Cheng, et al., “Schedule-dependent cytotoxicity of topotecan alone and in combination chemotherapy regimens”, Oncol Res, 6:269-279, 1994; Chou, et al., “Computerized quantitation of synergism and antagonism of taxol, topotecan, and cisplatin against human teratocarcinoma cell growth: a rational approach to clinical protocol design”, J Natl Cancer Inst, 86:1517-1524, 1994; and Kaufmann, “Antagonism between camptothecin and topoisomerase II-directed chemotherapeutic agents in a human leukemia cell line”, Cancer Res, 51:1129-1136, 1991. Others found that the cytotoxic effects of topotecan and either antimetabolites, antimicrotubule agents, and DNA alkylating agents were less than additive, using median effect analyses. These observations were attributed to the decrease of cells entering S phase and diminished conversion of topotecan-stabilized topoisomerase I-DNA complexes into cytotoxic breaks. Kaufmann, et al., “Cytotoxic effects of topotecan combined with various anticancer agents in human cancer cell lines”, J Natl Cancer Inst, 88L734-741, 1996. Synergistic interactions between TRL soluble factors and camptothecins were observed in tumor cells with wild type p53, i.e., LS513, and those with mutated p53, ie., SW480. Apoptosis induced by immune effectors is considered to be independent of p53. The role of p53 in the regulation of camptothecin-induced apoptosis has not been fully characterized. Induction of apoptosis by topotecan appears to be largely independent of p53. Winter, et al., “Potentiation of CD95L-induced apoptosis of human malignant glioma cells by topotecan involves inhibition of RNA synthesis but not changes in CD95 or CD95L protein expression”, J Pharm Exp Ther, 286:1374-1382, 1998. Similar results have been observed with topotecan and irinotecan in the human myeloid leukemia HL-60, breast cancer MCF7, cervical HeLa, and pancreatic MIA cell lines.

TRL was activated and expanded in vitro with anti-CD3 mAb and Il-2. They produce a variety of growth-inhibitory and growth-stimulatory factors in response to tumors. Kim, et al., “Expansion of mucin-reactive lymph node lymphocyte subpopulations form patients with colorectal cancer”, Cancer Biother, 10:115-123, 1995; Triozzi, et al., “Adoptive immunotherapy using lymph node lymphocytes localized in vivo with radiolabeled monoclonal antibody”, J Natl Cancer Inst, 87:1180-1181, 1995; Triozzi, et al., “Identification of tumor-reacting lymph node lymphocytes in vivo using radiolabeled monoclonal antibody”, Cancer 1994; 73:580-589; Kim, et al., “Cellular immunotherapy of patients with metastatic colorectal cancer using lymph node lymphocytes located in vivo with radiolabeled monoclonal antibody”, Cancer, 86;22-30, 1999; and Triozzi, et al., “Induction of Fas-mediated apoptosis by the soluble factors secreted by tumor-reactive T-cells”, submitted. FasR upregulation and blocking studies with anti-FasL antibody suggest the possibility that the Fas system may be involved. The role of soluble FasL in the antitumor activity of activated lymphocytes is not known. Its has been suggested recently that the soluble form of Fas ligand, which can be released by activated T cells, not only induces apoptosis less potently than insoluble membrane-bound FasL, but can antagonize the apoptosis-inducing activity of membrane-bound FasL and downregulate FasR. Tanaka, et al., “Downregulation of Fas ligand by shedding”, Nature Med, 4:31-36, 1998. Although chemotherapy-induced apoptosis may not be dependent on the FasL-FasR interaction, there is evidence that the Fas system may play a role in the activity of cytotoxic drugs in some situations. It has been reported recently that a 27 kDa sFasL is constitutively secreted by LNCaP prostrate cancer cells in vitro. Liu, et al., “Fas ligand is constitutively secreted by prostate cancer cells in vitro”, Clin Cancer Res, 4:1803-1811, 1998. There is evidence that the upregulation of FasR in the presence of FasL accounts, in part, for the cytotoxicity of mitoxantrone in this cell line. Other cytokines previously reported to modulate topoisomerase I activity, such as TNF-α, IFN-α, and IL-1α, do not appear to play a role in the interactions observed, nor does IFN-γ, a cytokine that can enhance the sensitivity of the tumor cells to Fas-mediated apoptosis. Morimoto, et al., “Overcoming tumor necrosis factor and drug resistance of human tumor cell lines by combination treatment with anti-Fas antibody and drugs or toxins”, supra.

It has been difficult to develop effective combinations of cytokines and chemotherapeutics. In addition to the limitations noted above, the clinical toxicity of the high concentration of cytokines necessary, cytokine combinations in particular, have limited biochemotherapy approaches. Stein, et al., “Modulation of mdrl expression by cytokine in human colon carcinoma cells: an approach for reversal of multidrug resistance”, Br J Cancer, 74:1384-1391, 1996; Walther, “Influence of cytokines on mdrl expression in human colon carcinoma cell lines: increased cytotoxicity of MDR relevant drugs”, J Cancer Res Clin Oncol, 120:471-478,1994; and Borsellino, et al., “Combined activity of interleukin-1 alpha or TNF-alpha and doxorubicin on multidrug resistance cell lines: evidence that TNF and DXR have synergistic antitumor and differentiation-inducing effects”, Anticancer Res, 14:2640-2648, 1994. Intratumoral and locoregional treatments, including transduction of cytokine genes, have been considered, but have the obvious limitation of delivery to metastatic tumor. Stein, et al., “Reversal of multidrug resistance by transduction of cytokine genes into human colon carcinoma cells”, J Natl Cancer Inst, 88:1383-1392, 1996.

U.S. Pat. No. 5,814,295 demonstrates antitumor activity in clinical trials of cytokine-producing, noncytolytic TRL. Tumor regression in patients treated with these TRL and cytotoxic chemotherapy also are reported in U.S. Pat. No. 6,093,381. Others have recently reported tumor regressions using noncytolytic, cytokine-secreting peripheral blood CD4+ T cells administered with system IL-2 and cyclophosphamide. Curti, et al, “Phase I trial of anti-CD3-stimulated CD4+ T cells, infusional inteleukin-2, and cyclophosphamide in patients with advanced cancer”, supra. In addition, anti-CD3 mAb plus IL-2, the agents used to activate and expand TRL ex vivo (and also noncytolytic CD4+ T cells) have demonstrated antitumor activity in clinical trials. Hank, et al., “Clinical and immunological effects of treatment with murine anti-CD3 monoclonal antibody along with interleukin 2 in patients with cancer”, supra. Antitumor activity has been achieved with manageable toxicity. The results reported herein suggest that these approaches that result in the production of multiple-cytokines could be effectively combined with topoisomerase-I-active drugs.

Chemotherapeutic Enhancement in Oncology—Tamoxifen

Tamoxifen, a non-steroidal anti-estrogen, is an important agent in breast cancer therapy and chemoprevention. Although tamoxifen is believed to inhibit tumor growth primarily through competing with estrogen for estrogen receptor (ER) binding, the mechanism of its antitumor activity remains unclear. Tamoxifen has demonstrated in vitro antitumor activity against many ER-negative cancer cell lines, including non-breast cancers, as well as clinical antitumor activity in some patients with ER-negative breast cancers. Tamoxifen has a variety of other effects that may play a role in its antitumor activity. This has been reviewed by Friedman, “Recent advances in understanding the molecular mechanism of tamoxifen action”, Cancer Invest, 16:391-396, 1998. These include inhibition of protein kinase C (O'Brian, et al., “Inhibition of protein kinase C by tamoxifen”, Cancer Res, 45:2462-2465, 1985), inhibition of phospoholipase C (Freidman, “The anti-tumor agent tamoxifen inhibits breakdown of polyphosphoinositids in GH₄C₁ cells”, J Pharmocol Exp Ther, 271:617-623, 1993), and stimulation of phosphoinositide kinase (Friedman, “Tamoxifen and vanadate synergize in causing accumulation of polyphosphoinositide in GH₄C₁ membranes”, J Pharmacol Exp Ther, 267:617-623, 1993). Tamoxifen also has been shown to inhibit calmodullin (MacNeil, et al., “Antiproliferative effects on keratinocytes of a range of clinically used drugs with calmodulin antagonist activity”, Br J Dermatol, 128:143-150, 1993) and stimulate transforming growth factor-β (RGF-β ) (Benson, et al., “Modulation of transforming growth factor β expression and induction of apoptosis by tamoxifen in ER positive and ER negative breast cancer cells”, Br J Cancer, 72:1441-1446, 1995). Morphological changes and DNA fragmentation consistent with apoptosis also has been reported. Treon, et al., “Anti-estrogens induce apoptosis of multiple myeloma cells”, Blood, 92:1749-1757, 1998. Tamoxifen-induced apoptosis in breast cancer cell relates to down-regulation of bcl-2, but not bax and bcl-XL, without alteration of p53 protein levels. Zhang, et al., “Tamoxifen-induced apoptosis in breast cancer relates to down-regulation of bcl-2 but not bax and bcl-X_{L}, without alteration of p53 protein levels”, Clin Cancer Res, 5:2751-2977, 1999. Fas, an important mediator of apoptosis in the TFN family of receptors, may be involved. Pan, et al., “Apoptosis and tumorigenesis in human cholangiocarcinoma cells. Involvement of Fas/APO-1 (CD95) and calmodulin”, Am J Pathol, 155:193-203, 1999. Tamoxifen induced apoptosis in Fas-positive cholangiocarcinoma cells, which were ER negative, but not in Fas-negative cells. Furthermore, apoptosis induced by tamoxifen in Fas-positive cells was blocked by inhibitory Fas antibody.

Several studies have indicated that tamoxifen can interact synergistically with immune effector molecules and cells. Tamoxifen can sensitize tumor cells for killing by NK, lymphokine activated killer, and cytolytic T lymphocytes (CTL). Baral, et al., “Enhancement of natural killer cell mediated cytotoxicity by tamoxifen”, Cancer, 75:591-599, 1995; and Baral, et al., “Combination immunotherapy of the p815 murine mastocytoma with killer cells, IL-2 and anti-estrogens”, Anticancer Res, 17:3653-3658, 1997. Synergistic cytotoxic effects of TNF, interferon-α (IFN-α), and IFN-γ with tamoxifen have been demonstrated in ER-positive and ER-negative cells. Tiwari, et al., “Augmentation of cytotoxicity using combinations of interferons (types I and II), tumor necrosis factor-alpha, and tamoxifen in MCF-7 cells”, Cancer Lett, 61:45-52, 1991; Matuso, et al., “Synergistic cytotoxic effects of tumor necrosis factor, interferon-gamma and tamoxifen on breast cancer cells lines”, Anticancer Res, 12:1575-1579, 1992; and Iwasaki, et al., “Inhibitory effects of tamoxifen and tumor necrosis factor alpha on human glioblastoma cells”, Cancer Immunol Immunother, 40:228-234,1995.

The antitumor effects of Factor C with tamoxifen can inhibit growth of breast cancer cells and enhance the antitumor activity of tamoxifen. Synergistic antiproliferative interactions were observed with Factor C and tamoxifen in ER-positive and ER-negative breast cancer cell lines. The enhanced antiproliferative activity was associated with morphologic evidence of apoptosis; an increase in cell in G1/G0, expression of Fas, in the activity of caspase-3 and casapse-8; and a decrease in protein kinase C levels. Blocking antibody to TNF-α, TGF-β, and IFN-γ, and Fas ligand (FasL) had no effect on the activity of Factor C. The results reported herein are indicative of a novel method of enhancing the effects of tamoxifen.

HIV

Turning now to HIV-1, a FACTOR was identified, which factor is produced by CD4+ cells, then suppresses HIV-1 replication in naturally and acutely infected CD4+ cells in a dose dependent manner. This factor blocks HIV-1 replication by inhibiting LTR-driven transcription. It does not inhibit CD4+ cell proliferation. This factor shares several features with CAF as described by Levy, et al. However, CAF is only observed with CD8+ cells, and not with CD4+ cells, and CAF has a Mr of less than 30. It also lacks identity with chemokines and cytokines that have been reported to directly inhibit HIV-1 in CD4+ cells.

The active factor can be derived from lymph nodes and peripheral blood of HIV-1 infected patients, from cancer patients, and from peripheral blood of normal volunteers. Expansion-activation used the capacity of anti-CD3 mAb to mimic the pathways of T-cell activation and the capacity of IL-2 to expand multiple T-cell subpopulations. Cells were cultured in serum-free conditions using a media designed to maintain the viability of APC, i.e., macrophages and dendritic cells, while also providing adequate nutrition of the expanding lymphocytes. A preferred cell culturing technique comprehends culturing the cells with 10 ng/ml of anti-CD3 monoclonal antibody and 100 U/ml of human recombinant IL-2 in serum-free medium in 5% CO₂ in humidified air at 37° C. Cells then are counted and resuspended at day 3 to 4, depending upon growth. A small aliquot of cells is removed each time cells are counted and/or split. Day 10 cells are harvested by centrifugation (250× g, room temperature, 6 minutes) in 50 ml tubes. The pelleted cells then are resuspended at 1.5×10⁶/ml and put into T-75 flasks pre-coated with anti-CD3 mAb, with and with out anti-CD28 mAb, at a final volume of 200 ml per flask with 100 ng/ml of each antibody. Cells are cultured for 24 hours at 37° C. in 5% CO₂, and supernatants are collected by centrifugation at 400× g for 10 minutes.

For Factor C purification, 2 liters of supernatant is prepared for column chromatography by adding phenylmethyl sulfonyl fluoride and glycerol to 0.1% weight/volume. The supernatant is re-centrifuged for 30 minutes at 100 g to remove remaining particulates. The supernatant then is loaded onto a 120-ml bed volume Con-A Sepharose column at 10 ml/min. Unbound protein is rinsed off with 2 bed volumes of PBS, pH 7.2, and bound protein is eluted with 2 bed volumes of 8% α-D-mannopyranoside in phosphate buffered saline. Peak fractions are pooled and dialyzed against 10 volumes of 20 mM Hepes buffer 0.1 % glycerol, pH 8.2, overnight at 4° C., using SpectrumPor CE Membrane with a 50,000 molecular weight cut-off. This is applied to DEAE Sepharose equilibrated with 20 mM Hepes, pH 8.2. Bound protein is eluted with a step gradient of 200 and 500 mM NaCl in Hepes buffer. Protein is concentrated using Millipore Ultrafree centrifugal filter devices, 50,000 molecular weight cut-off, and re-suspended in RPMI with 10% fetal calf serum for bioassay.

Several studies have demonstrated that HIV-1-specific T-cell lines can be expanded by nonspecific stimulation with anti-CD3 mAb and IL-2 without the need for re-exposure to viral antigen. Although soluble a nti-CD3 is a stimulus for HIV-1 production, it also is a stimulus for chemokine release. Cocchi, et al., “Identification of RANTES, MIP 1α and MIP 1β as the major HIV-suppressive factors produced by CD8+ cells”, supra. Relatively low concentrations of anti-CD3 that would provide both immobilized anti-CD3, by association with APC, and soluble anti-CD3 mAb, were used.

Apoptosis also is postulated to be involved as an anti-viral immune mechanism by mediating the death of infected cells before viral replication has occurred. The FasL-FasR interaction is an important regulator of T cell apoptosis and could potentially act as a potent anti-viral immune mechanism against T-cell tropic viruses, such as HIV-1. Inhibition of T-cell apoptosis in vitro enhances the production of HIV and thereby facilitates persistent infection. Reconstitution of FasL activity with an anti-FasR Ab mimics the activity of membrane bound FasL has been shown recently to inhibit HIV-1 production in vitro. Walker, et al., “CD8+ lymphocytes can control HIV infection in vitro by suppressing virus replication”, supra.

Because of genetic mutability and the emergence of drug-resistant variants, advanced HIV-1 infection and related immunosuppression are unlikely to be effectively controlled with anti-retroviral agents alone. Much recent attention has focused on the soluble factors released by immune cells. The role of T-cell-derived suppressive factors in the control of HIV-1 infection has not yet been established. The C-C chemokines may play a prominent role in the control of HIV-1. RANTES, MIP-1α, and MIP-1β have been shown to potently suppress acute infection by macrophage-tropic strains; and it has recently been demonstrated that their G-protein-coupled receptor, CC CKR5, is a fusion cofactor for macrophage-tropic strains, defects in which account for resistant of some individuals to HIV-1. Cocchi, et al., “Identification of RANTES, MIP 1α and MIP 1β as the major HIV-suppressive factors produced by CD8+ cells”, supra; Alkhatib, et al., “A RANTES, MlP-1α, MIP-1β receptor as a fusion cofactor for macrophage-tropic HIV-1“, science, 272:1955-7958, 1996; Liu, et al., “Homozygous defect in HIV-1 co-receptor accounts for resistance of some multiply-exposed individuals to HIV-1 infection”, Cell, 86:367-377, 1966; and Samson, et al., “Resistance to HIV-1 infection in Caucasian individuals bearing mutant alleles of the CCR-5 chemokine receptor gene”, Nature, 383:722-725, 1996. Whether suppressive factors can be practically applied to the therapy of HIV-1 infection, advanced infection in particular, also is controversial. Chemokines exert pro-inflammatory effects that can be beneficial and detrimental, depending upon a variety of factors, including the nature of the responding cell and the concentration of the chemokine. Kunkel, et al., “Chemokines and their role in human disease”, Agents Actions Supplement, 46:11-22, 1995.

Factor C

Now that each of the disease areas have been discussed in detail, an additional piece of data necessary to connect Factor C of cancer and HIV-1 resides in an expected negative control for HIV-1 wherein a cancer patient supernatant was used. Unexpectedly, such cancer supernatant inhibited HIV-1 replication. Further research revealed that Factor C responsible for the HIV-1 replication is the same Factor C responsible for the cancer responses reported herein.

With respect to such Factor C, the N-terminal sequence of the novel Factor C is as follows: SER/GLY PRO ALA PRO MET MET LYS PHE PHE THR THR LYSNAL (SEQ. ID NO.: 5).

Factor C has a molecular weight of about 80,000 daltons, is heat stable, has an amino acid sequence that is absent from the National Center for Biotechnology Information database, and whose amino acid sequence is not homologous to TNF family ligands. Factor C is derived from CD4 cells in a much greater quantity than from CD8 cells, and is derived from lymph cells in a greater quantity than from PBL cells. A double activation and expansion (activation-expansion) process using immobilized and soluble anti-CD3 mAb makes such Factor C. Factor C appears to inhibit transcription in virally-infected and tumor cells, and stimulates the proliferation of normal lymphocytes. Factor C exhibits synergistic activity with topoisomerase 1, topoisomerase 11, microtubule, and thymidylate synthetase active agents; is responsible for the synergistic induction of apoptosis; its effect is not secondary to enhanced cell cycling; inhibits the anti-apoptotic factor, NFKB implicated in chemoresistance; enhances uptake of doxorubicin in multi-drug resistant cells, increases covalent topoisomerase I-DNA complexes with topoisomerase I active drugs; and decreases thymidylate synthetase transcription in combination with 5-flurouracil. Factor C with the hormonal agent, tamoxifen, is responsible for the synergistic induction of apoptosis and exhibits synergism in estrogen-receptor-negative and estrogen receptor-positive cell lines.

Factor C may be more than one molecule. It may be thought of as a “cytokine” since it is produced by lymphocyte cells or as a “lymphokine”. Regardless, Factor C has been demonstrated to have anti-viral activity as well as anti-tumor activity. Factor C is produced by the activation-expansion of CD4 lymphocyte cells in the presence of anti-CD3 mAb and IL2. The resulting supernatant is subject to fractionation to recover the fraction having a molecular weight of above about 50,000 (50 k) daltons. Such “high” molecular weight supernatant fraction has been demonstrated to exhibit anti-viral activity as well as anti-tumor activity. Factor C is contained in such high molecular weight supernatant fraction.

Factor C itself is a component of the high molecular weight supernatant fraction, which has a molecular weight of about 70,000-80,000 daltons. As noted above, this band may be composed of more than one component. Regardless of its precise composition, such band, or Factor C, has been demonstrated to exhibit anti-viral activity against HIV, herpes simplex virus, and Coxsackie virus; and anti-tumor activity against adenocarcinoma cancers. Based on the work reported in the cross-referenced applications, it is believed that Factor C has applicability in the treatment of immune mediated diseases (of which HIV is an example) in both animals and humans. While some of these diseases are bacterial (e.g., tuberculosis) and some are of unknown cause (autoimmune diseases, e.g., rheumatoid arthritis), most such immune mediated diseases are viral induced and result from persistent and acute infections, including latent infection (e.g., human herpes virus), chronic infections (e.g., “old dog encephalitis” following canine distemper virus (CDV) infection or lymphocyteic choriomeningitis in mice), and slow infections (both lentiviruses including HIV, feline immunodeficiency virus (FIV), and simian immunodeficiency virus (SIV); and a group of unclassified agents which cause subacute spongioform encephalopathies including Cruetzfeld-Jakob disease, Kuru, and Mad Cow Disease). Such immunosuppressive or chronic diseases that lead to an immunosuppressed state in the host (both human and animal) should be treatable in accordance with the precepts of the present invention including, for example, HIV, tuberculosis, measles, dengue fever, malaria, hepatitis (chronic), leprosy, rheumatoid arthritis, multiple sclerosis, canine distemper virus, and the like.

Chronic and acute viruses are classified as being DNA viruses or RNA viruses, enveloped and non-enveloped. RNA viruses are exemplified by, for example, picornaviruses, togaviruses, paramyxoviruses, orthomyxoviruses, rhandoviruses, reoviruses, retroviruses, bunyaviruses, coronaviruses, and arenaviruses. DNA viruses are typified by panoviruses, papoviruses, adenoviruses, herpesviruses, and poxviruses. For more information on viruses, reference is made to the following texts: Fenner, et al., Veterinarian Virology, 2nd Edition, Academic Press, New York, New York (1993); Mims, et al., Viral Pathogenesis and Immunology”, Blackwell Scientific Publications, London, England (1984); Virology, B. N. Field, Editor, Raven Press, 3rd edition; Shulman, et al., The Biological and Clinical Basis of Infectious Diseases, 5th edition, W.B. Saunders Co. (1997), the disclosures of which are expressly incorporated herein by reference.

While the invention has been described with reference to certain advantageous embodiments, those skilled in the art will understand that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In this application most units are in the metric system and all amounts and percentages are by weight, unless otherwise expressly indicated. Also, all citations referred herein are expressly incorporated herein by reference.

### EXAMPLES

### Example 1

### HIV Data

### Experimental

Reagents

OKT3 (Orthocione, Ortho Pharmaceutical Corporation, Raritan, N.Y.), recombinant Human IL-2 (Proleukin, Chiron, Emeryville, Calif.), RPMI-1640 medium supplemented with 10% fetal bovine serum (Gibco/BRL, Grand Island, N.Y.), and a serum-free medium, Macrophage SFM (Gibco/BRL) containing antibiotics were utilized. HIV-1 stock used for HIV-1 suppression assays was privately supplied by Dr. Michael Para.

Cell Culture and Factor Production

Cells were cultured in a range of concentrations of OKT-3 and IL-2 in serum-free medium in 5% CO₂ in humidified air at 37° C. Cells then were counted and resuspended every 3-4 days depending upon growth. A small aliquot of cells was removed each time cells were counted and/or split. Day 10 cells were harvested by centrifugation (250× g, room temperature, 6 min) in 50 ml tubes. The pelleted cells then were resuspended at 1.5×10⁶/ml. Harvested cells were tested unseparated or separated into purified CD8⁺ and CD4⁺ cells using anti-CD8 coated plastic flasks (Cellector-8, AIS, Santa Clara, Calif.) according to the recommendation of the manufacturer. CD4⁺ cell contamination of the CD8⁺ cells was less than 2.0% (as determined by flow cytometry). The cells then were put into T-175 flasks pre-coated with OKT3 with and without anti-CD28 antibody at a final volume of 200 ml per flask with 100 ng/ml of each antibody. Cells were cultured for 24 hours at 37° C. in 5% CO₂ and supernatants were collected by centrifugation at 400× g for 10 minutes.

Factor Purification

Two liters of supernatants were prepared for column chromatography on ConA Sepharose by adding phenylmethyl sulfonyl fluoride and glycerol to 0.2% weight/volume. The supernatant was re-centrifuged for 30 minutes at 1,000 g to remove remaining particulates. The supernatant then was loaded onto a 120-ml bed volume of ConA Sepharose column (Pharmacia) at 10ml/min. Unbound protein was rinsed off with 2 bed volumes of PBS, pH 7.2, and bound protein was eluted with 2 bed volumes of 8% α-D-mannopyranoside in phosphate buffered saline. Peak fractions were pooled and dialyzed against 10 volumes of 20 mM Hepes buffer 0.1% glycerol, pH 8.2, overnight at 4° C., using SpectrumPor CD Membrane with a 50,000 molecular weight cut-off (Spectrum Medical Industries, Houston, TX). This was applied to DEAE Sepharose (Pharmacia) equilibrated with 20 mM Hepes, pH 8.2. Bound protein was eluted with a step gradient of 200 and 500 mM NaCl in Hepes buffer. Protein was concentrated using Millipore Ultrafree centrifugal filter devices, 50,000 molecular weight cut-off, and re-suspended in RPMI with 10% fetal calf serum for bioassay. Unless otherwise stated, the supernatant was derived from a cancer-free, HIV-free patient, as the data determined that the factor was present in all patients tested.

HIV Staining of CD4 Cells

Normal human PBMC were isolated from 60 ml EDTA treated blood using Ficoll-Hypaque (Sigma). CD8 cells were depleted using immunomagnetic beads (Dynabeads M450, Dynal) as per manufacturer's instructions. CD8 depleted cells then were cultured with 2 mg/ml PHA (Pharmacia) in RPMI 20% FCS for 4 days at 37° C. and 5% CO₂. The resulting CD4 blast (5×10⁵) were infected with 100 μl HTLV-lllmn (NIH RRRP) in 24 well plates with and without dilution of semi-purified factor; 300 μl total volume for 2 hours at 37° C. Wells then were filled to 1 ml with RPMI 105 FCS and 180 I.U. recombinant IL-2 (Chiron). Cells were maintained at 1 to 2×10⁶ cells/ml by addition of fresh media with IL-2 every 2 days. Wells treated with factor were maintained at the initial concentration of factor throughout the duration of the culture. Cells were stained for HIV-1 core antigen of days 3, 6, and 8, after infection using Coulter Clone KC57-FITC labeled antibody following the manufacturer's protocol. Staining was analyzed on a Coulter Epics Elite flow Cytometer.

Flow Cytometry

Cell surface marker analysis was performed on an Eics Elite cytofluorograph using fluoresceinated or phycoerythrinated mAb as previously described. Triozzi, et al., “Identification of tumor-reacting lymph node lymphocytes in vivo using radiolabeled monoclonal antibody”, Cancer 1994; 73:580-589. The following mAbs were used: anti-CD3 (Leu4), CD4 (Leu3a), CD8 (Leu2a), 11b (Leu15), CD14 (LeuM3), CD44, Cd56 (Leu19), CD45RO (Leu45RO), CD45RA (Leu45RA), and anti-HLA-DR (all supplied from Becton-Dickinson, San Jose, Calif.); anti-CD30 (DAKO Corporation, Carpinteria, Calif.); and anti-CDw60 (PharMingen, San Diego, Calif.).

Immunoblot Analysis

CD4 cells exposed to fractions from lymphocyte culture supernatants were harvested and suspended in SDS buffer. Cell extracts were boiled for 10 min and chilled on ice. Total proteins from CD4 cells were separated on a 0.8% SDS-PAGE and electrophoretically transferred to a PVDF membrane. The membranes were incubated with appropriate polyclonal antibodies (anti-rabbit IgG bcl-2 or Bax) (Calbiochem) for 6-8 hours and washed with TTBS and incubated with secondary antibody conjugated with alkaline-phosphatase. The signal then was detected with BCPIP. NBT (5-bromo-4-chloro-3-indolyl-phosphate/nitro blue tetrazolium) color substrate in an alkaline phosphatase buffer (100 mM Tris-HCl (pH 9.5), 100 mM NaCl, 5 mM MgCl₂).

Reverse Transcription Polymerase Chain Reaction

The expression of HIV-1 (gag gene), FasL, Bcl-2, and Bax was determined by reverse transcription of total RNA followed by a PCR analysis (RT-PCR). PCR primers were designed using primer analysis software (Oligo, National Biosciences, Inc., Plymouth, Minn.) and obtained from Stratagene (La Jolla, Calif.) as previously described. Triozzi, et al., “Phenotypic and functional differences between human dendritic cells derived in vitro from hematopoietic progenitors and from monocytes/macrophages”, J. Leukoc. Bio., 1997: 61:600-608. Approximately 10⁶ cells were lysed in Trizol reagent (Life Technologies) and RNA was isolated according to the manufacturer's instructions. cDNA was synthesized by extension with random primers with 200 units of Super script 11 reverse transcriptase (Life Technologies). The reaction mixture contained 1 μg of total RNA in a final volume of 20 μl. To determine the purity of RNA RT reactions also were performed on RNA samples without the enzyme and the samples were used in PCR reactions. The 2 μl cDNA was used in a 20 μl reaction volume containing all four dNTPs (10 μM), nM MgCl₂ and 2.5 units of Taq polymerase (Life Technologies) and each primer at 1 μM. The amplification cycles were 94° C. for 30 seconds, 60° C. for 2 minutes (×30). Primers used for amplification were FasL sense primer corresponding to nucleotides 110-131 (5′-TCC TTG ACA CCT CAG CCT CTA-3′) (SEQ. ID No.: 1), and antisense primer complimentary to nucleotides 713-693 (5′-CCT CAC TCC AGA AAG CAG GAC-3′) (SEQ. ID No.: 2). The amplified products from the PCR reaction were separated on 1% agarose gel and visualized by ethidium bromide staining. To detect the levels of NF-Kβ, the amplification cycles were 94° C. for 30 sec, 60° C. for 30 sec, and 72° C. for 1 minute (x 30). Primers used for amplification were NF-Kβ sense primer corresponding to nucleotides 1792-1812 (5′-CTT TCT GCT GCG GGT AGG TG-3′) (SEQ. ID. NO.: 3), and antisense primer complimentary to nucleotides 2707-2687 (5′-GCT TGT CTC GGG TTT CRG GA-3′) (SEQ. ID. No.: 4). The amplified products from the PCR reaction were separated on 1% agarose gel and visualized by ethidium bromide staining.

Acute Infection

The effects of culture supernatants on HIV-1 production was assessed using previously described methods. Mackewicz, et al., “CD8+ cell anti-HIV activity: nonlytic suppression of virus replication”, Aids Res Hum Retrovirus, 1992; 8:629-64. Peripheral Blood CD4 T-cells were isolated from an uninfected donor using negative selection (Human T cell CD4 Subset Column Kit, R&D Systems, Inc., Minneapolis, Minn.). These purified CD4+ cells were activated with 10 ng/ml OKT3 and grown in RPMI-1640 medium supplemented with 10% fetal bovine serum and 100 IU IL-2. Cells were maintained between 0.5 and 2×10⁶/ml by adding fresh complete medium weekly. CD4+ T cells (5×10⁵/ml/well) were added to a 24-well plate containing complete medium with 20% or 805 of supernatants from lymph node cell expansion cultures from HIV-1-infected donors (n=2) and a HIV-1 negative donor. All wells, except the “no virus” control wells, were infected with HIV-1 culture supernatant known to contain sufficient HIV-1 to infect lymphocyte cultures at he proportions used. Supernatants were collected from the 24-well plate at twice weekly intervals. At each collection, the same proportion of supernatants from the original expansion cultures was added. Day 4 and Day 12 supernatants from the 24-well plate were analyzed by quantitative ELISA for HIV-1 p24 antigen (Coulter, Hiahlea, Fla.) as were the supernatants from the original expansion cultures. The amount of p24 produced was calculated by subtracting the p24 present in the 20% or 80% of the original expansion culture supernatant from the p24 detected in the 24-well plate. The p24 produced in control wells with complete medium along, the “virus positive” control, was compared to the p24 produced in test wells containing medium with 20% or 80% expansion culture supernatants. Data are presented as “% suppression [(virus positive control p24−test p24)/virus positive control p24]×100.”

Long-terminal Repeat (LTR) Driven Replication

LTR-driven replication was assessed using HeLA-CD4-LTR-β-gal cells, which were obtained through the AIDS Research and Reference Reagent Program, Division of AIDS, NIAID, NIH from Dr. Michael Emerman. The assay was performed as described by Kimptom, et al., “Detection of replication competent and pseudotyped HIV with a sensitive cell line based on activation of an integrated beta-galactasidase gene”, J Virol, 66:2232-2232, 1992.

### Results

Soluble Factor(s) Produced By Activated-Expanded CD4 Cells Suppress HIV-1 RNA and Protein In Naturally Infected Or Acutely Infected CD4 Cells

Cells were activated and expanded from lymph nodes and peripheral blood of HIV-1-infected and cancer patients and from the peripheral blood of healthy volunteers. The activated-expanded T cells were separated into CD4 and CD8 fractions using immunobead techniques prior to re-stimulation with anti-CD3. Supernatants then were added to HIV-1 -infected lymph node cells derived from HIV-1-infected patients and cultured in IL-2. Supernatants from both the CD4 and CD8 fractions produced soluble factors that inhibited HIV-1 mRNA expression as determined by PCR, in FIG. 1. The activity observed above using cells from lymph nodes and peripheral blood of IV-1 infected patients and in cancer patients.

Supernatants from CD4 and CD8 cells also were assessed in acute infection assays of CD4 cells in which p24 production was evaluated. The data recorded is set forth below in Table 1 and in FIG. 2.

TABLE 1

| | % Suppression |
|---|---|
| Experiment 1 | |
| Control | 4 |
| CD4 Unfractionated | 33 |
| CD4 Mr<50 | 10 |
| CD4 Mr>50 | 44 |
| CD8 Unfractionated | 26 |
| CD8 Mr<50 | 11 |
| CD8 Mr>50 | 27 |
| Experiment 2 | |
| Control | 5 |
| CD4 Unfractionated | 38 |
| CD4 Mr<50 | 5 |
| CD4 Mr>50 | 49 |
| CD8 Unfractionated | 31 |
| CD8 Mr<50 | 4 |
| CO8 Mr>50 | 34 |
| Experiment 3 | |
| Control | 3 |
| CD4 Unfractionated | 37 |
| CD4 Mr<50 | 10 |
| CD4 Mr>50 | 41 |
| CD8 Unfractionated | 26 |
| CD8 Mr<50 | 9 |
| CD8 Mr>50 | 29 |

Again, both CD4 and CD8 supernatants suppressed HIV-1 replications. More inhibitory activity, however, was derived from the CD4 fraction. The supernatants did not affect the viability of CD4 cells.

Addition of the supernatant to CD4 cells in culture with IL-2 increased proliferation as data set forth in Table 2 and FIG. 3 demonstrates.

TABLE 2
FOLD INCREASE

| HIV + Fraction Dilution | Day 3 | Day 6 | Day 8 |
|---|---|---|---|
| Control | 3.2 | 13.6 | 21.6 |
| HIV | 3.4 | 10.4 | 15.6 |
| HIV + 1:160 | 5.3 | 13.6 | 20.4 |
| HIV + 1:40 | 5.5 | 13.6 | 26 |
| HIV + 1:10 | 5 | 13 | 21.2 |

HIV-1 Suppressive Activity is Mediated by a Factor(s) of 70 to 80 kDa

Supernatants from CD4 cells were separated into fractions of greater than 50 kDa and less than 50 kDa. Chemokines such as MIP-α (7.5 kDa), MIP-1β (7.8 kDa), RANTES (7.8 kDa), and IL-8 (8 kDa), are less than 50 kDa. CAF appears to be a small protein as it can pass through a 30 kDa cutoff filter. Mackewica, et al., “Effect of cytokines on HIV replication in CD4+ lymphocytes: lack of identify with the CD8+ cell antiviral factor”, Cell Immunol, 153:329-343, 1994. Cytokines such as IFN-α (19 kDa), IFN-β (18.5 kDa), and TGF-β (25 kDa) also are less than 50 kDa. Most active soluble members of the TNF family produced by activated lymphocytes exist as trimers greater than 50 kDa, including sFasL (70 to 80 kDa) and TNF-α (approximately 50 kDa). Tanaka, et al., “Expression of the functional soluble form of human Fas ligand in activated lymphocytes”, EMBO J, 14:1129-1135,1995 and Yoshimura, et al., “Molecular weight of tumor necrosis factor determined by gel permeation chromatography alone or in combination with low-angle laser light scattering”, Biochemistry International, 17:1157-63, 1988. Members of the TNF family also can exist as monomers in soluble form, including sFasL (27 kDa) and TNF-α (25 kDa). Virtually all of the soluble FasL detectable by ELISA was in the M, greater than 50 kDa fraction (500 pg/ml v. 110 pg/ml in the <50 kDa fraction), and all (200 pg/ml) of the MIP-1β was in the less than 50 kDa fraction. Moreover, the uninfected purified CD4 cells were cultured with HIV-1 and unfractionated supernatant, a greater than 50 kDa fraction inhibited IV-1 mRNA replication in CD4 cells as reported in Table 2 and FIG. 2.

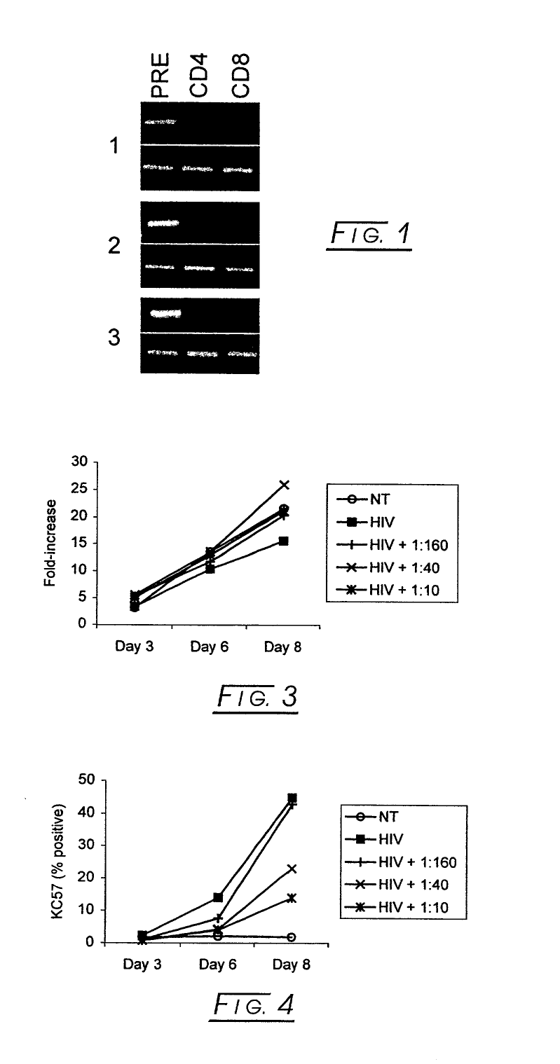

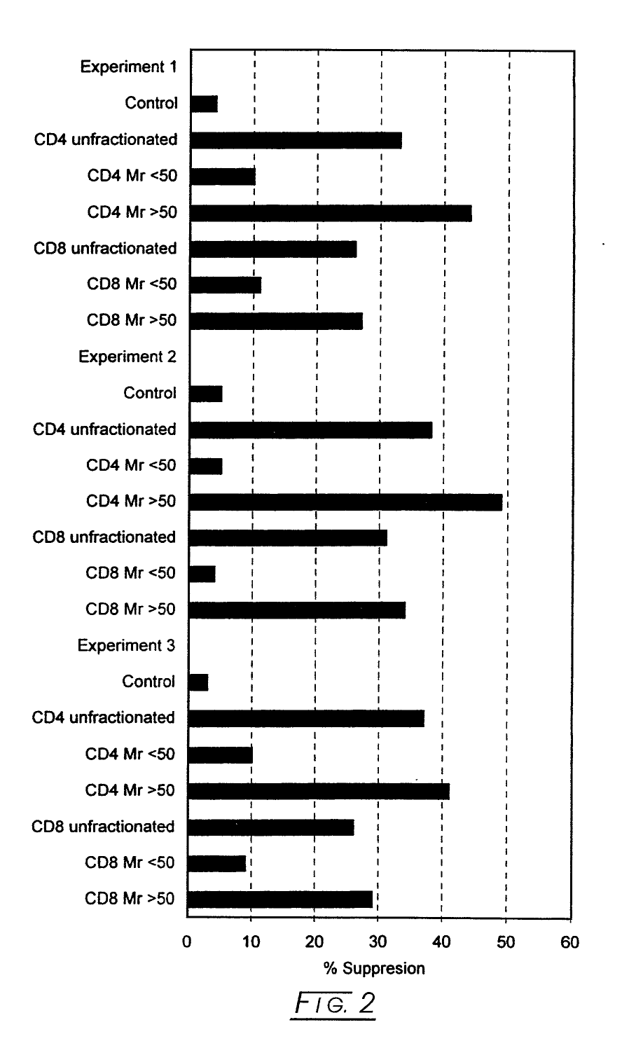

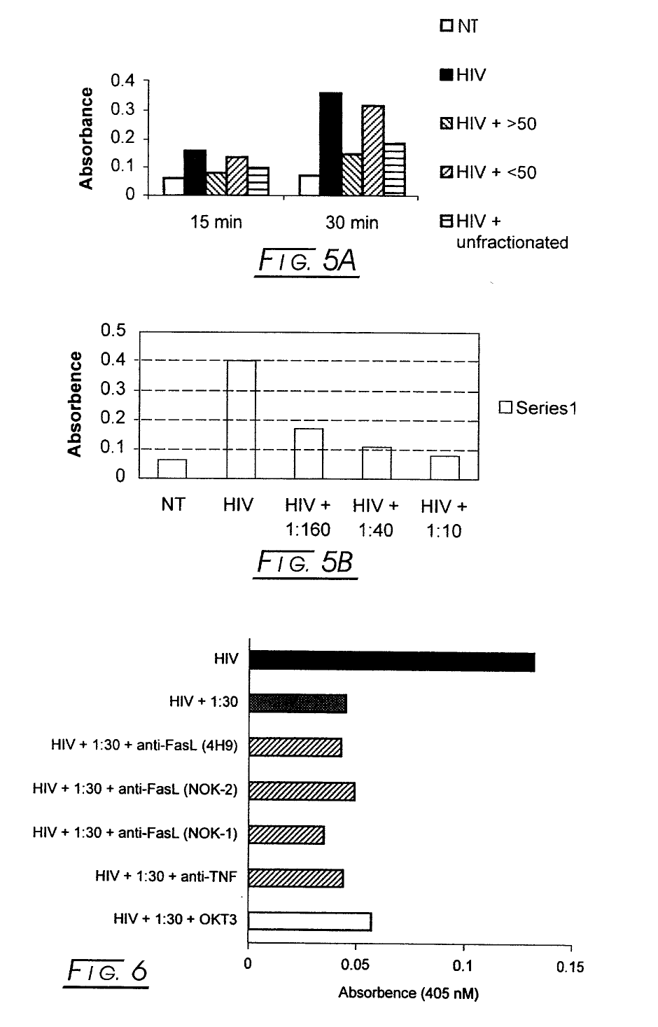

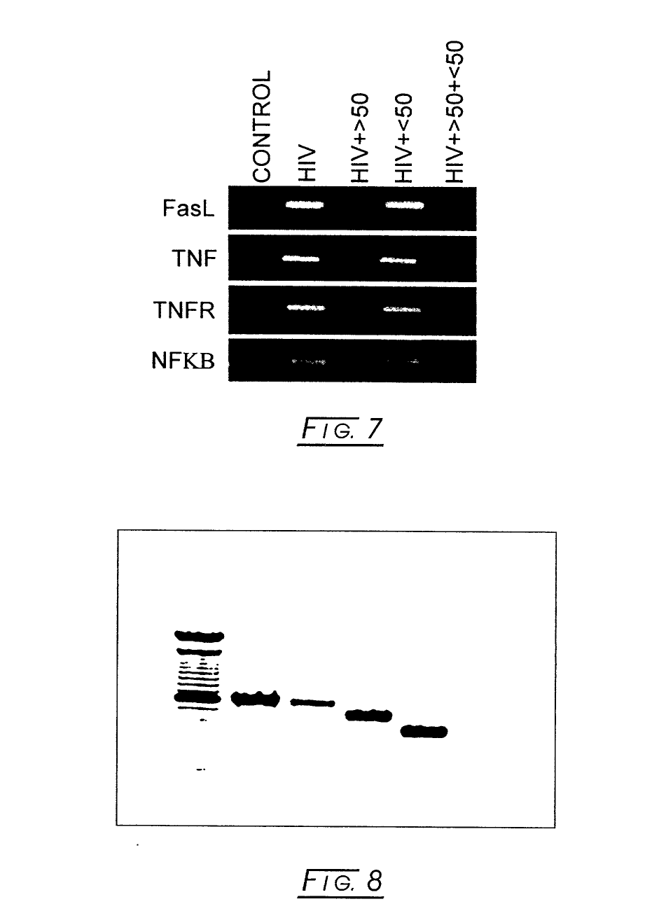

Supernatants were subjected to Superose 12 sizing, ConA and blue sepharose affinity, DEAE anion exchange, and Mono-P isoelectric columns. Activity was isolated to a fraction of 70 to 80 kDa. Flow cytometry with the KC57 antibody (which identifies the 55, 39, 33, and 24 kDa protein of the core antigens of HIV-1) was used to identify HIV-1 positive CD4+ cells and assess the activity of this purified fraction. CD4 cells were analyzed on days 3, 6 and 8 post infection. The number of HIV-1 positive CD4+ cells decreased in a dose dependent manner as the data in Table 3 demonstrates. This data also is plotted in FIGS. 4.

TABLE 3

| HIV + Fraction Dilution | KC57 (% POSITIVE) | | |
|---|---|---|---|
| Control | 1.8 | 2.1 | 1.8 |
| HIV | 2.3 | 14 | 44.6 |
| HIV + 1:160 | 1.1 | 7.6 | 42.7 |
| HIV + 1:40 | 0.8 | 4.2 | 22.9 |
| HIV + 1:10 | 0.8 | 4 | 13.8 |

The purified factor, Factor C, did not decrease the viability of the CD4 cells. In most experiments, increased proliferation was observed in HIV-1 infected CD4 cells cultured in IL-2 with the addition of the purified factor.

Soluble Factor(s) Produced by Activated-Expanded CD4 Cells Suppress LTR-driven

Transcription

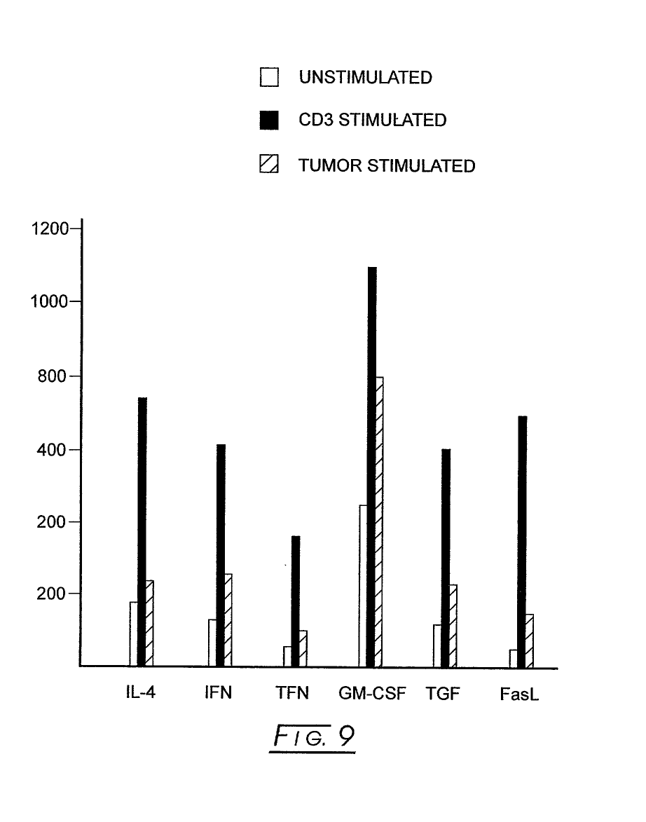

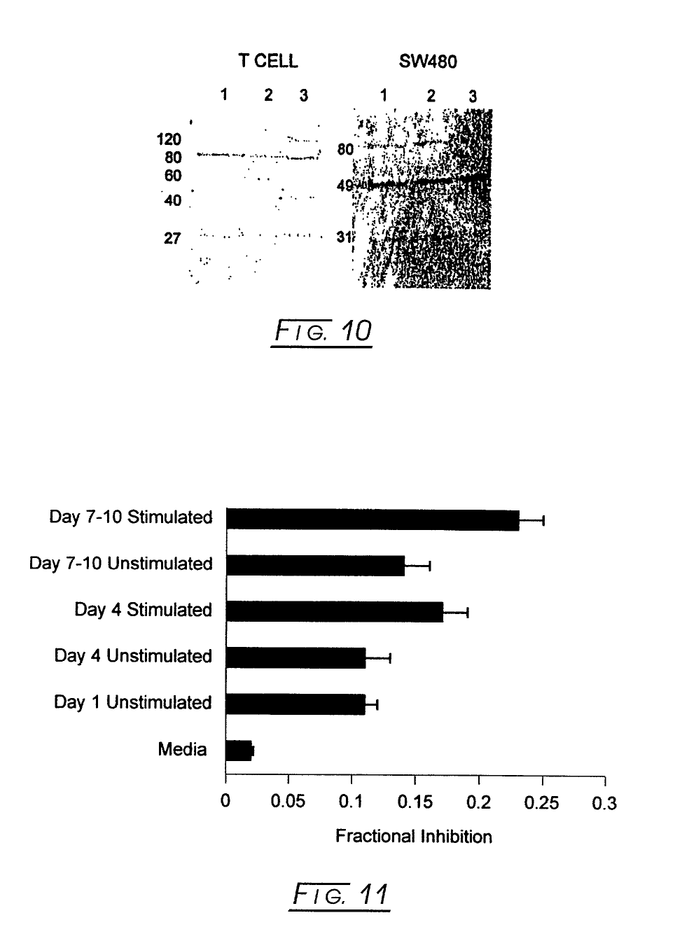

HeLa-Cd4-LTR-β-gal cells are HeLa cells infected with a retroviral vector expressing CD4 and with a truncated HIV-1 LTR-β-gal plasmid containing a hygromycin resistance gene. HIV-1 infection can be determined by infecting this cell line and staining for β-gal expression. The results of this experiment are set forth in Tables 4A and 4B below and in FIGS. 5A and 5B, respectively

TABLE 4A
TIME TEST-FIG. 5A

| HIV + FACTOR | % Absorbence--15 min | % Absorbence--30 min |
|---|---|---|
| Control | 0.06 | 0.07 |
| HIV | 0.157 | 0.362 |
| HIV + >50 kDa fraction | 0.079 | 0.146 |
| HIV + <50 kDa fraction | 0.135 | 0.318 |
| HIV +Unfractionated | 0.098 | 0.185 |

TABLE 4B
DILUTION TEST-FIG. 5B

| Dilution Test | % Absorbence |
|---|---|
| Control | 0.06 |
| HIV | 0.4 |
| HIV + >50 kDa fraction | 0.17 |
| HIV + <50 kDa fraction | 0.11 |
| HIV + Unfractionated | 0.08 |

The greater than 50 kDa fraction suppressed the ability of HIV-1 to enhance LTR-driven transcription in this model cell line after 15 minutes. The purified factor also directly inhibited LTR-driven transcription.

The Soluble Factor(s) Produced by Activated-Expanded CD4 Cells and That Suppresses HIV is not Mediated by TNF-α or sFasL

Factors greater than 50 kDa that have been reported to inhibit HIV-1 in CD4 cells include TNF-α and FasL. The effects of TNF-α and FasL were examined by evaluating the effects of recombinant formulations, alone and in combination, on LTR-driven transcription and by blocking studies. The data recorded is set forth in Table 5 and in FIG. 6.

TABLE 5

| HIV + FACTOR + ANTIBODY | ABSORBENCE (405 NM) |
|---|---|
| HIV + 1:30 + OKT3 | 0.057 |
| HIV + 1:30 + Anti-TNF | 0.044 |
| HIV + 1:30 + Anti-FasL (NOK-1) | 0.035 |
| HIV + 1:30 + Anti-FasL (NOK-2) | 0.049 |
| HIV + 1:30 + Anti-FasL (4H9) | 0.043 |
| HIV + 1:30 | 0.045 |
| HIV | 0.132 |

These data demonstrate that anti-FasL and anti-TNF-α antibodies could not decrease the suppressive effects of the purified factors. Such results are further proof that a new anti-transcription factor has been discovered.

Soluble Factor(s) Produced by Activated-Expanded CD4 Cells Block the Increase in NFKβ, Fas Ligand, and Tumor Necrosis Factor Induced by HIV-1

In addition, the factor blocked the increase in NF-Kβ, Fas ligand, and tumor necrosis factor expression in CD4 cells that is induced with HIV-1 infection. This data is presented in FIG. 7.

### Example 2

### Oncology Data

### Experimental

Cell Lines and Reagents

Human colorectal carcinoma cell lines LS174T and SW480 and T cell leukemia cell lines Jurkat were obtained from the American Type Culture Collection (ATCC, Rockville, Md.). Cells were cultured at 37° C. in 5% CO₂ in their maintenance media, which consisted of RPMI-1640 with 2 mM glutamine and 10% fetal bovine serum (FBS, Gibco BRL, Grand Island, N.Y.). Human recombinant TNF, human recombinant IFN-γ, anti-TNF antibody, anti-IFN-γ (R&D Systems Inc., Minneapolis, Minn.), sFasL (Oncogene, Cambridge, Mass.), anti-FasL antibody (NOK1, ParMinigen), and anti-FasR IgM antibody (Coulter Corporation, Miami, Fla.) were obtained the commercial sources indicated.

Lymphocyte Culture

Lymphocytes were separated from lymph nodes obtained by the procedure described by Triozzi, et al., “Adoptive immunotherapy using lymph node lymphocytes localized in vivo with radiolabeled monoclonal antibody”, J Natl Cancer Inst, 87:1180-1181 (1995). Lymph node cells were suspended at 10⁶/ml in expansion media, which consisted of modified AIM-V (Macrophage-SFM, Gibco BRL) with 10 μg/ml gentamicin to which 100 U/ml of human recombinant interleukin-1 (IL-2) (Proleukin, Cetus Oncology Corporation, Emeryville, Calif.) and 10 ng/ml anti-CD3 antibody (OKT3, Ortho Biotech, Raritan, N.J.) were added. Cells were cultured at 37° C. for 4 days and then resuspended at 0.25×10⁵/ml in expansion media containing 20 U/ml IL-2 for 3 days and at 0.5×10⁶/ml in expansion media containing 20 U/ml of IL-2 for 3 more days.

T-cell Stimulation

“Stimulated” supernatant consisted of supernatants collected after the day-10 lymphocytes had been recultured in vitro at 10⁶/ml in expansion media for an additional 24 hours in T75 plastic flasks (Becton Dickinson Labware, Franklin Lakes, N.J.) in which anti-CD3 mAb had been previously immobilized by culturing in Hank's Balanced Salt Solution for 18 hours. “Unstimulated” supernatant consisted of the supernatant collected at day −10 of lymphocyte expansion after centrifugation at 200 g. Both freshly collected supernatants and supernatants that had been frozen at −20° C. and then thawed were evaluated. Supernatants also were centrifuged at 100× g for 30 minutes in Millipore Ultrafree Biomax (Bedford, Mass.) filter devices with nominal M_{Γ} 50,000 limits. The concentrated supernatants were collected and then diluted.

Proliferation Assay

Unstimulated and stimulated supernatants, as well as the expansion media, were added at a range of volumes to SW480, LS174T, or Jurkat cells or cultured in their maintenance media in 24-well plates for 96 hours. MTS was added for the final 5 hours of culture as recommended by the manufacturer (CelTiter, 96 AQᵤₑₒᵤₛ Non-Radioactive Cell Proliferation Assay, Promega, Madison, Wis.), and the absorbence was read in a DigiScan reader (ASYS Hitech, Austria) at A₄₉₂ nm. Samples were evaluated in triplicate. Tumor cells cultured in maintenance media with no addition and the maintenance media alone were used as controls. Fractional inhibition was determined by the following formula:

$$\frac{( experimental absorbence - \\ absorbence of maintenance media alone )}{( absorbence of maintenance media with no additions - \\ absorbence of maintenance media alone ) .}$$

Flow Cytometry

Flow cytometry was used to assess FasR expression and cell cycle. Analysis of cell cycle was performed using propidium iodide on a Coulter EPICS Elite flow cytometer (Coulter Corporation) equipped with a 488 nm, 15 mW, air-cooled Argon laser. (see Darzynkiewicz, et al., Methods in Cell Biology: Flow Cytometry, 2^{nd} Edition, Part A, pp 32-36, 1995). Optical laser alignment calibration of the flow cytometer was performed using Coulter's DNA-Check EPICS alignment fluorosphere beads with coefficient of variations routinely less than 2%. PI fluorescent light emission was collected through a 610 nm, long-pass transmission filter. PI signal was measured in linear mode and extended analysis of DNA content was performed using the ModFit LT program (Verity Software House, Inc., Topsham, Me.). Data are presented as the percentage of cells in G1-G0, S and G2-M.

Enzyme-linked Immonoabsorbent Assay (ELISA)

Commercially available enzyme-linked immunoabsorbent assay (ELISA) kits were used to quantify TNF-α, FasL, IL-4, IFN-γ, TGF-γ, and GM-CSF (R&D Systems, Inc.). Assays were conducted in duplicate according to the recommendations of the manufacturer.

DNA Fragmentation

Detection of DNA fragmentation by “laddering” was performed using the Apoptosis ladder kit from Boehringer Mannheim. Briefly, 2×10⁶ cells were lysed in a cell lysis buffer and the nucleic acid released was bound to the surface of a glass filter in the presence of a chaotropic salt. After washing, the bound DNA was eluted in an elution buffer that was pre-warmed at 70° C. DNA was separated in a 1% agarose gel. After electrophoresis gels were stained with ethidium bromide and the DNA was visualized under UV light.

Immonoblot Analysis

SW480 cells exposed to fractions from lymphocyte culture supernatants were harvested and suspended in SDS buffer. Cell extracts were boiled for 10 min and chilled on ice. Total proteins from SW480 cells were separated on a 0.8% SDA-PAGE and electrophoretically transferred to a PVDF membrane. The membranes were incubated with appropriate polyclonal antibodies (anti-rabbit IgG bcl-2 or Bax) (Calbiochem) for 6 to 8 hours and washed with TTBS and incubated with secondary antibody conjugated with alkaline phosphatase. The signal then was detected with BCPIP/NBT (5-bromo-4-chloro-3-indolyl-phosphate/nitro blue teterazolium) color substrate in an alkaline phosphatase buffer (100 mM Tris-HCl (pH 9.5), 100 mM NaCl, 5 mM MgCl₂).

Reverse Transcription Polymerase Chain Reaction

The expression of FasL, Bcl-2, and Bax was determined by reverse transcription of total RNA followed by PCT analysis (RT-PCR). Approximately 10⁶ cells were lysed in Trizol reagent (Life Technologies) and RNA was isolated according to the manufacturer's instructions. cDNA was synthesized by extension with random primers with 200 units of Super script 11 reverse transcriptase (Life Technologies). The reaction mixture contained 1 μg of total RNA in a final volume of 20 μl. To determine the purity of RNA RT reactions also were performed on RNA samples without the enzyme and the samples were used in PCR reactions. The 2 μl cDNA was used in a 20 μl reaction volume containing all four dNTPs (10 μM), mM MgCl₂ and 2.5 units of Taq polymerase (Life Technologies) and each primer at 1 μM. The amplification cycles were 94° C. for 30 seconds, 60° C. for 2 minutes (×30). Primers used for amplification were FasL sense primer corresponding to nucleotides 110-131 (5′-TCC TTG ACA CCT CAG CCT CTA-3′), and antisense primer complimentary to nucleotides 713-693 (5′-CCT CAC TCC AGA AAG CAG GAC-3′). The amplified products from the PCR reaction were separated on 1 % agarose gel and visualized by ethidium bromide staining. To detect the levels of NF-Kβ, the amplification cycles were 94° C. for 30 sec. 60° C. for 30 sec. and 72° C. for 1 minute (×30). Primers used for amplification were NF-Kβ sense primer corresponding to nucleotides 1792-1812 (5′-CTT TCT GCT GCG GGT AGG TG-3′), and antisense primer complimentary to nucleotides 2707-2687 (5′-GCT TGT CTC GGG TTT CRG GA-3′). The amplified products from the PCR reaction were separated on 1% agarose gel and visualized by ethidium bromide staining.

N-α-benzyloxycarbonyl-L-lysine thiobenzyl esterase (BLT-esterase)

Granzyme A activity was assessed by BLT-esterase as described by Hammond, et al., “Double-negative T Cells from MRL-lpr/lpr Mice mediate cytolytic activity when triggered through adhesion molecules and constitutively express perforin gene”, J Exp Med, 178:2225 (1993). Briefly, 1×10⁶ Il-2 activated peripheral blood lymphocytes, i.e., lymphokine activated killer (LAK) cells, were lysed in RPMI (Gibco) containing 1% Triton X-100 (Sigma Chemicals, St. Louis, Mo.) and used as a positive control for Granzyme A. This lysate (20 μl) or supernatants from the anti-CD34IL-2 generated tumor-reactive lymphocytes were added to 96 well flat bottom microtiter plates in triplicate containing 180 μl of assay solution. Assay solution consisted of 2.2×10⁻⁴ M of 5,5′-dithio-bis(2-nitro)-benzoic acid (Sigma), 2.0×10⁻⁴ M of N α-CBZ-L-Thio Benzyl Ester (Sigma), and PBS. The plate then was incubated at room temperature overnight and read at a wavelength of 405 nm in an ELISA plate reader (Bio Tek Instruments).

### Results

Soluble Products

The 10-day activation and expansion regimen yielded a mixed population of CD4+ and CD8+ T cells. Virtually all of the cells express FasR by flow cytometry; however, FasL could not be detected by flow cytometry. The activated-expanded cells did express mRNA for FasL and other members of the TNF family, including TNF-β, and TRAIL. These results are evident in FIG. 8, which shows: GAPDH (lane 20, FasL (lane 3), TRAIL (lane 4), and TNF-α (lane 5) mRNA expression of the activated an expanded cells. Lane 1 is a 100 bp reference. The activated-expanded cells also expressed IFN-γ, IL-4, GM-CSF, and TGF-β (see Tanaka, et al., “Downregulation of Fas ligand by shedding”, Nature Med, 4:31-36, 1998; and Kim, et al., “Expansion of mucin-reactive lymph node lymphocyte subpopulations form patients with colorectal cancer”, Cancer Biother, 10:115-123, 1995). The levels of various factors that are observed in the supernatants of the activated-expanded cells at day 10, and with and without further stimulation with autologous tumor or anti-CD3 mAb, are presented in Table 6 and FIG. 9.

TABLE 6

| Factor | Unstimulated | | CD3 Stimulated | | Tumor Stimulated | |
|---|---|---|---|---|---|---|
| IL-4 | 175.1667 | 238 | 771 | 343 | 225 | 25 |
| IFN | 143.33333 | 162 | 604 | 298 | 250 | 25 |
| TNF | 65.16667 | 45 | 365 | 45 | 100 | 10 |
| GM-CSF | 450 | 200 | 1100 | 299 | 800 | 85 |
| TGF | 110 | 23 | 600 | 213 | 230 | 25 |
| FasL | 50 | 20 | 700 | 200 | 150 | 15 |

The production of sFasL was induced and existed as a species of Mr 80,000 and 27,000 (see FIG. 10). Assessment of FasL of the tumor-reactive T cells after cell lysis indicated additional species at 40,000, 60,000 and 120,000. The supernatant did not demonstrate granzyme activity, which can activate apoptotic pathways in addition to their lytic activities, at days 8-10. Granzyme activity, however, was present at day 4. This result is consistent with the results presented by Garcia-Sanz, et al., “Appearance of granule-associated molecules during activation of cytolytic T-lymphocyte precursors by defined stimuli”, Immunology, 64:129-134 (1998).

Effects on Cell Growth and FasR

Colorectal tumor cell lines were cultured with a range of concentrations of supernatants from autologous-tumor and anti-CD3 mAb stimulations. Morphologic changes typical of apoptosis including membrane bleeding and chromatin condensation were apparent as early as 24 hours after exposure to the supernatants. Supernatants collected from activation-expansion tumor-reactive T cells inhibited the growth of colorectal cancer cell lines.

The effect of unstimulated supernatants and anti-CD3 mAb-stimulated supernatants, collected at various time points in the activation-expansion regimen, and the expansion media supplemented with 20 U/ml IL-2 on the growth of LS174T cells when added at a 25% volume/volume (v/v) to LS174T cells in maintenance media is well seen. This data is set forth in Table 7and FIG. 11.

TABLE 7

| | Fractional Inhibition | |
|---|---|---|
| Media | 0.02 | 0.002 |
| Day 1 Unstimulated | 0.11 | 0.01 |
| Day 4 Unstimulated | 0.11 | 0.02 |
| Day 4 Stimulated | 0.17 | 0.02 |
| Day 7-10 Unstimulated | 0.14 | 0.02 |
| Day 7-10 Stimulated | 0.23 | 0.02 |

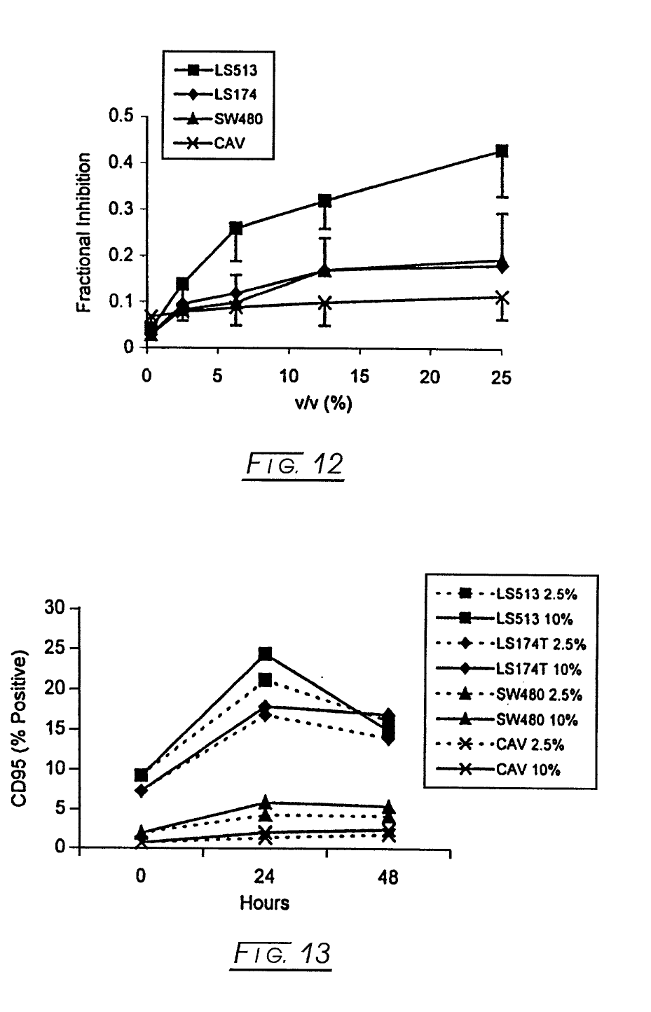

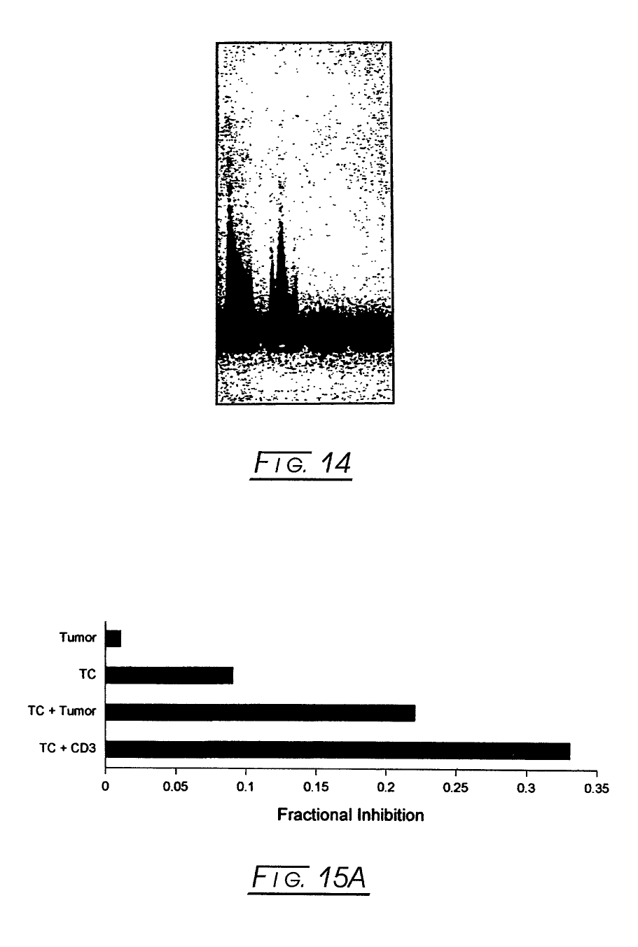

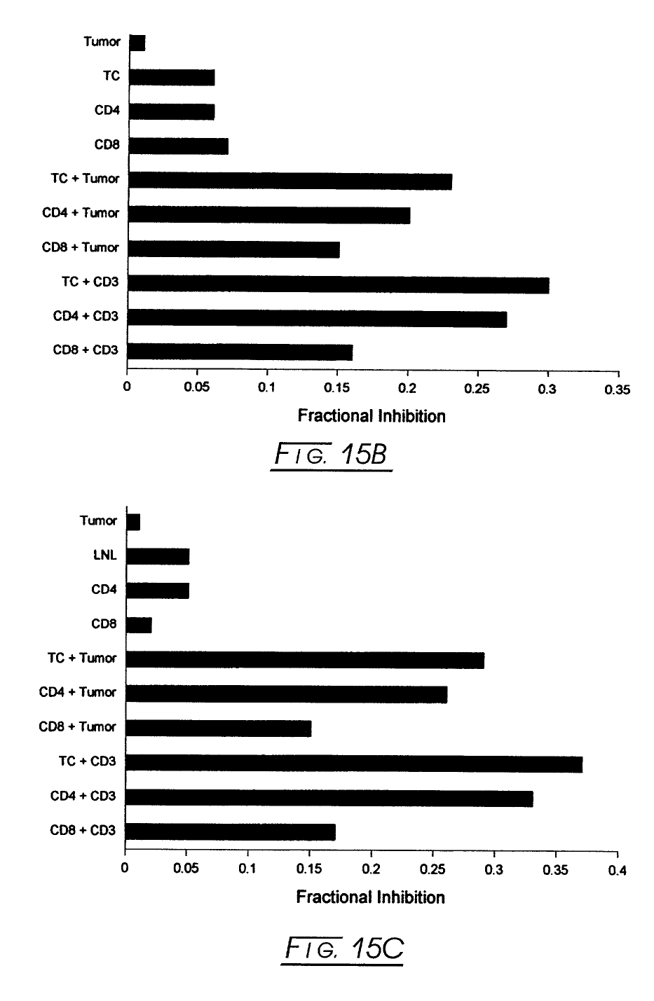

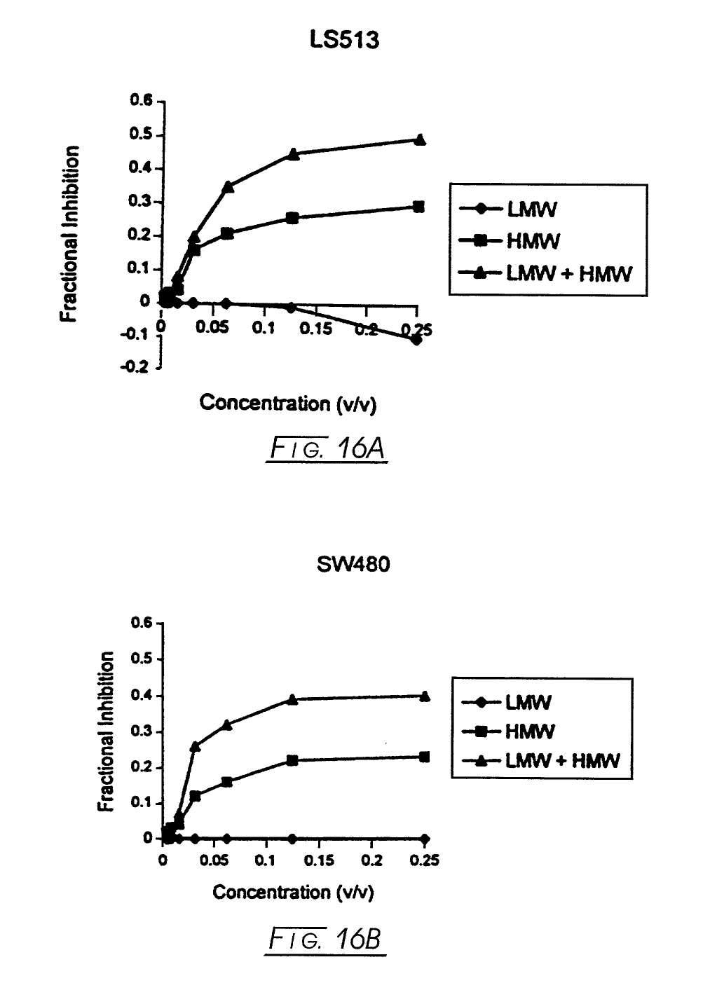

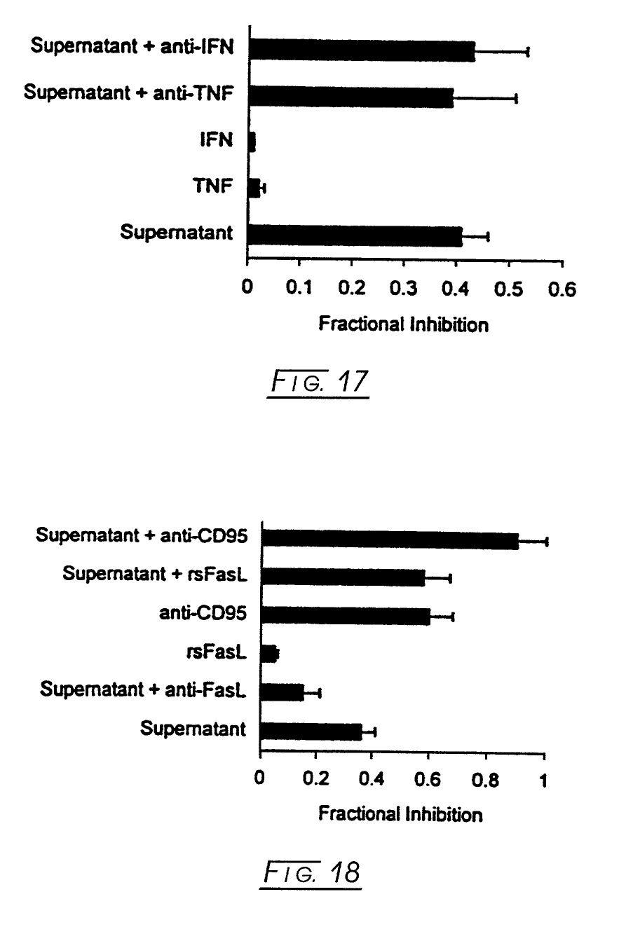

The effects of a range of concentrations of the anti-CD3 mAb stimulated supernatants on the grown of FasR (CD95) expression of SW480, LS174T, LS513, and CAV colorectal cancer cells were compared and the data is set forth below in Tables 8 and 9, and FIGS. 12 and 13, respectively.

TABLE 8

| | | Fractional | |
|---|---|---|---|
| Cell Type | v/v (%) | Inhibition | |
| LS513 | 0.25 | 0.043 | 0.03 |
| | 2.5 | 0.14 | 0.06 |
| | 6.25 | 0.26 | 0.1 |
| | 12.5 | 0.32 | 0.18 |
| | 25 | 0.431 | 0.24 |
| LS174 | 0.25 | 0.01 | 0.03 |
| | 2.5 | 0.06 | 0.097 |
| | 6.25 | 0.07 | 0.12 |
| | 12.5 | 0.06 | 0.19 |
| | 25 | 0.1 | 0.2 |
| SW480 | 0.25 | 0.01 | 0.03 |
| | 2.5 | 0.02 | 0.085 |
| | 6.25 | 0.02 | 0.1 |
| | 12.5 | 0.03 | 0.17 |
| | 25 | 0.08 | 0.195 |
| CAV | 0.25 | 0.01 | 0.07 |
| | 2.5 | 0.06 | 0.08 |
| | 6.25 | 0.06 | 0.09 |
| | 12.5 | 0.07 | 0.1 |
| | 25 | 0.1 | 0.12 |

TABLE 9

| Cell (v/v % Added) | Time 0 | Time 24 Hrs. | Time 48 Hrs. |
|---|---|---|---|
| LS513 (2.5%) | 9.25 | 21.2 | 16.2 |
| LS513 (10%) | 9.25 | 24.4 | 15 |
| LS174T (2.5%) | 7.34 | 16.9 | 14 |
| LS174T (10%) | 7.34 | 17.9 | 17 |
| SW480 (2.5%) | 1.93 | 4.28 | 4.17 |
| SW480 (10%) | 1.93 | 5.87 | 5.45 |
| CAV (2.5%) | 0.731 | 1.38 | 1.82 |
| CAV (10%) | 0.731 | 2.02 | 2.43 |

The expression of FasR by the tumor cells paralleled the sensitivity to the supernatants. Exposure to the supernatants induced the DNA fragmentation characteristic of apoptosis (see FIG. 14). The effects of the stimulated supernatant on the cell cycle indicated that the primary effect is an increase in cells in G0-G1.

T cells were separated into CD4 and CD8 fractions using immunobead techniques prior to re-stimulation with anti-CD3 or with autologous tumor. These results are displayed in Tables 10A-C and FIGS. 15A-C, wherein antiproliferative effects of supernatants derived from autologous tumor, unseparated activated-expanded T cell populations derived from lymph nodes (LNL) and CD4 and CD8 cells separated from this population after activation-expansion is displayed. Supernatants were collected from LNL, CD43, and CD8 populations after stimulation from anti-CD3 mAb (CD3) or with autologous tumor (Tumor). Three different activation-expansion-autologous systems (labeled A, B, and C) were evaluated.

| | |
|---|---|
| Cell Population | Fractional Inhibition |

TABLE 10A

| | |
|---|---|
| CD8 + CD3 | 0.16 |
| CD4 + CD3 | 0.27 |
| TC + CD3 | 0.3 |
| CD8 + Tumor | 0.15 |
| CD4 + Tumor | 0.2 |
| TC + Tumor | 0.23 |
| CD8 | 0.07 |
| CD4 | 0.06 |
| TC | 0.06 |
| No Treatment | 0.01 |

TABLE 10B

| | |
|---|---|
| CD8 + CD3 | 0.17 |
| CD4 + CD3 | 0.33 |
| TC + CD3 | 0.37 |
| CD8 + Tumor | 0.15 |
| CD4 + Tumor | 0.26 |
| TC + Tumor | 0.29 |
| CD8 | 0.02 |
| CD4 | 0.05 |
| LNL | 0.05 |
| Tumor | 0.01 |
| No Treatment | 0.01 |

TABLE 10C

| | |
|---|---|
| TC + CD3 | 0.33 |
| TC + Tumor | 0.22 |
| TC | 0.09 |
| Tumor | 0.01 |

Both the CD4 and CD8 fractions produced soluble factors that inhibited tumor cells growth after simulation with anti-CD3 mAb or autologous tumor. Most (>80%) of in inhibitory effect, however, was derived from the CD4 fraction. Two-color flow cytometry with anti-CD4, and anti-CD8, and propidium iodine, indicated that the cells proliferating to autologous tumor were primarily CD4+ cells.

Supernatants were separated into fractions with Mr greater than 50,000 and Mr less than 50,000. Most active, soluble members of the TNF family produced by activated lymphocytes exist as timers of M_{Γ} greater than 50,000, including sFasL (70,000 to 80,000) and soluble TNF-α (approximately 50,000). See Tanaka, et al., “Expression of the functional soluble form of human Fas ligand in activated lymphocytes”, EMBO J, 14:1129-1135, 1995; and Yoshimura, et al., “Molecular weight of tumor necrosis factor determined by gel permeation chromatography alone or in combination with low-angle laser light scattering”, Biochemistry International, 17:1157-63, 1988. Members of the TNF family also can exist as monomers in soluble form, including sFasL (Mr 27,000) and TNF-α (25,000). Cytokines such as IFN-γ (8,000), IL-4 (14,000), GM-CSF (26,000), oncostatin M (26,000), and TGF-β (25,000), all have M_{Γ} of less than 50,000. Content of lytic granules include perforin (Mr 65,000), granzyme A (60,000) granzyme B (29,000), and granzyme C (27,000). Virtually all the soluble FasL detectable by ELISA was in the Mr greater than 50,000 fraction (500 pg/ml v. 110 pg/mI in the less than 50,000 fraction), and all (420 pg/ml) of the IFN-γ was in the less than 50,000 fraction. The antiproliferative activity and the capacity to induce FasR (CD95) were present in the Mr greater than 50,000 fraction, as the data set forth below in Table 11, and FIGS. 16A and 16B indicate.

TABLE 11

| Concentration | Mᵣ>50 | Mᵣ<50 | Supernatant |
|---|---|---|---|
| LS 513 Cells | | | |
| 0.3906 | 0.03 | 0 | 0.02 |
| 0.7813 | 0.03 | 0 | 0.02 |
| 1.5625 | 0.04 | 0 | 0.08 |
| 3.125 | 0.16 | 0 | 0.2 |
| 6.25 | 0.21 | 0 | 0.35 |
| 12.5 | 0.26 | −0.01 | 0.45 |
| 24 | 0.3 | −0.1 | 0.5 |
| SW480 Cells | | | |
| 0.3906 | 0.02 | 0 | 0.01 |
| 0.7813 | 0.03 | 0 | 0.02 |
| 1.5625 | 0.04 | 0 | 0.07 |
| 3.125 | 0.12 | 0 | 0.26 |
| 6.25 | 0.16 | 0 | 0.32 |
| 12.5 | 0.22 | 0 | 0.39 |
| 24 | 0.23 | 0 | 0.4 |

The less than 50,000 fraction actually stimulated the growth of LS174T cells. Antiproliferative activity of unfractionated supernatant, however, was greater than the Mr greater than 50,000 fraction alone.

Role of sFasL

The effects of FasL and other known immunologic mediators of apoptosis were examined by evaluating the effects of recombinant formulations, alone and in combination, on the growth of SW480 cells, and by blocking studies, as the data is set forth below in Tables 12 and 13, and in FIGS. 17 and 18, respectively.

TABLE 12

| | Fractional | Fractional |
|---|---|---|
| Additive | Inhibition - Run 1 | Inhibition - Run 2 |
| Supernatant | 0.36 | 0.41 |
| Supernatant + anti-FasL | 0.15 | 0.21 |
| rsFasL | 0.05 | 0.06 |
| Anti-FasR | 0.6 | 0.68 |
| Supernatant + rsFasL | 0.58 | 0.67 |
| Supernatant + anti-FasR | 0.9 | 1.0 |

TABLE 13

| | Fractional | Fractional |
|---|---|---|
| Additive | Inhibition - Run 1 | Inhibition - Run 2 |
| Supernatant | 0.41 | 0.46 |
| TNF | 0.02 | 0.03 |
| IFN | 0.01 | 0.011 |
| Supernatant + anti-TNF | 0.39 | 0.51 |
| Supernatant + anti-IFN | 0.43 | 0.53 |

Human recombinant sFasL, M_{Γ} of approximately 40,000, had insignificant effects on cell growth (see FIG. 17). Human recombinant TNF and IFN-γ also had insignificant effects on the growth of SW480 cells. In contrast, SW480 cells were sensitive to a murine IgM anti-FasR antibody. Treatment with anti-FasR antibody of IgM subclass appears to mimic mFasL and does induce apoptosis in Fas-sensitive cells. This apoptosis inducing ability is probably due to its ability to cross-link with FasR for efficient transmission of a cell death signal. See Bazzoni, et al., “Seminars in Medicine of the Beth Israel Hospital, Boston: The Tumor Necrosis Factor Ligand and Receptor Families”, New Engl J Med, 334:1717-1725, 1996. Anti-FasL antibody NOK1 could decrease the growth-inhibitory effects of supernatants (see FIGS. 14 and 15). Anti-IFN-γ and anti-TNF blocking antibody had no effect (see FIG. 17). The capacities of the recombinant sFasL and anti-FasR IgM to inhibit growth were enhanced by the addition of anti-CD3-IL-2 activated-expanded T-cell supernatant.

Effect on Tumor FasL, Bcl-2 and Bax

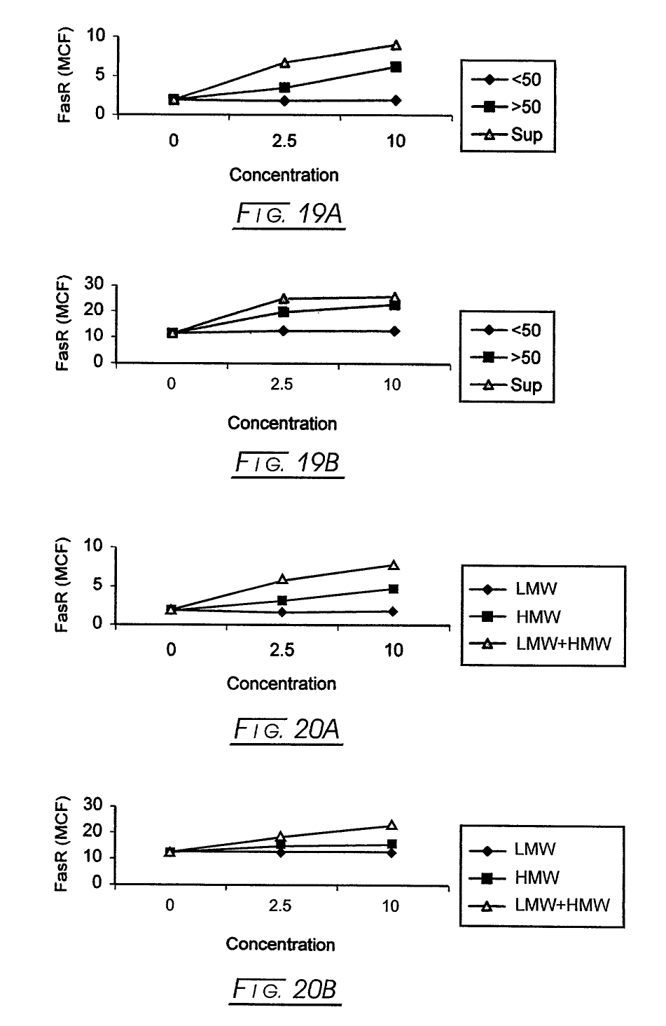

As had previously been reported, SW480 cells express FasL mRNA. See Tanaka, et al., “Fas ligand in human serum”, Nature Med, 2:317-322, 1996; and Shiraki, et al., “Expression of Fas ligand in liver metastases of human colonic adenocarcinomas”, Proc Natl Acad Sci USA, 94:6420-6425, 1997. FasL mRNA expression was not substantially modulated by the stimulated supernatant (or fraction), nor was FasL protein expression. These data are set forth below in Table 14, and FIGS. 19A and 19B, and 20A and 20B, respectively.

TABLE 14

| Concentration (%) | >50 Fraction | <50 Fraction | Supernatant |
|---|---|---|---|
| SW480 Cells @ 24 hours | | | |
| 0 | 1.87 | 1.87 | 1.87 |
| 2.5 | 1.74 | 3.44 | 6.69 |
| 10 | 1.83 | 6.14 | 8.97 |
| SW480 Cells @ 48 hours | | | |
| 0 | 1.87 | 1.87 | 1.87 |
| 2.5 | 1.68 | 3.2 | 6.14 |
| 10 | 1.78 | 5.16 | 7.92 |
| LS174 Cells @ 24 hours | | | |
| 0 | 11.5 | 11.5 | 11.5 |
| 2.5 | 12.6 | 19.8 | 25.1 |
| 10 | 13 | 23.1 | 26.1 |
| LS174 Cells @ 48 hours | | | |
| 0 | 11.5 | 11.5 | 11.5 |
| 2.5 | 11.5 | 15.4 | 18.5 |
| 10 | 11.5 | 15.6 | 21.8 |

FasL was not detectable in the supernatants of SW480 cells either by ELISA or immunoblotting. FasL could be detected in the cell lysates of SW480 cells and existed primarily as a secies of M_{Γ}0 of 49,000 (see FIG. 9). Supernatants collected from SW480 cells before exposure to T cell products did not inhibit the growth of FasL-sensitive Jurkat cells. Immune effectors elicit apoptosis by a variety of mechanisms. See Kreuser, et al., “Biochemical modulation of cytotoxic drugs by cytokines: molecular mechanisms in experimental oncology”, Recent Results Cancer Res, 139:371-82, 1995. Factors such as NF-KB, Bcl-2, and Bax may play roles. See May, “Control of apoptosis by cytokines”, Advances in Pharmacology, 41:219-246, 1998. NF-KB, a transcription factor, is induced in response to a variety of cytokines and blocks apoptosis. Bcl-2 is activated by chromosomal translocation and demonstrates a profound capacity to block apoptosis, probably by acting on downstream initiators, such as p53. Bax is a member of the Bcl-2 family and it antagonizes Bcl-2 and promotes apoptosis. The soluble factors had antiproliferative activity versus tumors with mutated p53, namely SW480, as well as non-mutated p53, namely LS174R. The M_{Γ} greater than 50,000 fraction of the stimulated supernatant downregulated Bcl-2 expression in SW480 cells, but did not modulate NF-KB mRNA or Bax protein expression (See FIG. 21). The M_{Γ} greater 50,000 fraction did enhance NF-KB expression (at 24 hours), but not modulate Bcl-2 or Bax.

### Example 3

### Further Oncology Data

### Experimental

Cell Lines and Reagents

Anti-CD3 mAb, recombinant IL-2, RPMI-1640 medium supplemented with 10% fetal bovine serum, and a serum-free medium, Macrophage SFM, containing antibiotics, as described above, were used. The following chemotherapeutics were evaluated:

Irinotecan HCl—Pharmacia and Upjohn Company, Bridgewater, N.J.

Topotecan—SmithKline Beecham, Philadelphia, Pa.

Human anti-TNF Ab, anti-IFN-γ, and anti-FasL Ab, as described above, also were used. Human colorectal carcinoma cell lines LS513 and SW480, as described above, were cultured at 37° C. in 5% CO₂ in their maintenance media, which consisted of RPMI-1640 with 2 mM glutamine and 10% fetal bovine serum.

Lymphocyte Culture and Supernatants

Lymphocytes were separated from lymph nodes obtained by the procedure described by Triozzi, et al., “Adoptive immunotherapy using lymph node lymphocytes localized in vivo with radiolabeled monoclonal antibody”, J Natl Cancer Inst, 87:1180-1181 (1995). Lymph node cells were suspended at 10⁶/ml in expansion media, which consisted of modified AIM-V (Macrophage-SFM, Gibco BRL) with 10 μg/ml gentamicin to which 100 U/ml of human recombinant interleukin-1 (IL-2) (Proleukin, Cetus Oncology Corporation, Emeryville, Calif.) and 10 ng/ml anti-CD3 antibody (OKT3, Ortho Biotech, Raritan, N.J.) were added. Cells were cultured at 37° C. for 4 days and then resuspended at 0.25×10⁶/ml in expansion media containing 20 U/ml IL-2 for 3 days and at 0.5×10⁶/ml in expansion media containing 20 U/ml of IL-2 for 3 more days. Day 10 cells were harvested by centrifugation (250× g, room temperature, 6 min) in 50 ml tubes. Two different formulations of the soluble products of the expanded TRL were evaluated: “Unstimulated” supernatant consisted on the supernatant collected at day-10 of the TRL expansion after centrifugation at 200× g; “Stimulated” supernatant consisted of supernatants collected after the day-10 TRL had been re-cultured in vitro at 10⁶/ml in expansion media for an additional 24 hours in T75 plastic flasks in which OKT3 had been previously immobilized by culturing in Hank's Balanced Salt Solution for 18 hours.

Proliferation Assay

Unstimulated and stimulated supernatants, as well as the expansion media, were added at a range of volumes to colorectal cancer cells or cultured in their maintenance media in 24-well plates for 96 hours. MTS was added for the final 5 hours of culture as recommended by the manufacturer (CelTiter, 96 AQᵤₑₒᵤₛ Non-Radioactive Cell Proliferation Assay, Promega, Madison, Wis.), and the absorbence was read in a DigiScan reader (ASYS Hitech, Austria) at A₄₉₂ nm. Samples were evaluated in triplicate. Tumor cells cultured in maintenance media with no addition and the maintenance media alone were used as controls. Fractional inhibition was determined by the following formula:

$$\frac{( experimental absorbence - \\ absorbence of maintenance media alone )}{( absorbence of maintenance media with no additions - \\ absorbence of maintenance media alone ) .}$$

Caspase Activity

Caspase-3 and caspase-8 activities were determined using a calorimetric assay kit (R&D Systems, Inc., Minneapolis, Minn.). Assays were conducted according to the instructions of the manufacturer.

Enzyme-linked Immonoabsorbent Assay (ELISA)

Commercially available enzyme-linked immunoabsorbent assay (ELISA) kits were used to quantify TNF-α, FasL, IL-1, IFNα, IFN-γ, and TGF-α, (IFN-α, BioSource International, Camarillo, Calif.; all others, R&D Systems, Inc.). Assays were conducted in duplicate according to the recommendations of the manufacturer.

Preparation of Cell-free Extracts

Media was removed and cells washed twice with TD (1:1 M NaCI, 41 mM KCl, 200 mM Tris pH 7.5, and 5 mM NaHPO₄). Cells were scraped into 1 ml TD and centrifuged for 3 minutes at 800× g, after which they were washed twice in TEM (10 mM Tris-HCl pH 7.5, 4 mM MgCl₂, and 1 nM EDTA). After allowing the cells to swell on ice for 10 minutes, the cells were homogenized using a Dounce homogenizer. Nuclei were pelleted by centrifugation at 1200× g for 5 minutes. Supernatant was discarded. The pellet was washed twice in TEM and resuspended in 50% TNEP (1×: 10 mM Tris-HCl pH 7.5, 150 mM NaCl, 1 mM EDTA, and 0.5 mM PMSF) and 50% 1 M NaCl, then chilled on ice for at least 10 minutes. Extracts then were centrifuged for 10 minutes in a microfuge and the supernatant removed to a clean tube and stored at −20° C. Total protein concentration was measured using a standard protein assay (Biorad) at A₅₉₅ on a Spectronic 1001 spectrophotometer (Milton Roy Co.).

Western Blotting

The amount of endogenous topoisomerase I protein was measured by Western blots. The amount of extracts from a cell line loaded onto a gel was normalized by total protein concentration. Extracts were run on an 8% SDA Page gel at 200 v. Gels were transferred to nitrocellulose membrane (Hybond) at 100 v for 1 hour at 4° C. Membranes were rinsed 3 times in TBST (20 mM Tris pH 7.5, 137 mM NaCl, and 0.1% Tween-20) for 1 minute each. They then were blotted in 5% dry milk in TBST for 3 hours and rinsed 3 times in TBST for 10 minutes. Membrane was incubated overnight in rabbit anti-human topoisomerase I antibody (TopoGEN) that was diluted 1:1000 in TBST, then washed 3 times in TBST. The secondary antibody used was 1¹²⁵-protein A (1 μC/ml). Results were viewed using autoradiography.

Statistical Analysis

The combined effects of the drugs and supernatants were analyzed by the median effect method of Chou and Talalay using CalcuSyn software (Biosoft, Ferguson, Mo.). In brief, when two agents are administered at a fixed ratio, a combination index (Cl) is calculated depending on whether the drugs are assumed to be mutually non-exclusive or mutually exclusive in their action (see Chou, et al., “Quantitative analysis of dose-effect relationships: the combined effects of multiple drugs or enzyme inhibitors”, Adv Enzyme Regul, 22:27-55, 1984). According to this method, “synergy” is indicated by a Cl of less than 1, “addition” by a Cl equal to 1, and “antagonism” by a Cl greater than 1. A Cl of less than 0.3 is considered to represent “strong synergism”, and a Cl greater than 3.3 is considered to represent “strong antagonism”.

### Results

Soluble Products

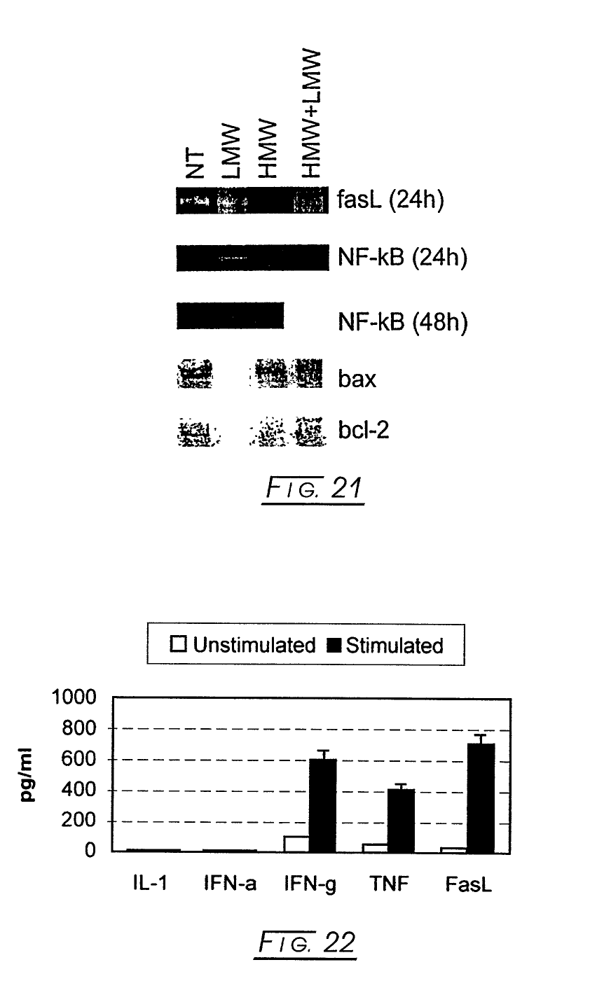

TRL were activated and expanded with anti-CD3 mAb and IL-2 as described by Kim, et al., “Expansion of mucin-reactive lymph node lymphocyte subpopulations form patients with colorectal cancer”, Cancer Biother, 10:115-123,1995). The cells that result from the culture regimen express mRNA for FasL and other members of the TNF family, including TNF-β and TRIAL, as well as IFN-γ, IL-4, granulocyte-macrophage colony stimulating factor (GM-CSF), and transforming growth factor-β, (TGF-β). They have been shown to secrete TNF-α, sFasL, IFN-γ, IL-4, GN-CSF, and TGF-β in response to tumor (Triozzi, et al., “Induction of Fas-mediated apoptosis by the soluble factors secreted by tumor-reactive T-cells”, submitted); GM-CSF and TGF-β, have been shown to promote the growth of several tumors. (Chou, et al., “Quantitative analysis of dose-effect relationships: the combined effects of multiple drugs or enzyme inhibitors”, Adv Enzyme Regul, 22:27-55, 1984; and Berdel, et al., “Effects of hematopoietic growth factors on malignant nonhematopoietic cells”, Seminars in Oncology, 19 (Suppl 4):41-45, 1992). The TRL generated do not produce significant IL-1α or IFN-α. Table 15 and FIG. 22 display the quantities of IL-1α, IFN-α, and TNF-α, cytokines reported in the art to modulate camptothecin activity, as well as the quantity of sFasL and IFN-γ present, before and after re-stimulation with anti-CD3 mAb.

TABLE 15

| | | |
|---|---|---|
| Cytokine | Unstimulated | Stimulated |

| | | | | |
|---|---|---|---|---|
| IL-1 | 11 | 1 | 11 | 1 |
| IFN-α | 11 | 1 | 11.5 | 1 |
| IFN-γ | 103 | 5.1 | 600 | 62 |
| TNF | 52 | 2.5 | 405 | 41 |
| FasL | 31 | 2 | 700 | 69 |

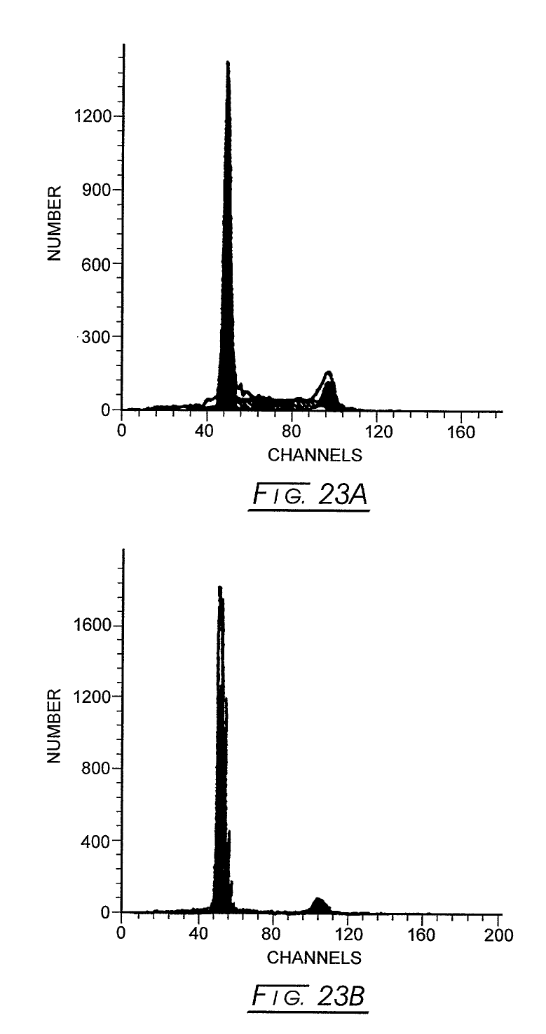

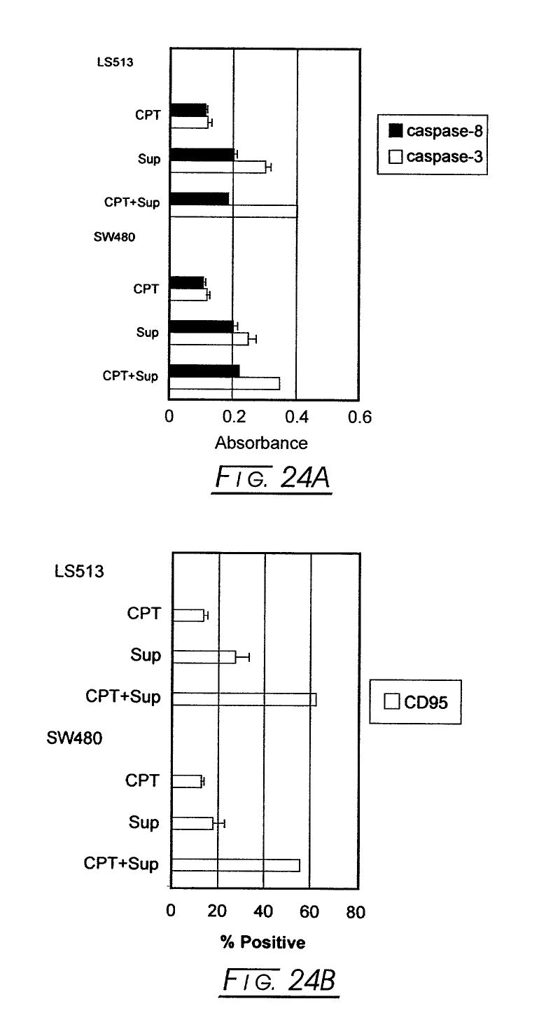

Although potentially growth stimulatory and growth inhibitory cytokines are present, the net effect of the TRL supernatants is to induce apoptosis of tumor targets and accumulation of cells in G10G0. This is demonstrated in FIG. 23. FasR expression is increased as is the activity of caspase-3, a “downstream” executioner caspase in the apoptotic pathway, and capase-8, a more proximal caspase that is triggered by FasL and other members of the TNF family. (Boldin, et al., “Involvement of MACH, a novel MORT1/FADD-interacting protease, in Fas/APO-1 and TNF receptor-induced cell death”, Cell, 85:803, 1006). This is demonstrated by the data displayed in Table 16 and FIG. 24.

TABLE 16

| | CD95 | | Caspase-3 | | Caspase-8 | |
|---|---|---|---|---|---|---|
| SW480 Cells | | | | | | |
| CPT + Sup | 56 | 5 | 0.35 | 0.3 | 0.22 | 0.02 |
| Sup | 18 | 1 | 0.25 | 0.06 | 0.2 | 0.005 |
| CPT | 13 | 1 | 0.12 | 0.005 | 0.11 | 0.005 |
| LS513 Cells | | | | | | |
| CPT + Sup | 62 | 6 | 0.4 | 0.02 | 0.18 | 0.01 |
| Sup | 27 | 2 | 0.3 | 0.007 | 0.2 | 0.005 |
| CPT | 13 | 1 | 0.12 | 0.003 | 0.11 | 0.0025 |

Combined Effects on Tumor Cell Growth

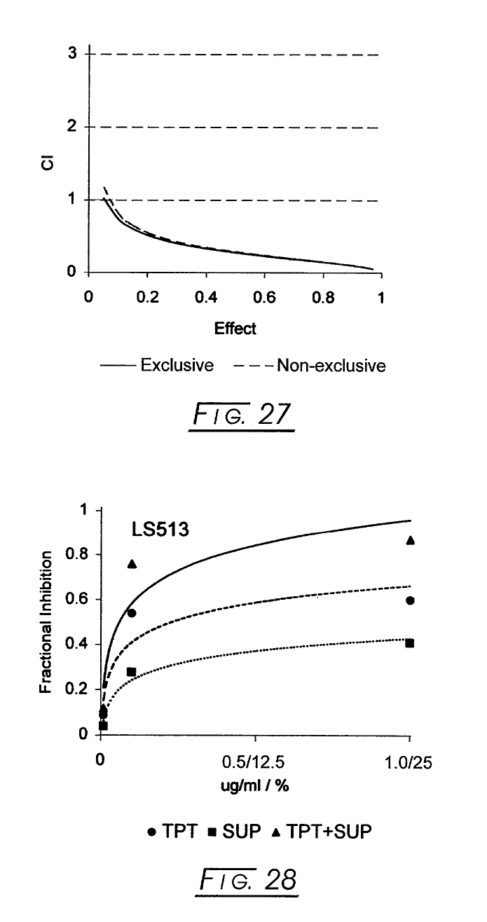

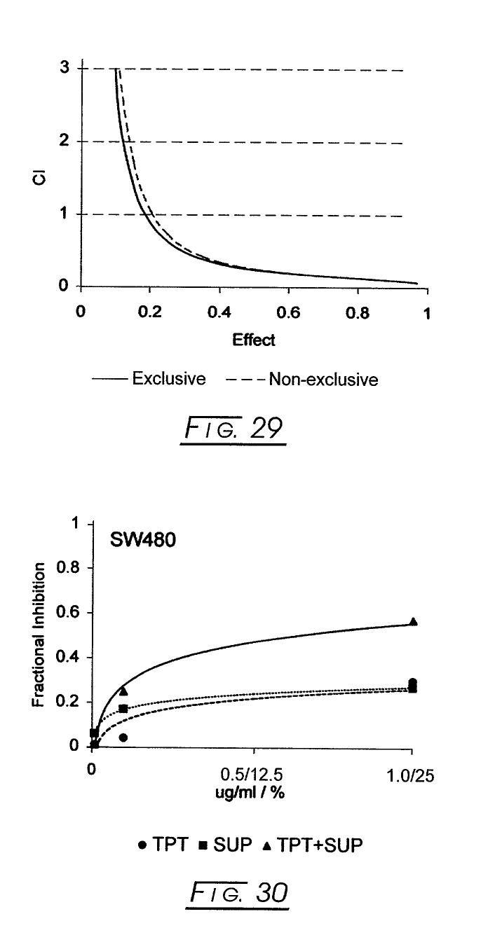

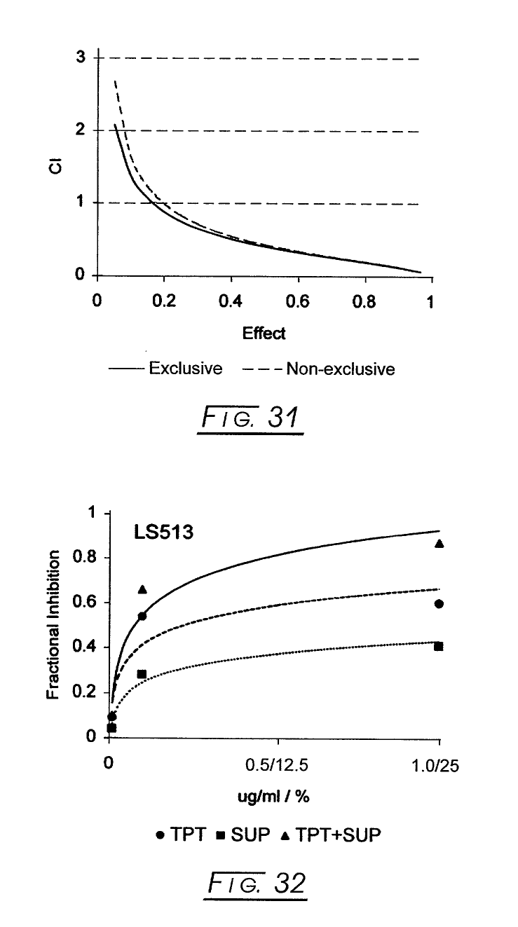

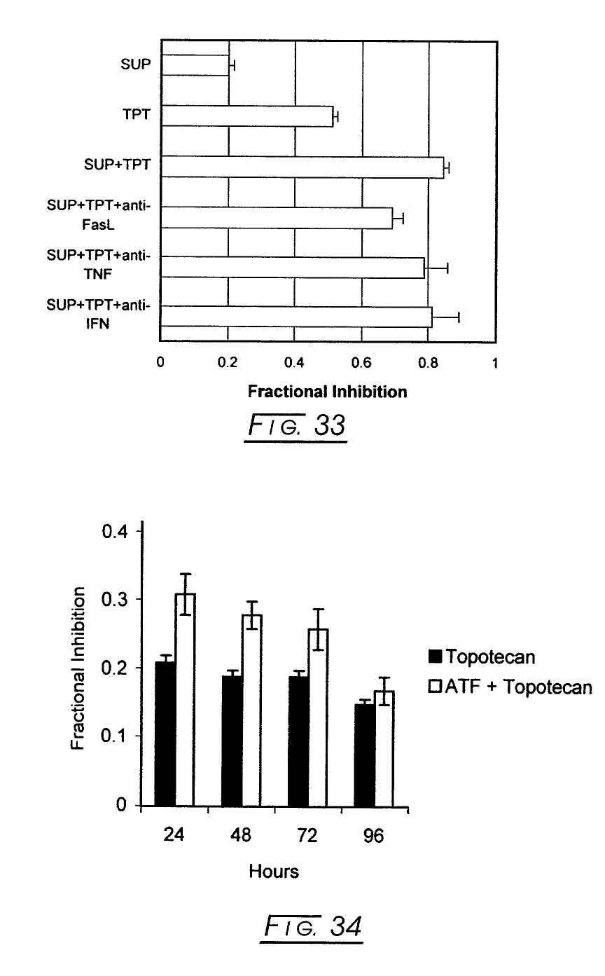

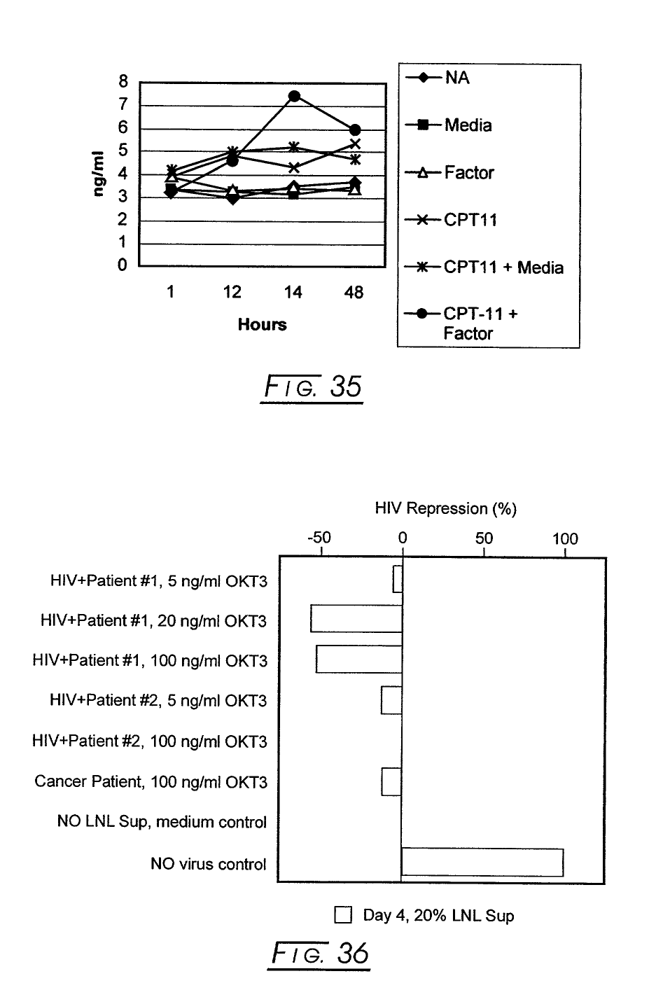

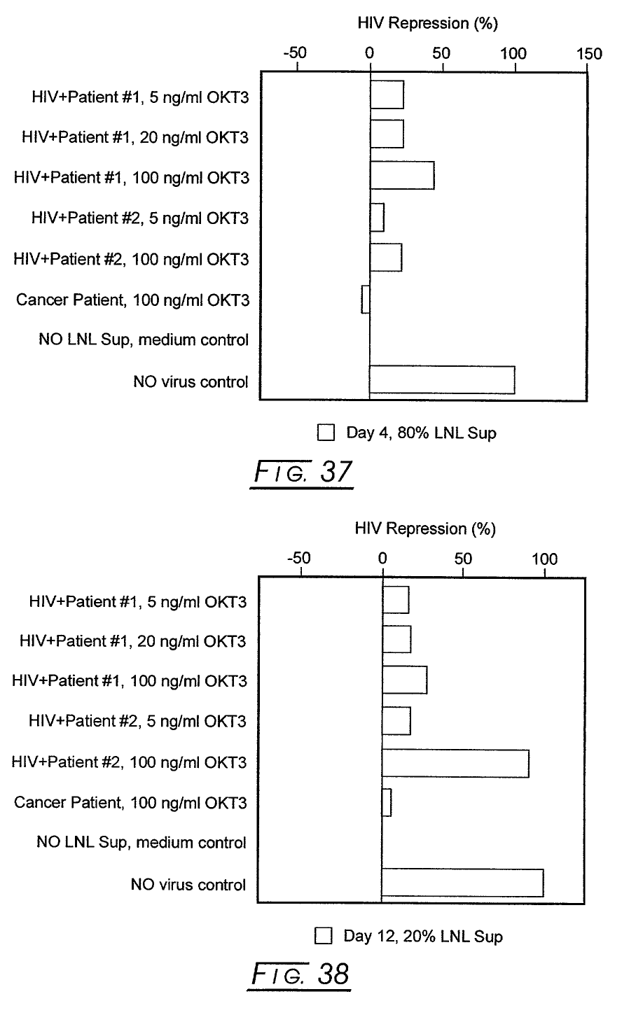

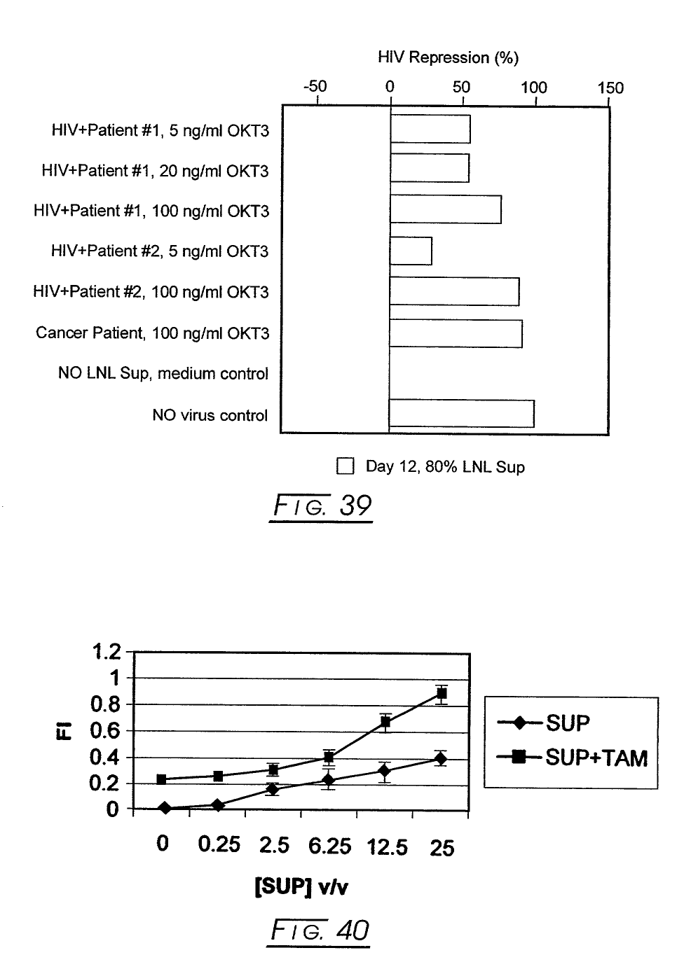

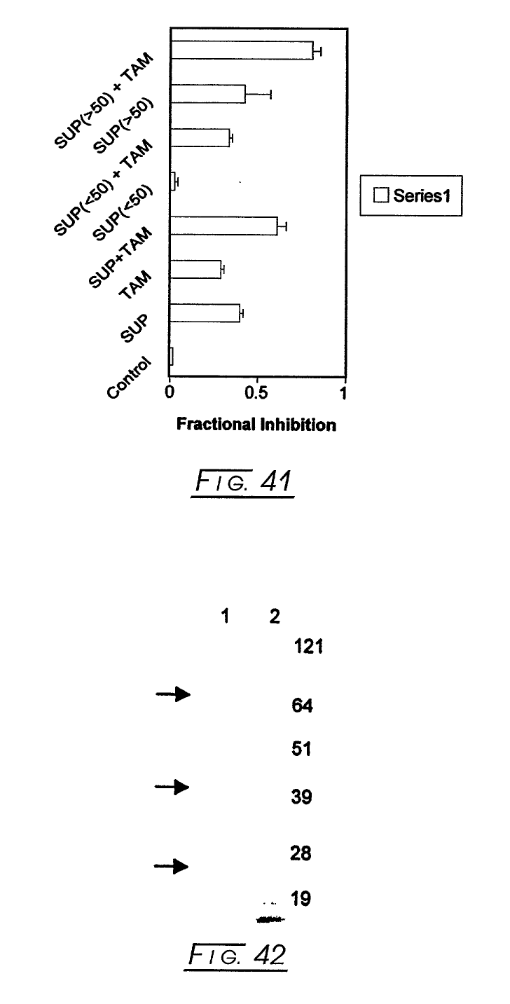

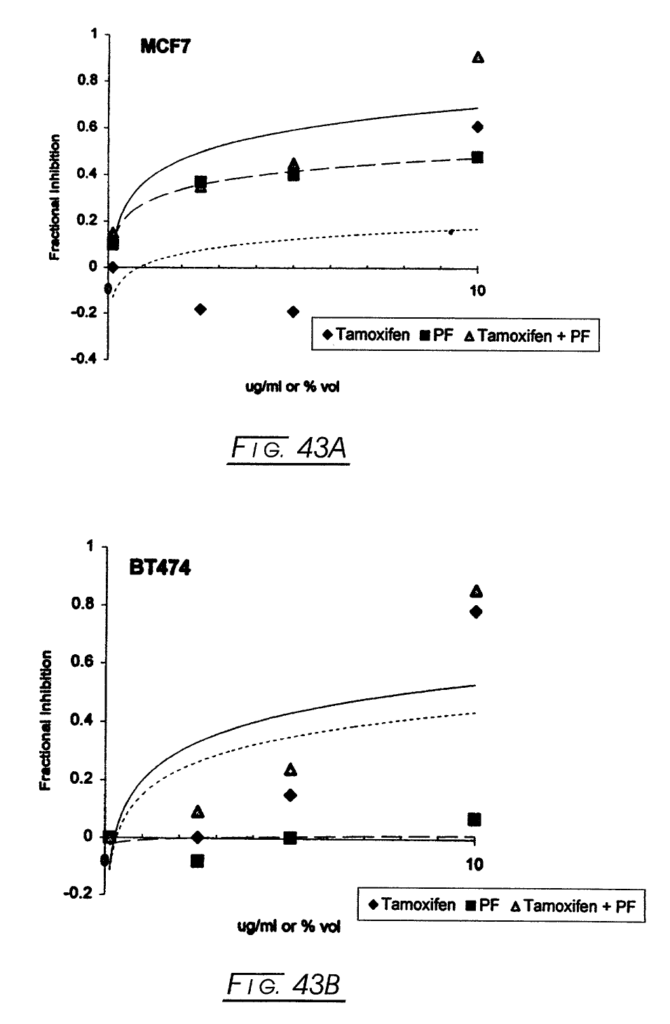

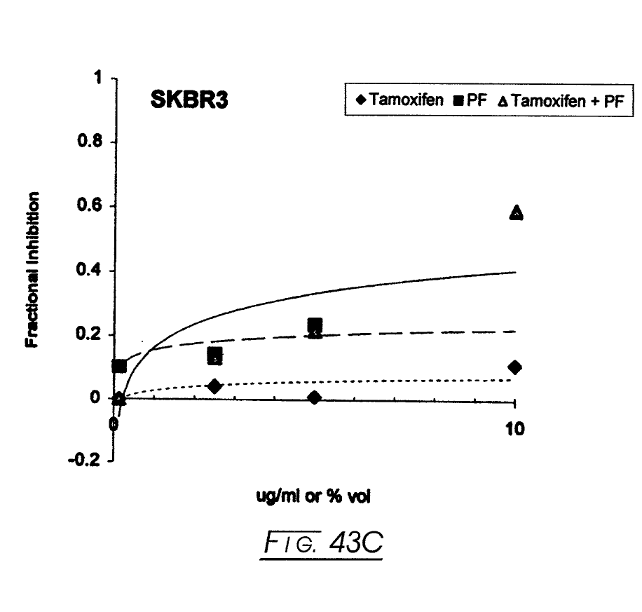

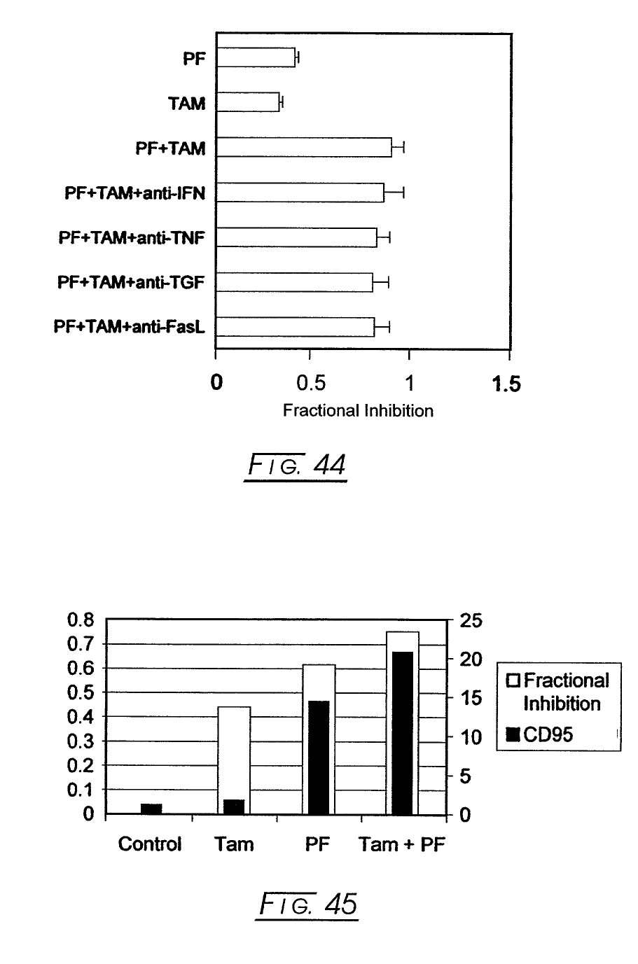

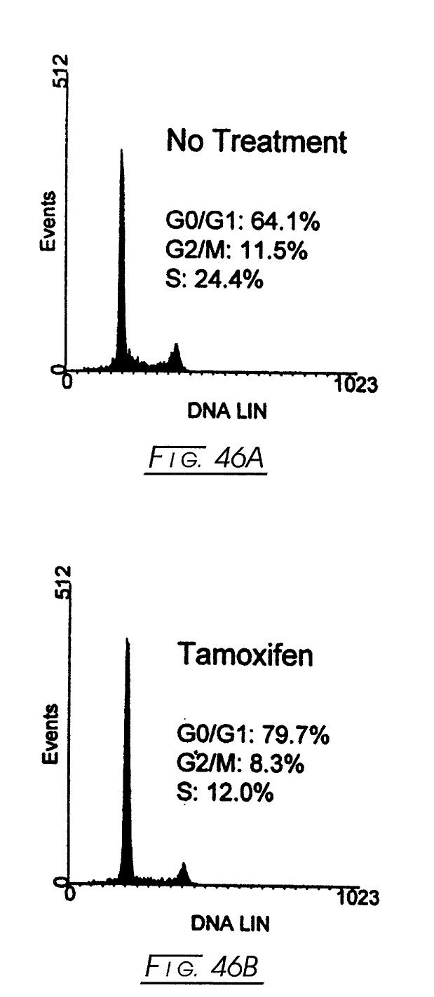

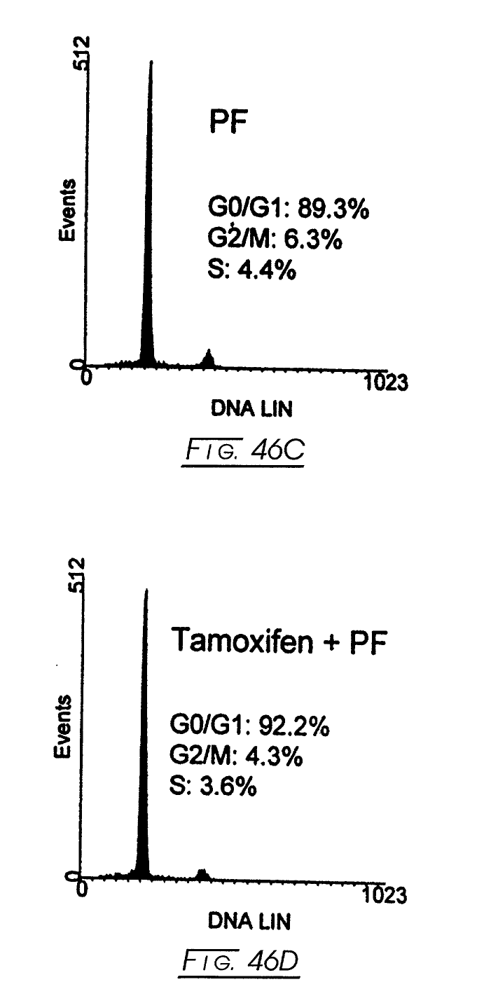

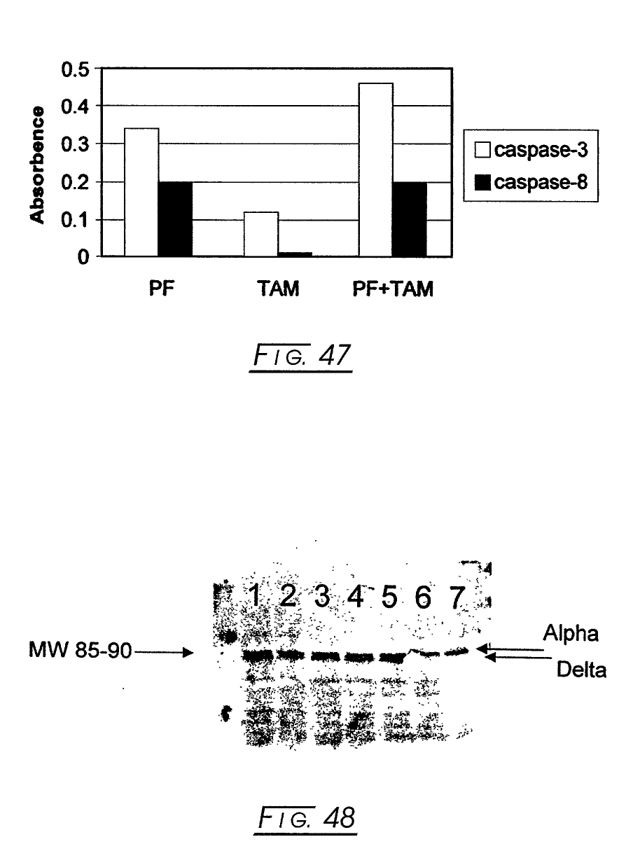

The combined effects of irinotecan and topotecan with the stimulated TRL supernatants were evaluated using median effect analysis. Synergistic interactions, with a Cl considerably less than 1.0 were observed in p53-wild-type LS513 and in p53 mutated SW480 colorectal cell lines. This data is presented below in Tables 17 (SW480 cells) and 18 (LS513 cells), and in FIGS. 25-28, respectively, for topotecan; and Tables 19 (SW480 cells) and 20 (LS513 cells), and in FIGS. 29-32, respectively, for irinotecan.

TABLE 17
SW480 Cells

| Cl Simulations | | | | | Mutually Non-Exclusive Cl Simulations | | | |
|---|---|---|---|---|---|---|---|---|
| | | | 5FU | SUP | | | 5FU | SUP |
| Fa | Cl | Est. s.d. | (μg/ml) | (%) | Fa | Cl | (μg/ml) | (%) |
| 0.05 | 0.834 | 3.377 | 0.00278 | 0.06953 | 0.05 | 0.844 | 0.00278 | 0.06953 |
| 0.1 | 0.693 | 1.9122 | 0.00655 | 0.16375 | 0.1 | 0.705 | 0.00655 | 0.16375 |
| 0.15 | 0.62 | 1.3609 | 0.01113 | 0.27829 | 0.15 | 0.634 | 0.01113 | 0.27829 |
| 0.2 | .0573 | 1.0671 | 0.01659 | 0.41487 | 0.2 | 0.588 | 0.01659 | .041487 |
| 0.25 | 0.538 | 0.883 | 0.02308 | 0.57695 | 0.25 | 0.554 | 0.02308 | .057695 |
| 0.3 | 0.51 | 0.7662 | 0.03078 | 0.76959 | 0.3 | 0.528 | 0.03078 | 0.76959 |
| 0.35 | 0.488 | 0.6848 | 0.03999 | 0.99976 | 0.35 | 0.507 | 0.03999 | 0.99976 |
| 0.4 | 0.469 | 0.6301 | 0.05108 | 1.27711 | 0.4 | 0.489 | 0.05108 | 1.27711 |
| 0.45 | 0.452 | 0.5953 | 0.0646 | 1.61505 | 0.45 | 0.474 | 0.0646 | 1.61505 |
| 0.5 | 0.438 | 0.5768 | 0.08131 | 2.03279 | 0.5 | 0.461 | 0.08131 | 2.03279 |
| 0.55 | 0.426 | 0.5722 | 0.10234 | 2.55857 | 0.55 | 0.45 | 0.10234 | 2.55857 |
| 0.6 | 0.415 | 0.5806 | 0.12942 | 3.23561 | 0.6 | 0.44 | 0.12942 | 3.23561 |
| 0.65 | 0.405 | 0.6024 | 0.16533 | 4.1332 | 0.65 | 0.433 | 0.16533 | 4.1332 |
| 0.7 | 0.398 | 0.6398 | 0.21478 | 5.36941 | 0.7 | 0.427 | 0.21478 | 5.36941 |
| 0.75 | 0.393 | 0.6975 | 0.28649 | 7.16219 | 0.75 | 0.424 | 0.28649 | 7.16219 |
| 0.8 | 0.391 | 0.7868 | 0.39841 | 9.96027 | 0.8 | 0.425 | 0.39841 | 9.96027 |
| 0.85 | 0.395 | 0.9338 | 0.59393 | 14.84836 | 0.85 | 0.432 | 0.59393 | 14.84836 |
| 0.9 | 0.412 | 1.2166 | 1.00939 | 25.2348 | 0.9 | 0.455 | 1.00939 | 25.2348 |
| 0.95 | 0.476 | 2.0117 | 2.37721 | 59.43021 | 0.95 | 0.529 | 2.37721 | 59.43021 |
| 0.97 | 0.556 | 3.0145 | 4.37279 | 109.3198 | 0.97 | 0.617 | 4.37279 | 109.3198 |

TABLE 18
LS513 Cells

| Cl Simulations | | | | | Mutually Non-Exclusive Cl Simulations | | | |
|---|---|---|---|---|---|---|---|---|
| | | | Topo | SUP | | | Too | SUP |
| Fa | Cl | Est. s.d. | (μg/ml) | (%) | Fa | Cl | (μg/ml) | (%) |
| 0.05 | 1.019 | 4.1385 | 0.00156 | 0.03901 | 0.05 | 1.161 | 0.00156 | 0.03901 |
| 0.1 | 0.731 | 1.8612 | 0.00394 | 0.09847 | 0.1 | 0.806 | 0.00394 | 0.09847 |
| 0.15 | 0.595 | 1.1593 | 0.00699 | 0.17469 | 0.15 | 0.646 | 0.00699 | 0.17469 |
| 0.2 | 0.51 | 0.8252 | 0.01076 | 0.26898 | 0.2 | 0.547 | 0.01076 | 0.26898 |
| 0.25 | 0.449 | 0.6346 | 0.01537 | 0.38419 | 0.25 | 0.478 | 0.01537 | 0.38419 |
| 0.3 | 0.401 | 0.5156 | 0.02098 | 0.52456 | 0.3 | 0.425 | 0.02098 | 0.52456 |
| 0.35 | 0.362 | 0.34383 | 0.02784 | 0.69604 | 0.35 | 0.382 | 0.02784 | 0.69604 |
| 0.4 | 0.33 | 0.3879 | 0.03628 | 0.90694 | 0.4 | 0.346 | 0.03628 | 0.92694 |
| 0.45 | 0.301 | 0.3559 | 0.04676 | 1.16894 | 0.45 | 0.315 | 0.04676 | 1.16894 |
| 0.5 | 0.275 | 0.3373 | 0.05996 | 1.49896 | 0.5 | 0.287 | 0.05996 | 1.49896 |
| 0.55 | 0.252 | 0.3287 | 0.07689 | 1.92215 | 0.55 | 0.262 | 0.07689 | 1.92215 |
| 0.6 | 0.23 | 0.3278 | 0.0991 | 2.47743 | 0.6 | 0.238 | 0.0991 | 2.47743 |
| 0.65 | 0.209 | 0.3332 | 0.12912 | 3.22807 | 0.65 | 0.216 | 0.12912 | 3.22807 |
| 0.7 | 0.189 | 0.3442 | 0.17133 | 4.28337 | 0.7 | 0.195 | 0.17133 | 4.28337 |
| 0.75 | 0.169 | 0.3611 | 0.23394 | 5.84841 | 0.75 | 0.174 | 0.23394 | 5.84841 |
| 0.8 | 0.149 | 0.3851 | 0.33413 | 8.35337 | 0.8 | 0.153 | 0.33413 | 8.35337 |
| 0.85 | 0.128 | 0.4198 | 0.51448 | 12.86211 | 0.85 | 0.13 | 0.51448 | 12.86211 |
| 0.9 | 0.104 | 0.475 | 0.91274 | 22.81843 | 0.9 | 0.106 | 0.91274 | 22.81843 |
| 0.95 | 0.075 | 0.5882 | 2.30398 | 57.5996 | 0.95 | 0.076 | 2.30398 | 57.5996 |
| 0.97 | 0.059 | 0.6902 | 4.45253 | 111.3132 | 0.97 | 0.06 | 4.45253 | 111.3132 |

TABLE 19
SW480 Cells

| Cl Simulations | | | | | Mutually Non-Exclusive Cl Simulations | | | |
|---|---|---|---|---|---|---|---|---|
| | | Est. | CPT | SUP | | | CPT | SUP |
| Fa | Cl | s.d. | (μg/ml) | (%) | Fa | Cl | (μg/ml) | (%) |
| 0.05 | 8.705 | 6.6855 | 0.00389 | 0.97128 | 0.05 | 12.215 | 0.389 | 0.97128 |
| 0.1 | 2.708 | 1.2787 | 0.07867 | 1.96684 | 0.1 | 3.519 | 0.07867 | 1.96684 |
| 0.15 | 1.389 | 0.5741 | 0.12177 | 3.04429 | 0.15 | 1.717 | 0.12177 | 3.04429 |
| 0.2 | 0.879 | 0.3794 | 0.16919 | 4.22984 | 0.2 | 1.045 | 0.16919 | 4.22984 |
| 0.25 | 0.626 | 0.2932 | 0.222 | 5.55009 | 0.25 | 0.72 | 0.222 | 5.55009 |
| 0.3 | 0.479 | 0.2434 | 0.28146 | 7.03658 | 0.3 | 0.537 | 0.28146 | 7.03658 |
| 0.35 | 0.386 | 0.2109 | 0.34916 | 8.72907 | 0.35 | 0.423 | 0.34916 | 8.72907 |
| 0.4 | 0.323 | 0.1885 | 0.42718 | 10.67955 | 0.4 | 0.347 | 0.42718 | 10.67955 |
| 0.45 | 0.277 | 0.1726 | 0.51832 | 12.95797 | 0.45 | 0.294 | 0.51832 | 12.95797 |
| 0.5 | 0.243 | 0.1611 | 0.62646 | 15.66139 | 0.5 | 0.254 | 0.621646 | 15.66139 |
| 0.55 | 0.216 | 0.1527 | 0.75715 | 18.92882 | 0.55 | 0.223 | 0.75715 | 18.92882 |
| 0.6 | 0.194 | 0.1464 | 0.91869 | 22.96718 | 0.6 | 0.198 | 0.91869 | 22.96718 |
| 0.65 | 0.175 | 0.1416 | 1.12396 | 28.0991 | 0.65 | 0.178 | 1.12696 | 28.0991 |
| 0.7 | 0.158 | 0.1378 | 1.39431 | 34.85774 | 0.7 | 0.161 | 1.39431 | 34.85774 |
| 0.75 | 0.144 | 0.1347 | 1.76775 | 44.19375 | 0.75 | 0.145 | 1.76775 | 44.19375 |
| 0.8 | 0.129 | 0.1318 | 2.31951 | 57.98779 | 0.8 | 0.13 | 2.31951 | 57.98779 |
| 0.85 | 0.115 | 0.1287 | 3.2228 | 80.5701 | 0.85 | 0.116 | 3.2228 | 80.5701 |
| 0.9 | 0.1 | 0.1249 | 4.98829 | 124.7072 | 0.9 | 0.1 | 4.98829 | 124.7072 |
| 0.95 | 0.008 | 0.1185 | 1.010129 | 252.5324 | 0.95 | 0.08 | 1.010129 | 252.5324 |
| 0.97 | 0.068 | 0.1137 | 16.68805 | 417 | 0.97 | 0.068 | 16.68805 | 417 |

TABLE 20
LS513 Cells

| Cl Simulations | | | | | Mutually Non-Exclusive Cl Simulations | | | |
|---|---|---|---|---|---|---|---|---|
| | | | 5FU | SUP | | | 5FU | SUP |
| Fa | Cl | Est. s.d. | (μg/ml) | (%) | Fa | Cl | (μg/ml) | (%) |
| 0.05 | 2.081 | 7.27 | 0.0319 | 0.07967 | 0.05 | 2.675 | 0.0319 | 0.07967 |
| 0.1 | 1.369 | 2.8428 | 0.00738 | 0.18448 | 0.1 | 1.34 | 0.00738 | 0.18448 |
| 0.15 | 1.057 | 1.6292 | 0.01241 | 0.31025 | 0.15 | 1.217 | 0.01241 | 0.31025 |
| 0.2 | 0.869 | 1.0976 | 0.01835 | 0.45887 | 0.2 | 0.979 | 0.01835 | 0.45887 |
| 0.25 | 0.74 | 0.8154 | 0.02536 | 0.634 | 0.25 | 0.821 | 0.02536 | 0.634 |
| 0.3 | 0.643 | 0.651 | 0.03364 | 0.84088 | 0.3 | 0.704 | 0.03364 | 0.84088 |
| 0.35 | 0.566 | 0.5512 | 0.04347 | 1.08675 | 0.35 | 0.614 | 0.04347 | 1.08675 |
| 0.4 | 0.502 | 0.4904 | 0.5526 | 1.38152 | 0.4 | 0.54 | 0.5526 | 1.38152 |
| 0.45 | 0.448 | 0.4541 | 0.06956 | 1.73901 | 0.45 | 0.478 | 0.06956 | 1.73901 |
| 0.5 | 0.4 | 0.4338 | 0.08716 | 2.17888 | 0.5 | 0.425 | 0.08716 | 2.17888 |
| 0.55 | 0.358 | 0.4243 | 0.1092 | 2.73002 | 0.55 | 0.278 | 0.1092 | 2.73002 |
| 0.6 | 0.319 | 0.4221 | 0.13746 | 3.43645 | 0.6 | 0.335 | 0.13746 | 3.43645 |
| 0.65 | 0.283 | 0.4254 | 0.17474 | 4.36856 | 0.65 | 0.296 | 0.17474 | 4.36856 |
| 0.7 | 0.249 | 0.4332 | 0.22584 | 5.6459 | 0.7 | 0.259 | 0.22584 | 5.6459 |
| 0.75 | 0.217 | 0.4453 | 0.29953 | 7.48825 | 0.75 | 0.224 | 0.29953 | 7.48825 |
| 0.8 | 0.184 | 0.4624 | 0.41384 | 10.34606 | 0.8 | 0.19 | 0.41384 | 10.34606 |
| 0.85 | 0.152 | 0.4868 | 0.61209 | 15.30228 | 0.85 | 0.156 | 0.61209 | 15.30228 |
| 0.9 | 0.117 | 0.5249 | 1.0294 | 25.73512 | 0.9 | 0.12 | 1.0294 | 25.73512 |
| 0.95 | 0.077 | 0.5999 | 2.38365 | 59.59125 | 0.95 | 0.078 | 2.38365 | 59.59125 |
| 0.97 | 0.057 | 0.6643 | 4.33215 | 108 | 0.97 | 0.058 | 4.33215 | 108 |

For irinotecan, synergism was primarily observed at high levels of antiproliferative effects. Synergism for topotecan was observed across all levels. The enhanced cytotoxicity was associated with an increase in the expression of FasR (see FIG. 24). The combination of TRL soluble products and drug led to an increase in the activity of caspase-3. Caspase-8 was induced with the TRL supernatant, but did not increase with the combination of TRL supernatant and topoisomerase-I drug (see FIG. 24).

The role of TNF-α and IFN-γ, cytokines previously reported to modulate camptothecin activity, and soluble FasL in the effects observed was examined in LS513 cells using blocking antibody. The results are displayed in Table 21 and FIG. 33.

TABLE 21

| | |
|---|---|
| Blocking Antibody | Fractional Inhibition |

| | | |
|---|---|---|
| Sup + TPT + anti-IFN | 0.81 | 0.08 |
| Sup + TPT + anti-TNF | 0.785 | 0.07 |
| Sup + TPT + anti-FasL | 0.69 | 0.03 |
| Sup + TPT | 0.84 | 0.02 |
| TPT | 0.51 | 0.015 |
| Sup | 0.2 | 0.015 |

The addition of anti-FasL antibody partially abrogated the enhanced antiproliferative effects observed with the supernatant-topotecan combination. Anti-TNF-α and anti-IFN-γ blocking antibody had no effect. The ability of the supernatants to enhance topotecan chemosensitivity persists for at least 72 hours after exposure (FIG. 34) and is reduced somewhat by 96 hours.

Effect of Factor on Topoisomerase I-DNA Complexes

LS513 cells were suspended in media or media plus 25% purified factor (or no additions) for 1, 12, 24, and 48 hours, with or without 50 μM irinotecan (CPT). Three different concentrations of DNA (recovered from the DNA peak fractions of each CsCI gradient) were spotted onto the membrane.

Untreated cells (four different time points) and 25% media negative controls had very low, ie., basal, levels of topoisomerase I complexes, as expected. A positive control (camptothecin or CPT) clearly showed trapping of the covalent complexes at all times of exposure. Mixing CPT with media only gave topoisomerase I signals that were identical to that seen with CPT alone; however, addition of the purified factor at 25% yielded a reproducible increase in topoisomerase I/DNA complexes at 24 and 48 hours. The blot was placed on a phosphorimager and signal strengths from the 2 μg slots were quantified. Based upon comparison to a known standard of purified topoisomerase I on the same blot, the signals are expressed as ng or topoisomerase I per ml of DNA=no additions (i.e., untreated). The data are quantified in Table 22 and FIG. 35.

TABLE 22

| Time (hrs) | NA | Media | Factor | CPT11 | CPT11 + Media | CPT11 + Factor |
|---|---|---|---|---|---|---|
| 1 | 3.43 | 3.36 | 3.93 | 3.93 | 4.17 | 3.22 |
| 12 | 2.98 | 3.29 | 3.35 | 4.83 | 5.03 | 4.64 |
| 14 | 3.55 | 3.23 | 3.41 | 4.32 | 5.2 | 7.43 |
| 48 | 3.72 | 3.55 | 3.39 | 5.37 | 4.71 | 5.97 |

Since the assay is monospecific for topoisomerase I, these results clearly demonstrate that the factor enhances DNA damage driven by topoisomerase I. The factor enhances the formation of topoisomerase I-DNA complexes.

### Example 4

### Interface of Cancer And HIV-1

Experimental

The purpose of this experiment was to demonstrate that Factor C that inhibits the replication of HIV-1 in vitro are present in the supernatants of lymph node lymphocytes (LNL) expanded from HIV+ donors. To that end, PB CD4+ T lymphocytes were isolated from a normal volunteer using negative selection (Human T Cell CD4 Subset Column Kit, R&D Systems, Inc., Minneapolis, Minn.). These purified CD4+ cells were activated with 10 ng of OKT3/ml and grown in RPMI-1640 medium supplemented with 10% fetal bovine serum and 100 IU of IL-2/ml. Cells were maintained between 0.5 and 2×10⁶/ml by addition of fresh complete medium approximately once per week. CD4+ T lymphocytes (5×10⁵/ml/well) were added to a 24-well plate containing either 20% or 805 of supernatant from LNL cultures expanded form HIV+ donors using 5, 20, or 100 ng of OKT3/ml. Control wells were established containing 20% or 80% fresh medium or supernatant from an LNL culture expanded from an HIV-colorectal cancer patient activated with 100 ng of OKT3/ml. All wells, except no virus control wells, were infected with HIV+ culture supernatant known to contain sufficient HIV to infect lymphocyte cultures at proportions used.

Supernatants were collected from the 24-well plate at twice weekly intervals. After each collection, the wells were re-fed with the same proportions of supernatants from the same original LNL cultures as before. Day 4 and day 12 supernatants from the 24-well plate were analyzed by quantitative ELISA for HIV-1 p24 antigen (Coulter, Hiyalea, Fla.), as were the supernatants from the original LNL cultures. The data were collected and analyzed by subtracting the p24 present in the 20% or 80% of the original HIV+ LNL culture supernatants from the p24 detected in the 24-well plate wells—this is the amount of p24 produced. The p24 produced in the control wells with fresh medium alone was compared to the p24 produced in wells with 20% or 80% supernatant from the original HIV+ LNL cultures or from the control cancer patient LNL culture.

Results

All wells infected with HIV were highly positive for p24 antigen at both day 4 and day 12. Wells not infected with virus had no p24 antigen. Supernatants from LNL cultures from HIV+ donors inhibited the replication of HIV in the normal CD4+ lymphocytes infected with HIV in vitro as set forth in Table 23 below and in FIGS. 36-39.

TABLE 23
PERCENT HIV REPRESSION (p24)*

| | Day 4 | | Day 12 | |
|---|---|---|---|---|
| | 20% | 80% | 20% | |
| | LNL Sup | LNL Sup | LNL Sup | 80% LNL Sup |
| HIV+ Patient 1 | −6 | 22 | 16 | 56 |
| (5 ng/ml OKT3) | | | | |
| HIV+ Patient 1 | −56 | 22 | 17 | 55 |
| (20 ng/ml OKT3) | | | | |
| HIV+ Patient 1 | −53 | 44 | 27 | 78 |
| (100 ng/ml OKT30 | | | | |
| HIV+ Patient 2 | −13 | 9 | 16 | 29 |
| (5 ng/ml OKT3) | | | | |
| HIV+ Patient 2 | 0 | 21 | 91 | 90 |
| (100 ng/ml OKT3) | | | | |
| Cancer Patient | −13 | −5 | 4 | 92 |
| (100 ng/ml OKT30 | | | | |
| No LNL Sup | 0 | 0 | 0 | 0 |
| (medium control) | | | | |
| No Virus Control | 99 | 101 | 99 | 100 |

*Sup is supernatant

No inhibition of HIV replication was seen at day 4 using 20% LNL supernatants form HIV+ donors or the cancer patient, as seen in FIG. 36. HIV replication, as seen on day 4, was inhibited by the presence of 80% supernatants form HIV+ LNL cultures, but not by the cancer patient LNL supernatant, as seen in FIG. 37. By day 12, the presence of 20% HIV+ LNL culture supernatants, but not the cancer patient LNL supernatant, inhibited HIV replication, as seen in FIG. 38. However, HIV replication by day 12 was inhibited by the presence of 80% supernatants from both HIV+ LNL cultures and cancer patient LNL cultures, as seen in FIG. 39. Furthermore, wherever HIV repression was detected, it was always greater when using the supernatants from LNL activated with the highest amount (100 ng/ml) of OKT3.

The obvious unexpected result reported above is that the cancer patient supernatant inhibited HIV replication. Based on the disclosure herein, however, it is apparent that Factor C of the present invention has a wide activity range.

### Example 5

Herpes Simplex Virus (Strain KOS) and Coxsackie Virus B3

Viruses

The ampoule containing Herpes Simples Virus (HSV), strain KOS, was thawed and its contents diluted 1:100,000 in DMEM and 0.5 ml was added to confluent monolayer of VERO cells (African Green Monkey Kidney). HSV was adsorbed over 30 minutes at 37° C. with rocking. The cells then were incubated in 25 ml of Dulbecco's Modified Minimal Essential Medium (with Earles Salts), with 2% heat-inactivated fetal bovine serum (FBS), Na Pyruvate, and supplemented with 100 IU penicillin and 50 μg/ml streptomycin (Maintenance Medium or MM) at 37° C. and 5% CO₂ for 3 days. Cells were frozen and thawed 1× and debris clarified by 200× g centrifugation for 10 minutes. Supernatants were placed in ampoules in 1.0 ml aliquots, labeled “HSV POOL1, date” and “HSV POO1, date”, and stored at −80° C. until used.

The ampoule containing Coxsackie virus B3 (ATCC VBR-30, strain Nancy) was thawed rapidly at 37° C., diluted in 1:100 in DMEM, and 0.1 ml added to a T75 flask containing LL-C-Mk2 cells. The virus was adsorbed for 30 minutes with rocking at 37° C., and 20 ml of DMEM+2% FBS was added. CPE was noted in 48 hours and the virus was harvested and placed in ampoules the next day, stored at '80° C. in 1.0 ml aliquots.

Virus Plague Titration

Coxsackie virus was titrated in LLC-mk-2 cells. HSV was titrated in VERO MONKEY kidney cells. All cells were grown in 6 well Co-Star plates. At confluency, the medium was aspirated and infected with virus. Stock virus was diluted 10 fold in cold DMEM from for 9 days.

Four wells per dilution were infected with 0.1 ml diluted virus and adsorbed for 30 minutes at 37° C. at the end of the incubation period, the wells were overlaid with an equal mixture of 0.3% methylcellulose and DMEM+FBS. For the Coxsackie virus, plates were incubated 48 hours, then 2 ml of an 0.3% Neutral Red solution was added to each well. For HSV, the plates were incubated 72 hours and 2.0 ml of an 0.3% Neutral Red solution was added to each well. 24 hours later, all media was aspirated and plaques enumerated in a darkened room over a white light source. The plaques ranged in size from approximately 1 mm to 4-5 mm in size, depending upon the virus used. Titers are expressed in Plaque-Forming Units (PFU) per 0.1 ml.

Experimental Plaque Reduction Assays

In order to determine if a fractionated supernatant from a colorectal cancer patient could inhibit viruses other than HIV as reported in Example 4, 0.1 ml of the >50,000 and <50,000 dalton fractions of activated-expanded supernatant from an HIV-colorectal cancer patient, and the whole activated-expanded supernatant itself were added to confluent monolayers in the Co-Star plates (n+12) after the growth medium was aspirated. Growth medium served as the control supernatant. The plates were incubated, with periodic rocking, at 37° C. in 5% CO₂ atmosphere for 30 minutes. At the end of the incubation period, the supernatants were aspirated and the cells infected with 40-80 calculated plaque forming units (PFU)/0.1 ml. The virus was adsorbed for 30 minutes at 37° C. in a 5% CO₂ atmosphere. With periodic rocking to assure even distribution of the virus. At the end of the incubation period, each well was overlaid with equal volumes of methylcellulose and DMEM medium supplemented with FBS, and incubated 2 days for the Cosackie virus and 3 days for HSV, at which time 2.0 ml of Neutral Red was added to each well. Cultures were incubated for 24 hours more, the medium aspirated, and the plaques enumerated over a light box in a darkened room. Plaque reduction was determined according to the following formula:

$$\frac{[ Control - Experimental ]}{Experimental} \times 100 = Percent Reduction$$

Results

The following results recorded for HSV are set forth in Table 24.

TABLE 24

| Material | No. of Points | S.D. | Mean | t-tail |
|---|---|---|---|---|
| >50 k fraction | 6 | 9.7039 | 42.167 | 0.0022 |
| <50 k fraction | 6 | 11.0045 | 111.500 | 0.4849 |
| Whole sup. | 6 | 9.2664 | 23.666 | 0.0022 |
| Medium control | 6 | 18.1291 | 120.333 | N/A |

Compared to the medium control, the percent reduction in PFU is as follows:

| | |
|---|---|
| >50 k fraction | 65% |
| <50 k fraction | 7% |
| Whole supernatant | 80% |

These results demonstrate that the >50 k fraction displayed significant anti-viral activity against HSV.

The following results recorded for Coxsackie Virus are set forth in Table 25.

TABLE 25

| Material | No. of Points | S.D. | Mean | t-tail |
|---|---|---|---|---|
| >50 k fraction - 1 | 9 | 8.14 | 18.44 | 0.0004 |
| >50 k fraction - 2 | 6 | 9.28 | 46.83 | 0.0022 |
| <50 k fraction - 1 | 9 | 11.3 | 32.11 | 0.0048 |
| <50 k fraction - 2 | 6 | 7.73 | 77.66 | 1.0628 |
| Whole sup. - Whole | 9 | 4.21 | 22.67 | 0.0004 |
| Whole sup. - 1 | 6 | 8.9 | 49.17 | 0.0022 |
| Medium control | 9 | 5.15 | 48.56 | N/A |

Compared to the medium control, the percent reduction in PFU is as follows:

| | Run 1 | Run 2 |
|---|---|---|
| >50 k fraction | 62% | 41% |
| <50 k fraction | 33.9% | 1.5% |
| Whole supernatant | 53.3% | 37.6%% |

These results demonstrate that the >50 k fraction displayed significant anti-viral activity against Coxsackie virus.

### Example 6

### Oncology Data with Tamoxifen

Cell Lines and Reagents

Human breast carcinoma cells lines, MCF7, SKBR3, and BT474, were obtained from the American Type Culture Collection (ATCC, Rockville, Md.). Cells were cultured at 37° C. in 5% CO₂ in their maintenance media, which consisted of RPMI-1640 with 2 mM glutamine and 10% fetal bovine serum (FBS; Gibco BRL, Grand Island, N.Y.). Anti-TNF, anti-lFN-γ, anti-RGF-β (R&D Systems, Minneapolis, Minn.), anti-Fas ligand (FasL; NOK1, PharMinegen), anti-Fas (CD95) (Coulter Corporation, Miami, Fla.), anti-protein kinase C alpha and delta antibodies (Santa Cruz Biotechnologies, Santa Cruz, Calif.), were obtained from commercial sources.

Cell Culture and Factor Production

Peripheral blood lymphocytes were activated and expanded with anti-CD3 mAb and IL-2 in serum-free medium in 5% CO₂ in humidified air at 37° C. as described by Triozzi, et al., “Adoptive immunotherapy using lymph node lymphocytes localized in vivo with radiolabeled monoclonal antibody”, J Natl Cancer Inst, 87:1180-1181 (1995). Day 10 cells were harvested by centrifugation (25033 g, room temperature, 6 min) in 50-ml tubes. The pelleted cells then were resuspended at 1.5×10⁶/ ml. Harvested cells were separated into purified CD8+ and CD4+ cells using anti-CD8 coated plastic flasks (Cellector-8, AIS, Santa Clara, Calif.) according to the recommendation of the manufacturer. CD4+ cell contamination of the Cd8+ cells was less than 2.0%; CD8+ cell contamination of the CD4+ cells was less than 2.0% (as determined by flow cytometry). The cells then were put into T-175 flasks pre-coated with OKT3 at a final volume of 200 ml per flask with 100 ng/ml of each antibody. Cells were cultured for 24 hours at 37° C. in 5% CO₂, and supernatants were collected by centrifugation at 400× g for 10 minutes.

Supernatant Fractionation

Supernatants were separated into fractions greater and less than 50 kDa by centrifugation at 10033 g for 30 minutes in Millipore Ultrafree Biomax (Bedford, Mass.) filter devices with nominal 50 kDa limits. Supernatants then were collected and diluted in expansion media. Supernatants also were subjected to sequential Superose 12 sizing and DEAE anion exchange chromatography. Two liters of supernatants were prepared for column chromatography by adding phenylmethyl sulfonyl fluoride and glycerol to 0.1% weight/volume. Supernatant was re-centrifuged for 30 minutes at 1000 g to remove remaining particulates. Supernatant then was loaded onto a 120 ml bed volume of Superose 12 column (Pharmacia) at 10 ml/minute. Unbound protein was rinsed off with 2 bed volumes of 8% % α-D-mannopyranoside in phosphate buffered saline. Peak fractions are pooled and dialyzed against 10 volumes of 20 mM Hepes buffer 0.1 % glycerol, pH 8.2, overnight at 4° C., using SpectrumPor CE Membrane with a 50,000 molecular weight cut-off. This is applied to DEAE Sepharose equilibrated with 20 mM Hepes, pH 8.2. Bound protein is eluted with a step gradient of 200 and 500 nM NaCl in Hepes buffer. Protein is concentrated using Millipore Ultrafree centrifugal filter devices, 50,000 molecular weight cut-off, and re-suspended in media for bioassay.

Tumor Proliferation Assay

Supernatants and their fractions were added at a range of volumes to the tumor cell lines or cultured in their maintenance media in 24-well plates for 96 hours. MTS was added for the final 5 hours of culture as recommended by the manufacturer (CeiTiter 96 AQᵤₑₒᵤₛ, Non-Radioactive Cell Proliferation Assay; Promega, Madison, Wis.), and the absorbence was read in a DigiScan reader (ASYS Hitech, Austria) at A₄₉₂ nm. Samples were evaluated in triplicate. Tumor cells cultured in maintenance media with no addition and the maintenance media alone were used as controls. Fractional inhibition was determined by the formula set forth above.

Flow Cytometry

Flow cytometry was used to assess Fas expression and cell cycle. Cells were reacted sequentially with mAb to Fas (CD95) and then with a fluorescenated goat anti-mouse antibody, according to the recommendations of the manufacturer. Percent fluorescent cells and fluorescence intensity was determined using an Epics Elite cytofluorograph (Coulter Corp.). All samples were compared to their isotype-matched controls. Analysis of cell cycle was performed using propidium iodide on a Coulter EPICS Elite flow cytometer equipped with a 488 nm, 15 mW, air-cooled Argon laser. (see Darzynkiewicz, et al., Methods in Cell Biology: Flow Cytometry, 2^{nd} Edition, Part A, pp 32-36, 1995). Optical laser alignment calibration of the flow cytometer was performed using Coulter's DNA-Check EPICS alignment fluorosphere beads with coefficient of variations routinely less than 2%. PI fluorescent light emission was collected through a 610 nm, long-pass transmission filter. PI signal was measured in linear mode and extended analysis of DNA content was performed using ModFit LT program, as described above. Data are presented as the percentage of cells in G1-G0, S, and G2-M.

Caspase Activity

Caspase-3 and caspase-8 activities were determined using a calorimetric assay kit (R&D Systems, Inc., Minneapolis, Minn.). Assays were conducted according to the instructions of the manufacturer. Protein kinase C, Prote3in kinase c alpha and delta levels were determined by immunoprecipitation and Western blotting, as previously described. Hofmeister, et al., “Clustered CD20 induced apoptosis: src-family kinase, the proximal regulator of tyrosine phosphorylation, calcium influx, and caspase 3-dependent apoptosis”, Blood Cells Mol Dis, 26:133-43, 2000.

Statistical Analysis

The combined effects of the drugs and supernatants were analyzed by the median effect method of Chou and Talalay using CalcuSyn software (see above). In brief, when two agents are administered at a fixed ratio, a combination index (Cl) is calculated depending on whether the drugs are assumed to be mutually non-exclusive or mutually exclusive in their action (see Chou, et al., “Quantitative analysis of dose-effect relationships: the combined effects of multiple drugs or enzyme inhibitors”, Adv Enzyme Regul, 22:27-55, 1984). According to this method, “synergy” is indicated by a Cl of less than 1, “addition” by a Cl equal to 1, and “antagonism” by a Cl greater than 1. A Cl of less than 0.3 is considered to represent “strong synergism”, and a Cl greater than 3.3 is considered to represent “strong antagonism”.

### Results

Soluble Product of Activated-expanded T-cells Have Antiproliferative Activity and Enhance the Antiproliferative Activity of Tamoxifen

T cells were activated and expanded with anti-CD3 mAb and IL-2 as described by Kim, et al., “Expansion of mucin-reactive lymph node lymphocyte subpopulations form patients with colorectal cancer”, Cancer Biother, 10:115-123, 1995). The cells that result from the culture regimen express mRNA for FasL and other members of the TNF family, including TNF-β and TRIAL, as well as IFN-γ, IL4, granulocyte-macrophage colony stimulating factor (GM-CSF), and transforming growth factor-β (TGF-β). They have been shown to secrete TNF-α, sFasL, IFN-γ, IL-4, GN-CSF, and TGF-β in response to tumor. The activated-expanded T cells generated do not produce significant IL-1α nor IFN-α. Berdel, et al., “Effects of hematopoietic growth factors on malignant nonhematopoietic cells”, Seminars in Oncology, 19 (Suppl 4):41-45, 1992); and Uhm, et al., “Modulation of transforming growth factor-b1 effects by cytokines”, Immunological Investigations, 22:375-388, 1993). Cytokines such as TFG-β, IL4, and IL-6 have been reported to inhibit breast cancer cell growth. Chen, et al., “Growth inhibition of human breast carcinoma and leukemia/lymphoma cell lines by recombinant inteferon-β₂”, Proc Natl Acad Sci USA, 85:8037-8041, 1988; Toi, et al., “Inhibition of colon and breast carcinoma cell growth by inteleukin-4”, Cancer Res, 52:275-279, 1992; and Artgea, et al., “Transforming growth factor β: potential autocrine growth inhibitor of estrogen receptor-negative human breast cancer cells”, Cancer Res, 48:3898-3904, 1988. Cytokines such as GM-CSF have been shown to stimulate growth of breast cancer cells. Dedhar, et al., “Human granulocyte-macrophage colony-stimulating factor is a growth factor active on a variety of cell types of nonhemopoietic origin,” Proc Natl Acad Sci USA, 85:9253-9257; Berdel, et al., “Various human hematopoietic growth factors (IL-3, GM-CSF, G-CSF) stimulate clonal growth of nonhematopoietic tumor cells,” Blood, 73:80,1989; Freiss, et al., “Control of breast cancer growth by steroids and growth factors: interactions and mechanisms”, Breast Cancer Res Treat, 27:57-68, 1993. Although both growth inhibitory and stimulatory cytokines are present, the net effect of the supernatants from the activated-expanded T cells is to inhibit growth.

The combined effects of the soluble products and tamoxifen were examined by culturing SKBR3 cells with a range of concentrations of the supernatants and tamoxifen at 10 μg/ml. Table 26 and FIG. 40 display the growth inhibitory effects of a range of concentrations of the supernatants of the activated-expanded T cells on SKRB3 breast cancer cells.

TABLE 26

| | | |
|---|---|---|
| Supernatant | | |
| Concentration | Supernatant | Supernatant + Tamoxifen |

| | | | | |
|---|---|---|---|---|
| 0 | 0 | 0 | 0.23 | 0.02 |
| 0.25 | 0.041 | 0.01 | 0.26 | 0.02 |
| 2.5 | 0.16 | 0.04 | 0.31 | 0.04 |
| 6.25 | 0.24 | 0.08 | 0.41 | 0.06 |
| 12.5 | 0.3 | 0.08 | 0.67 | 0.07 |
| 25 | 0.41 | 0.06 | 0.89 | 0.07 |

Enhanced antiproliferative activity was observed. The activated-expanded T cells were separated into CD4+ and CD8+ populations. Both populations produced the inhibitory soluble factors.

The Antiproliferative Activity and the Tamoxifen Enhancing Activity are Mediated by a Factor of Greater than 50 kDa

Most cytokines that have been shown to modulate breast cancer cell growth, such as IFNα (19 kDa), IFN-γ (8 kDa), and TGF-β (25 kDa), are less than 50 kDa. Members of the TNF family produced by activated T cells can exist as monomers in soluble form, including TNF-α (25 kDa) and FasL (27 kDa). Soluble members of the TNF family also can exist as trimers of greater than 50 kDa, including TNF-α(approximately 50 kDa) and FasL (70 to 80 kDa). Tanaka, et al., “Expression of the functional soluble form of human Fas ligand in activated lymphocytes”, EMBO J. 14:1129-1135,1995; and Yoshimura, et al., “Molecular weight of tumor necrosis factor determined by gel permeation chromatography alone or in combination with low-angle laser light scattering”, Biochemistry International, 17:1157-63, 1988.

Supernatants generated from activated-expanded CD4+ cells were separated into fractions of greater than 50 kDa and of less than 50 kDa. Most of the soluble FasL was in the >50 kDa fraction (500 pg/ml versus 110 pg/ml in the <50 kDa fraction). Most of the TNF-α was in the <50 kDa fraction (400 pg/ml versus 50 pg/ml in the >50 kDa fraction). All of the IFN-γ (420 pg/ml) was in the <50 kDa fraction. Tumor cells were cultured in unfractionated supernatant, a >50 kDa fraction, and a <50 kDa fraction. The unfractionated and the >50 kDa fraction demonstrated antiproliferative activity associated alone and in combination with tamoxifen. The <50 kDa fraction did not. This data is presented in Table 27 and FIG. 41.

TABLE 27

| | | |
|---|---|---|
| Culture | F1 | CD95 |

| | | | | |
|---|---|---|---|---|
| Control | 0.01 | 0.001 | 1 | 0.1 |
| Supernatant | 0.39 | 0.022 | 15 | 0.8 |
| Tamoxifen | 0.28 | 0.019 | 11 | 0.9 |
| Supernatant + Tamoxifen | 0.61 | 0.049 | 25 | 1 |
| <50 kDa Fraction | 0.014 | 0.016 | 0 | 0 |
| <50 kDa Fraction + Tamoxifen | 0.33 | 0.018 | 9 | 0.8 |
| >50 kDa Fraction | 0.42 | 0.15 | 14 | 1 |
| >50 kDa Fraction + Tamoxifen | 0.81 | 0.045 | 31 | 2 |

Supernatants derived from the >50 kDa fraction produced by activated-expanded CD4+ cells were subjected to Superose 12 sizing and then DEAE anion exchange. Fractions were screened for activity, which was isolated to a fraction containing a protein that on SDA-PAGE existed at approximately 70 kDa, as can be seen in FIG. 42. Two bands, seen at approximately 23 and 46 kDa on this gel, performed under reducing conditions. The combined effects of tamoxifen with purified Factor C were evaluated in ER-positive MCF-7 and BT474 cells and ER-negative SKBR3 cell lines. This data is displayed in FIG. 43. Median effect analysis was used to analyze the interactions. Synergistic interactions, with a Cl of considerably less than 1.0, were observed in all 3 cells lines.

The role of TNF-α, TGF-β, and IFN-γ cytokines, previously reported to modulate tamoxifen activity, and FasL in the effects observed, was examined in SKBR3 cells using blocking antibody. These results are reported in Table 28 and FIG. 44.

TABLE 28

| | |
|---|---|
| Culture | Fractional Inhibition |

| | | |
|---|---|---|
| Factor C | 0.4 | 0.015 |
| Tamoxifen | 0.32 | 0.015 |
| Factor C + Tamoxifen | 0.9 | 0.06 |
| Factor C + Tamoxifen + anti-FasL | 0.81 | 0.08 |
| Factor C + Tamoxifen + anti-TGF | 0.8 | 0.08 |
| Factor C + Tamoxifen + anti-TNF | 0.82 | 0.07 |
| Factor C + Tamoxifen + anti-IFN | 0.86 | 0.1 |

Blocking antibody to these cytokines had no effect on the interaction.

Factor C Combined with Tamoxifen Enhances Apoptosis, Increases Fas, Induces Cells into G0/G1. Increases Caspase 3 and 8. and Modulates Protein Kinase C Soluble products of immune cells and tamoxifen have been reported to induce Fas-mediated apoptosis. Morphologic changes typical of apoptosis, including membrane bleeding and chromatin condensation, are apparent as early as 24 hours after exposure to Factor C and tamoxifen. The enhanced antiproliferative effect is associated with an increase in the expression of Fas. This can been in Table 29 and FIG. 45.

TABLE 29

| Culture | Fractional Inhibition | CD 95 Cells |
|---|---|---|
| Control | 0 | 1.4 |
| Tamoxifen | 0.445 | 1.8 |
| Factor C | 0.619 | 14.5 |
| Factor C + Tamoxifen | 0.755 | 20.8 |

Tumor cells are induced into G0/G1 of the cell cycle, as can been from FIG. 46.

Apoptosis is mediated by proteases of the caspase family. The activity of capase-3, a “downstream” executioner caspase in the apoptotic pathway, and caspase-8, a more proximal caspase that is triggered by FasL and other members of the TNF family was evaluated. See Boldin, et al., “Involvement of MACH, a novel MORT1/FADD-interacting protease, in Fas/APO-1 and TNF receptor-induced cell death”, Cell, 85:803,1996. These data are presented in Table 30 and FIG. 47.

TABLE 30

| Culture | Caspase-3 | Caspase-8 |
|---|---|---|
| Tamoxifen | 0.12 | 0.01 |
| Factor C | 0.34 | 0.2 |
| Factor C + Tamoxifen | 0.46 | 0.2 |

Caspase-3 was induced with Factor C and increased with the combination of Factor C and tamoxifen. Caspase-8 was induced with Factor C, but did not increase with the Factor C-tamoxifen combination.

Protein kinase C has been implicated in the antiproliferative activity of tamoxifen in ER-negative cells. Growth inhibition of prostate cancer cells is not dependent upon estrogenic activity, but is associated with inhibition of protein kinase C and activation of the TGF-β signaling pathway, including induction of the cell cycle-inhibitory protein, p₂₁^{wafl/cip1}. Tohlff, et al., “Prostate cancer cell growth inhibition by tamoxifen is associated with inhibition of protein kinase C and induction of p21wafl/cp1”, Prostate, 37:51-59, 1998. Tamoxifen induces selective membrane association of protein kinase C epsilon in MCF-7 cells. Lavie, et al., “Tamoxifen induces selective membrane association of protein kinase C epsilon in MCF-7 human breast cancer cells”, Int J Cancer, 77:928-932, 1998. The inhibition of protein kinase C may be related to the cationic amphiphilic nature of tamoxifen. Friedman, “Tamoxifen and vanadate synergize in causing accumulation of polyphosphoinositide in Gh₄C₁ membranes”, J Pharmacol Exp Ther, 267:617-623, 1993. The combined effects of Factor C and tamoxifen on protein kinase C alpha and delta were evaluated. As can be seen in FIG. 48, inhibition of protein kinase C delta was seen with Factor C and with Factor C combined with tamoxifen.

1. A factor for treating patients afflicted with a disease that leads to an immunosuppressed state in the patient, which comprises:
≧50 kDa fraction of a supernatant derived from lymphocyte cells, which have been subjected to mitogenic stimulation in serum free medium.

2. The factor of claim 1, wherein said lymphocyte cells are lymph node lymphocyte cells.

3. The factor of claim 1, wherein said mitogenic stimulation includes the presence of lnterleukin-2 (IL-2) and anti-CD3 monoclonal antibody.

4. The factor of claim 3, wherein said mitogenic stimulation includes the presence of IL2 and soluble anti-CD3 monoclonal antibody followed by re-stimulation in the presence of insoluble anti-CD3 monoclonal antibody.

5. The factor of claim 1, wherein said lymphocyte cells are peripheral blood lymphocyte cells.

6. The factor of claim 1 wherein said factor comprises an approximately 70-80 kDa fraction of said 50 kDa fraction.

7. The factor of claim 4, which comprises an approximately 70-80 kDa fraction thereof.

8. The factor of claim 4, wherein said lymphocyte cells are derived from one or more of a cancer patient, an HIV patient, or a patient free of cancer and HIV.

9. A method for preparing a factor for treating patients afflicted with a disease that leads to an immunosuppressed state in the patient, which comprises the steps of:
(a) subjecting cytokine-producing lymphocyte cells to mitogenic stimulation in serum-free media for their expansion;
(b) collecting supernatant produced by said mitogenically stimulated cells;
(c) isolating a factor from said supernatant, which comprises a ≧50 kDa fraction thereof.

10. The method of claim 9, wherein said disease results from a persistent or acute virus, a bacterial infection, or an autoimmune disease.

11. The method of claim 10, wherein said persistent or acute virus in an enveloped or non-enveloped RNA or DNA virus.

12. The method of claim 11, wherein said persistent or acute RNA virus is selected from one or more of picornaviruses, togaviruses, paramyxoviruses, orthomyxoviruses, rhandoviruses, reoviruses, retroviruses, bunyaviruses, coronaviruses, and arenaviruses.

13. The method of claim 11, wherein said persistent or acute DNA virus is selected from one or more of panoviruses, papoviruses, adenoviruses, herpesviruses, and poxviruses.

14. The method of claim 10, wherein said disease is one or more of chronic fatigue syndrome (CFS), tuberculosis, measles, dinghy fever, malaria, hepatitis (chronic), leprosy, rheumatoid arthritis, multiple sclerosis, or canine distemper virus.

15. The method of claim 10, wherein said disease comprises chronic fatigue syndrome (CFS).

16. The method of claim 9, wherein said disease is cancer.

17. The method of claim 16, wherein said cancer is an adenocarcinoma.

18. The method of claim 9, wherein said lymphocyte cells are one or more of lymph node lymphocyte cells (LNL) or peripheral blood lymphocyte cells (PBL).

19. The method of claim 9, wherein said mitogenic stimulation includes the presence of Interleukin-2 (IL-2) and anti-CD3 monoclonal antibody.

20. The method of claim 19, wherein said mitogenic stimulation includes the presence of IL2 and soluble anti-CD3 monoclonal antibody followed by re-stimulation in the presence of insoluble anti-CD3 monoclonal antibody.

21. The method of claim 20, wherein said isolate of step (c) is subjected to separation to recover an approximately 70-80 kDa fraction thereof, which is said factor.

22. The method of claim 21, wherein said disease results from a persistent or acute virus, a bacterial infection, a parasite, or an autoimmune disease.

23. The method of claim 22, wherein said persistent or acute virus in an enveloped or non-enveloped RNA or DNA virus.

24. The method of claim 23, wherein said persistent or acute RNA virus is selected from one or more of picornaviruses, togaviruses, paramyxoviruses, orthomyxoviruses, rhandoviruses, reoviruses, retroviruses, bunyaviruses, coronaviruses, and arenaviruses.

25. The method of claim 23, wherein said persistent or acute DNA virus is selected from one or more of panoviruses, papoviruses, adenoviruses, herpesviruses, and poxviruses.

26. The method of claim 22, wherein said disease is one or more of chronic fatigue syndrome (CFS), tuberculosis, measles, dinghy fever, malaria, hepatitis (chronic), leprosy, rheumatoid arthritis, multiple sclerosis, and canine distemper virus.

27. The method of claim 22, wherein said disease comprises chronic fatigue syndrome (CFS).

28. The method of claim 21, wherein said disease is cancer.

29. The method of claim 28, wherein said cancer is an adenocarcinoma.

30. The method of claim 21, wherein said lymphocyte cells are one or more of lymph node lymphocyte cells (LNL) or peripheral blood lymphocyte cells (PBL).

31. The method of claim 21, wherein said mitogenic stimulation includes the presence of Interleukin-2 (IL-2) and anti-CD3 monoclonal antibody.

32. The method of claim 29, wherein said mitogenic stimulation includes the presence of IL2 and soluble anti-CD3 monoclonal antibody followed by re-stimulation in the presence of insoluble anti-CD3 monoclonal antibody.

33. Method for enhancing the activity of one or more of topoisomerase I, topoisomerase II, microtubule, or thymidylate synthetase active agent, which comprises: combining said active agent with a factor, which comprises a ≧50 kDa fraction of a supernatant derived from lymphocyte cells, which have been subjected to mitogenic stimulation in serum free medium.

34. The method of claim 33, wherein said active agent is one ore more of 5-flurouracil, doxorubicin HCI, etoposide phosphate, irinotecan, or gemcitabine HCl.

35. The method of claim 33, wherein said factor is an approximately 70-80 kDa fraction of said ≧50 kDa fraction.

36. Method for enhancing the activity of tamoxifen, which comprises: combining said tamoxifen with a factor, which comprises a ≧50 kDa fraction of a supernatant derived from lymphocyte cells, which have been subjected to mitogenic stimulation in serum free medium.

37. The method of claim 36, wherein said factor is an approximately 70-80 kDa fraction of said ≧50 kDa fraction.

38. A method for treating patients afflicted with human immunodeficiency virus (HIV), which comprises:
administering to said patient an effective dosage of the factor of claim 1.

39. A method for treating patients afflicted with human immunodeficiency virus (HIV), which comprises:
administering to said patient an effective dosage of the factor of claim 2.

40. A method for treating patients afflicted with human immunodeficiency virus (HIV), which comprises:
administering to said patient an effective dosage of the factor of claim 3.

41. A method for treating patients afflicted with human immunodeficiency virus (HIV), which comprises:
administering to said patient an effective dosage of the factor of claim 4.

42. A method for treating patients afflicted with human immunodeficiency virus (HIV), which comprises:
administering to said patient an effective dosage of the factor of claim 5.

43. A method for treating patients afflicted with human immunodeficiency virus (HIV), which comprises:
administering to said patient an effective dosage of the factor of claim 6.

44. A method for treating patients afflicted with human immunodeficiency virus (HIV), which comprises:
administering to said patient an effective dosage of the factor of claim 7.

45. A method for treating patients afflicted with a disease that leads to an immunosuppressed state in the patient, which comprises:
administering to said patient an effective dosage of the factor of claim 1.

46. The method of claim 45, wherein said factor is an approximately 70-80 kDa fraction of said ≧50 kDa fraction.

47. The method of claim 45, wherein said disease results from a persistent or acute virus, a bacterial infection, or an autoimmune disease.

48. The method of claim 47, wherein said persistent or acute virus in an enveloped or non-enveloped RNA or DNA virus.

49. The method of claim 48, wherein said persistent or acute RNA virus is selected from one or more of picornaviruses, togaviruses, paramyxoviruses, orthomyxoviruses, rhandoviruses, reoviruses, retroviruses, bunyaviruses, coronaviruses, and arenaviruses.

50. The method of claim 48, wherein said persistent or acute DNA virus is selected from one or more of panoviruses, papoviruses, adenoviruses, herpesviruses, and poxviruses.

51. The method of claim 45, wherein said disease is one or more of chronic fatigue syndrome (CFS), tuberculosis, measles, dinghy fever, malaria, hepatitis (chronic), leprosy, rheumatoid arthritis, multiple sclerosis, and canine distemper.

52. The method of claim 46, wherein said disease results from a persistent or acute virus, a bacterial infection, or an autoimmune disease.

53. The method of claim 46, wherein said persistent or acute virus in an enveloped or non-enveloped RNA or DNA virus.

54. The method of claim 53, wherein said persistent or acute RNA virus is selected from one or more of picornaviruses, togaviruses, paramyxoviruses, orthomyxoviruses, rhandoviruses, reoviruses, retroviruses, bunyaviruses, coronaviruses, and arenaviruses.

55. The method of claim 53, wherein said persistent or acute DNA virus is selected from one or more of panoviruses, papoviruses, adenoviruses, herpesviruses, and poxviruses.

56. The method of claim 46, wherein said disease is one or more of chronic fatigue syndrome (CFS), tuberculosis, measles, dinghy fever, malaria, hepatitis (chronic), leprosy, rheumatoid arthritis, multiple sclerosis, and canine distemper.

57. A factor for treating a patient afflicted with a disease that leads to an immunosuppressed state in the patient, wherein said factor comprises a protein whose active form has a molecular weight greater than 50 kDa, said protein being derived from lymphocyte cells which have been subjected to mitogenic stimulation.

58. The factor of claim 57, wherein said lymphocyte cells are lymph node lymphocyte cells.

59. The factor of claim 57, wherein said mitogenic stimulation includes the presence of lnterleukin-2 (IL-2) and anti-CD3 monoclonal antibody.

60. The factor of claim 59, wherein said mitogenic stimulation includes the presence of IL2 and soluble anti-CD3 monoclonal antibody followed by re-stimulation in the presence of insoluble anti-CD3 monoclonal antibody.

61. The factor of claim 57, wherein said lymphocyte cells are peripheral blood lymphocyte cells.

62. The factor of claim 57 wherein the active form of said protein has a molecular weight of approximately 70-80 kDa.

63. The factor of claim 60 wherein the active form of said protein has a molecular weight of approximately 70-80 kDa.

64. The factor of claim 57, wherein said lymphocyte cells are derived from one or more of a cancer patient, an HIV patient, or a patient free of cancer and HIV.

65. The factor of claim 57, wherein the active form of said protein is a multimer.

66. The factor of claim 60, wherein the active form of said protein is a multimer.


SEQUENCE LISTING (5 sequences)

SEQ ID NO: 1 - DNA, 21 bp, Homo sapiens
tccttgacacctcagcctcta

SEQ ID NO: 2 - DNA, 21 bp, Homo sapiens
cctcactccagaaagcaggac

SEQ ID NO: 3 - DNA, 20 bp, Homo sapiens
ctttctgctgcgggtaggtg

SEQ ID NO: 4 - DNA, 20 bp, Homo sapiens
gcttgtctcgggtttcrgga

SEQ ID NO: 5 - protein, 12 aa, Homo sapiens
  MOD_RES (1)..(1): X = either S or G
XPAPMMKFFTTX